(19) United States
(12) Patent Application Publication
(10) Pub. No.: US 2022/0205961 A1
(43) Pub. Date: Jun. 30, 2022

(54) METHOD FOR DETERMINING B VITAMINS AND THEIR VITAMERS

(71) Applicant: Waters Technologies Corporation, Milford, MA (US)

(72) Inventors: Jinchuan Yang, Hopkinton, MA (US); Paul Rainville, Princeton, MA (US)

(73) Assignee: Waters Technologies Corporation, Milford, MA (US)

(21) Appl. No.: 17/564,753

(22) Filed: Dec. 29, 2021

Related U.S. Application Data
(60) Provisional application No. 63/132,558, filed on Dec. 31, 2020.

(51) Int. Cl. G01N 30/72; G01N 33/15; G01N 30/60; H01J 49/42

(52) U.S. Cl. CPC G01N 30/7233; G01N 33/15; G01N 2030/027; H01J 49/4215; G01N 30/60

(57) ABSTRACT

The present disclosure discusses a method of separating a sample (e.g., B vitamins and their vitamers) including coating a flow path of a chromatographic system; injecting the sample into the chromatographic system; flowing the sample through the chromatographic system; separating the sample; and analyzing the separated sample. In some examples, the coating applied to the surfaces defining the flow path is non-binding with respect to the sample—and the separated sample. Consequently, the sample does not bind to the low-binding surface of the coating (e.g., organosilica coating) of the flow path. The applied coating provides an inert barrier that is beneficial for B vitamin analysis, including improved peak shape (less tailing and narrower peak), high sensitivity, and no carry-over.

### CROSS-REFERENCE TO RELATED APPLICATIONS

This application claims priority to and the benefit of U.S. Provisional Patent Application No. 63/132,558, filed Dec. 31, 2020, which application is hereby incorporated by reference in its entirety, with any definitions of terms in the present application controlling.

### FIELD OF THE TECHNOLOGY

The present disclosure relates to methods for determining B vitamins and their vitamers. More specifically, this technology relates to analyzing B vitamins and their vitamers via a liquid chromatography (LC) system and column that have been modified with a layer of inert material, for example the LC system and column's internal metal surface has been modified with a deposited inert material.

### BACKGROUND

Liquid chromatography (LC) is an analytical separation technique, which enables the separation of a mixture of chemical species on the basis of differential interactions between the compounds of the mixture and a stationary phase—defined as primary interactions, which are the anticipated interactions between the mixture, the designed stationary phase and the modulations from specifically chosen mobile phase/environmental conditions. These interactions are dependent on a number of controlled variables, such as mobile phase composition, temperature and flow rate.

### SUMMARY

Strong interactions between certain analytes and metal surfaces in liquid chromatography flow path may result in poor chromatographic peak shapes, severe analyte losses, inconsistent peak responses, and other issues in LC, which lead to inaccurate results. To reduce the impact of these interactions, workarounds or additional steps in the methods are often required, which adds complexity to the methods and reduces the laboratory productivity.

Ongoing efforts to reduce chelation and secondary chromatographic interactions of analytes with metal chromatographic surfaces in an effort to facilitate chromatographic separation having higher resolutions are therefore needed. In addition, variability in the separation and detection of compounds can be caused by many factors. One such factor is analyte/surface interactions of compounds with the analytical column. Such interactions can be problematic, especially at very low concentrations of analytes. This is true for B vitamins and their vitamers.

Secondary interaction or adsorption of metal sensitive analytes to active sites dispersed throughout the metallic surface in liquid chromatography based separations have often been challenging to separate. Metal-ion mediated adsorption in liquid chromatography (LC) has been observed as a contributing factor to poor peak shape, tailing, and diminished recovery of sensitive analytes. To address problems experienced in separations in metallic fluidic systems, column hardware using a coating has been developed to define a low-binding surface(s) (LBS).

Using LBS in LC can provide an effective solution to mitigate analyte interactions with metal surfaces. One example is using LBS to analyze B-group vitamins by liquid chromatography, coupled with tandem quadrupole mass spectrometry (LC-MS/MS). B vitamins are essential micronutrients for normal human metabolic and physiological functions. They cannot be synthesized in vivo in sufficient amounts to meet physiological requirements. Abundant supply of vitamins in diet is important for the growth and health of human body. B vitamins are often enriched or fortified in foods. Determination of B vitamins in foods is commonly required in the food manufacturing process. Systems with LBS can enhance LC performance when compared to a conventional system and column. Better peak shapes, increased response, higher sensitivity and no analyte carry-over are some of the potential benefits when using LBS.

For example, using LBS in LC system and columns with B vitamins and their vitamers can help increase the peak area (intensity), reduce peak tailing and peak width for B vitamins (peak shape), which can result in a better response factors and detection sensitivity. The use of LBS in LC systems and columns can also help reduce the potential carry-over issue for B vitamins. And, after extensive use (such as 170 injections), a LBS configuration compared to configuration with no LBS can still exhibit higher response factors (>150%) for some B vitamins, such as thiamine, biotin, nicotinic acid, and 5MTHF.

The benefits of using LBS with B vitamins varies from compound to compound. Some of the B vitamin compounds that experience a benefit associated with LBS systems include flavin mononucleotide, thiamine, thiamine pyrophosphate, pyridoxal 5′-phosphate and pantothenic acid.

No negative impact associated with using LBS has been observed for any B vitamin. Specifically, LBS coated hardware does not appear to adversely affect chromatographic performance or recovery of B vitamins. For example, as discussed herein, after extensive use (such as 170 injections), a LBS configuration compared to configuration with no LBS can still exhibit higher response factors (>150%) for some analytes such as B vitamins and their vitamers.

In addition, sample throughput can be increased by using the technology of the present disclosure. Sample throughput can be increased by reduced peak tailing and increased resolution. For example, if impurities are closely eluting with the native peak and the native peak was exhibiting a degree of tailing, a user (e.g., an analyst) may try to extend the gradient or run-time to resolve impurities to an acceptable resolution between peaks that facilitated accurate quantitation. In the absence of tailing, a user could shorten the run time by using a steeper slope in the gradient. This could effectively elute everything faster and closer together. But the resolution between peaks, while decreasing, may still be sufficient for the analytes of interest since tailing is not present to interfere with integration or cause a co-elution. With reduced peak tailing, new trace species can be detected by being able to see peaks that were formerly covered by peak tailing.

Increased resolution or more time between peaks can allow a user to run faster methods with increased throughput. If resolution has increased, then peak capacity increases meaning more peaks can fit in the same chromatogram or a faster separation could be run at the cost of resolution and peak capacity if the critical pair of interest were resolved sufficiently to start with.

A chromatographic column incorporating the coating of the present disclosure has been designed to minimize negative analyte/surface interactions for compounds, such as compounds with analytes including B vitamins and their vitamers. Existing techniques to mitigate these interactions, such as system passivation with nitric acid, are time consuming and only produce temporary performance gains. It is difficult to determine when the system is fully passivated and ready to operate. If attempts are made to obtain data for quantitative studies before full passivation is reached, the lower end of the calibration curve would not be detected because the analyte still has metallic surfaces it can bind to.

A LBS, such as a surface with an alkylsilyl coating, on the surface area of the flow path of a chromatographic system can minimize the interactions between B vitamins (including their vitamers) and the metallic surfaces of chromatographic flow paths (e.g., column interior walls, interior of tubing, surfaces of frits, sample injectors, etc.). Consequently, the coated metallic surfaces improve liquid chromatography separations for B vitamins and their vitamers. The use of alkylsilyl coatings on metal flow paths allows the use of metal chromatographic flow paths, which are able to withstand high pressures at fast flow rates, high pressure generated using stationary phases with small particles (which can be slow flow as well), and high pressure generated from longer column beds, while minimizing the secondary chromatographic interactions between B vitamin compounds and the metal. These components made of high-pressure material and modified with a coating can be tailored so that the internal flow paths reduce secondary chromatographic interactions. The coating covers the metallic surfaces that are exposed to the fluidic path.

In one aspect, the present disclosure is directed to a method of separating and analyzing a metal-sensitive sample. The method includes injecting the metal-sensitive sample into a chromatographic system, wherein the chromatographic system includes a metallic flow path with a low-bind surface coating; flowing the metal-sensitive sample through the chromatographic system; separating the metal-sensitive sample, wherein the metal-sensitive sample includes one or more B vitamin and/or B vitamin vitamer; and analyzing the separated sample by passing the separated metal-sensitive sample through a mass spectrometer. In some embodiments, the sample includes two or more of B vitamins and/or B vitamin vitamers, and analyzing the separated sample includes simultaneously analyzing the two or more of B vitamins and/or B vitamin vitamers to determine the B vitamins and/or B vitamin vitamers present in the sample. In some embodiments, the method includes, prior to injecting the metal-sensitive sample into the chromatographic system, extracting the metal-sensitive sample from a sample matrix or sample matrices. In some embodiments extracting the metal-sensitive sample from a sample matrix or sample matrices includes separate sample preparations for different B vitamins. In some embodiments, flowing the metal-sensitive sample through the chromatographic system includes using a single liquid chromatography method to determine the B vitamins and/or B vitamin vitamers. In some embodiments, using the single liquid chromatography method includes using liquid chromatography-mass spectrometry (LC/MS). In some embodiments, the liquid chromatography-mass spectrometry (LC/MS) is a tandem quadrupole mass spectrometer (LC-MS/MS). In some embodiments, the B vitamins or B vitamin vitamers include at least one of riboflavin (B₂), flavin mononucleotide (FMN), biotin, nicotinic acid (B₃), nicotinamide (B₃), nicotinamide adenine dinucleotide (NAD), pyridoxine (B₆), pyridoxal (B₆), pyridoxal 5′-phosphate (PLP), thiamine (B₁), thiamine pyrophosphate (TPP), folic acid (B₉), pantothenic acid (B₅), or 5-methyl-tetrahydrofolate (5MTHF). In some embodiments, the sample is a dietary supplement or an energy drink.

In one aspect, the present disclosure is directed a method of separating and analyzing a metal-sensitive sample. The method includes injecting the metal-sensitive sample into a chromatographic system, wherein the chromatographic system includes a metallic flow path with a coating; flowing the metal-sensitive sample through the chromatographic system; separating the metal-sensitive sample, wherein the metal-sensitive sample includes two or more B vitamins and/or B vitamin vitamers; and simultaneously analyzing the two or more B vitamins and/or B vitamin vitamers of the separated sample. In some embodiments, the coating is a low-bind surface coating. In some embodiments, analyzing the separated sample includes passing the separated metal-sensitive sample through a mass spectrometer. In some embodiments, the method includes, prior to injecting the metal-sensitive sample into the chromatographic system, extracting the metal-sensitive sample from a sample matrix or sample matrices. In some embodiments, the method of extracting the metal-sensitive sample from a sample matrix or sample matrices includes separate sample preparations for different B vitamins and/or B vitamin vitamers. In some embodiments, flowing the metal-sensitive sample through the chromatographic system includes using a single liquid chromatography method to determine the B vitamins and/or B vitamin vitamers. In some embodiments, the single liquid chromatography method includes using liquid chromatography-mass spectrometry (LC/MS). In some embodiments, the liquid chromatography-mass spectrometry (LC/MS) is a tandem quadrupole mass spectrometer (LC-MS/MS). In some embodiments, the B vitamins or B vitamin vitamers include riboflavin (B₂), flavin mononucleotide (FMN), biotin, nicotinic acid (B₃), nicotinamide (B₃), nicotinamide adenine dinucleotide (NAD), pyridoxine (B₆), pyridoxal (B₆), pyridoxal 5′-phosphate (PLP), thiamine (B₁), thiamine pyrophosphate (TPP), folic acid (B₉), pantothenic acid (B₅), or 5-methyl-tetrahydrofolate (5MTHF). In some embodiments, the sample is a dietary supplement or an energy drink.

The above aspects and features of the present technology provide numerous advantages over the prior art. In some embodiments, there are numerous benefits incorporating the coating on the column. For example, the present disclosure shows improvement in the LC column and system performance of peak intensity, peak shape, carry-over, and response factor. The present disclosure includes methods for determining B vitamins in energy drinks and dietary supplements.

### BRIEF DESCRIPTION OF THE DRAWINGS

The technology will be more fully understood from the following detailed description taken in conjunction with the accompanying drawings, in which:

FIG. 1 is a schematic of a chromatographic flow system including a chromatography column and various other components, in accordance with an illustrative embodiment of the technology. A fluid is carried through the chromatographic flow system with a fluidic flow path extending from a fluid manager to a detector, such as a MS detector.

FIG. 2 is a flow chart of a method of coating a fluidic path (such as a fluidic path in a chromatography system) according to an illustrative embodiment of the technology.

FIG. 3 is a flow chart showing a method of tailoring a fluidic flow path for separation of a sample including a B vitamin and/or B vitamin vitamer, in accordance with an illustrative embodiment of the technology.

FIG. 4A, FIG. 4B, FIG. 4C, FIG. 4D, FIG. 4E, FIG. 4F, FIF. 4G, FIG. 4H, FIG. 4I, FIG. 4J, FIG. 4K, FIG. 4L, FIG. 4M, FIG. 4N, and FIG. 4O display structures of selected B vitamins. FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, FIG. 4N, and FIG. 4O display structures of vitamin B₁₂, riboflavin (B₂), biotin, FMN (flavin mononucleotide), nicotinic acid (B₃), nicotinamide (B₃), nicotinamide adenine dinucleotide, pyridoxine (B₆), pyridoxamine (B₆), pyridoxal (B₆), PLP (Pyridoxal 5′-phosphate), thiamine (B₁), folic acid (B₉), 5MTHF (5-methyl-tetrahydrofolate), TPP (Thiamine pyrophosphate), and pantothenic acid (B₅), respectively.

FIG. 5 displays the liquid chromatography conditions in the Association of Official Analytical Chemists (AOAC) Official Method for simultaneous determination of total vitamins B₁, B₂, B₃, and B₆ and other nutrients in matrices, such as infant formula and related nutritionals.

FIG. 6A and FIG. 6B display the multiple reaction monitoring (MRM) transitions and MS detection parameters for 18 vitamins, including the additional vitamins B₅, B₉, B₁₂ and biotin, that were used for the Xevo™ TQ-S Micro MS System.

FIG. 7 displays a table of calibration data, in accordance with the present disclosure.

FIG. 8A, FIG. 8B, FIG. 8C, FIG. 8D, FIG. 8E, FIG. 8F, FIG. 8G, FIG. 8H, FIG. 8I, FIG. 8J, FIG. 8K, FIG. 8L, FIG. 8M, FIG. 8N, FIG. 8O, FIG. 8P, FIG. 8Q and FIG. 8R show comparison plots of the vitamin peak areas from 7 replicate injections of a standard mix solution (at a concentration of 1 μg/mL) in the SOP configuration and the LBS configuration. FIG. 8A, FIG. 8B, FIG. 8C, FIG. 8D, FIG. 8E, FIG. 8F, FIG. 8G, FIG. 8H, FIG. 8I, FIG. 8J, FIG. 8K, FIG. 8L, FIG. 8M, FIG. 8N, FIG. 8O, FIG. 8P, FIG. 8Q and FIG. 8R display peak areas for pyridoxine, thiamine, MeB12 (methyl-cobalamine), TPP (Thiamine pyrophosphate), Aden B12 (adenosyl-cobalamin), nicotinic acid, riboflavin, nicotinamide, FMN (flavin mononucleotide), nicotinamide adenine dinucleotide (NAD), B₁₂, folic acid, pyridoxamine, 5MTHF (5-methyl-tetrahydrofolate), pyridoxal, pantothenic acid, PLP (Pyridoxal 5′-phosphate), and biotin, respectively, in accordance with the present disclosure.

FIG. 9A, FIG. 9B, FIG. 9C, FIG. 9D, FIG. 9E, FIG. 9F, FIG. 9G, and FIG. 9H compare the chromatograms for the LBS configuration and the SOP configuration for flavin mononucleotide (FMN), thiamine (B₁), pyridoxal 5′-phosphate (PLP), and pantothenic acid (B₅), respectively, in accordance with the present disclosure. FIG. 9A, FIG. 9C, FIG. 9E, and FIG. 9G display the chromatograms for the LBS configuration for flavin mononucleotide (FMN), thiamine (B₁), pyridoxal 5′-phosphate (PLP), and pantothenic acid (B₅), respectively, in accordance with the present disclosure. FIG. 9B, FIG. 9D, FIG. 9F, and FIG. 9H display the chromatograms for the SOP configuration for flavin mononucleotide (FMN), thiamine (B₁), pyridoxal 5′-phosphate (PLP), and pantothenic acid (B₅), respectively, in accordance with the present disclosure.

FIG. 10A, FIG. 10B, FIG. 10C, and FIG. 10D are comparisons of LC-MS chromatograms of FMN (FIG. 10A), Thiamine (FIG. 10B), PLP (FIG. 10C), and Pantothenic acid (FIG. 10D) from the initial injection of in the same standard solution using LBS surfaces (LBS configuration) vs standard surfaces (SOP configuration), in accordance with the present disclosure.

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L, FIG. 11M, FIG. 11N compare thiamine pyrophosphate (TPP) chromatograms from initial seven injections for the SOP configuration and the LBS configuration, in accordance with the present disclosure. FIG. 11A, FIG. 11B, FIG. 11C, FIG. 11D, FIG. 11E, FIG. 11F, FIG. 11G display chromatograms from 7^{th}, 6^{th}, 5^{th}, 4^{th}, 3^{rd}, 2^{nd} and 1^{st} injections for the SOP configuration, respectively. FIG. 11H, FIG. 11I, FIG. 11J, FIG. 11K, FIG. 11L, FIG. 11M, FIG. 11N display chromatograms from 7^{th}, 6^{th}, 5^{th}, 4^{th}, 3^{rd}, 2^{nd} and 1^{st} injections for the LBS configuration.

FIG. 12A, FIG. 12B, FIG. 12C, FIG. 12D, FIG. 12E, FIG. 12F, FIG. 12G, FIG. 12H, FIG. 12I, FIG. 12J, FIG. 12K, FIG. 12L, FIG. 12M, FIG. 12N, FIG. 12O and FIG. 12P compare the chromatograms of flavin mononucleotide (FMN) in standard solutions for the SOP configuration and the LBS configuration, in accordance with the present disclosure. FIG. 12A, FIG. 12B, FIG. 12C, FIG. 12D, FIG. 12E, FIG. 12F, FIG. 12G, FIG. 12H display chromatograms for 10 ppm, 3 ppm, 1000 ppb, 300 ppb, 100 ppb, 30 ppb, 10 ppb, and 3 ppb of flavin mononucleotide (FMN) concentrations, respectively, in standard solutions for the SOP configuration. FIG. 12I, FIG. 12J, FIG. 12K, FIG. 12L, FIG. 12M, FIG. 12N, FIG. 12O and FIG. 12P display chromatograms for 10 ppm, 3 ppm, 1000 ppb, 300 ppb, 100 ppb, 30 ppb, 10 ppb, and 3 ppb of flavin mononucleotide (FMN) concentrations, respectively, in standard solutions for the LBS configuration.

FIG. 13A, FIG. 13B, FIG. 13C, FIG. 13D, FIG. 13E, FIG. 13F, FIG. 13G, FIG. 13H, FIG. 13I, FIG. 13J, FIG. 13K, FIG. 13L, FIG. 13M, FIG. 13N, FIG. 13O and FIG. 13P compare the chromatograms of pyridoxal 5′-phosphate (PLP) in standard solutions for the SOP configuration and the LBS configuration, in accordance with the present disclosure. FIG. 13A, FIG. 13B, FIG. 13C, FIG. 13D, FIG. 13E, FIG. 13F, FIG. 13G and FIG. 13H display chromatograms for 10 ppm, 3 ppm, 1000 ppb, 300 ppb, 100 ppb, 30 ppb, 10 ppb, and 3 ppb of pyridoxal 5′-phosphate (PLP) concentrations, respectively, in standard solutions for the SOP configuration. FIG. 13I, FIG. 13J, FIG. 13K, FIG. 13L, FIG. 13M, FIG. 13N, FIG. 13O and FIG. 13P display chromatograms for 10 ppm, 3 ppm, 1000 ppb, 300 ppb, 100 ppb, 30 ppb, 10 ppb, and 3 ppb of pyridoxal 5′-phosphate (PLP) concentrations, respectively, in standard solutions for the LBS configuration.

FIG. 14A, FIG. 14B, FIG. 14C, FIG. 14D, FIG. 14E, FIG. 14F, FIG. 14G, FIG. 14H, FIG. 14I, FIG. 14J, FIG. 14K, FIG. 14L, FIG. 14M, FIG. 14N, FIG. 14O and FIG. 14P compare the chromatograms of thiamine in standard solutions for the SOP configuration and the LBS configuration, in accordance with the present disclosure. FIG. 14A, FIG. 14B, FIG. 14C, FIG. 14D, FIG. 14E, FIG. 14F, FIG. 14G and FIG. 14H display chromatograms for 10 ppm, 3 ppm, 1000 ppb, 300 ppb, 100 ppb, 30 ppb, 10 ppb, and 3 ppb of thiamine concentrations, respectively, in standard solutions for the SOP configuration. FIG. 14I, FIG. 14J, FIG. 14K, FIG. 14L, FIG. 14M, FIG. 14N, FIG. 14O and FIG. 14P display chromatograms for 10 ppm, 3 ppm, 1000 ppb, 300 ppb, 100 ppb, 30 ppb, 10 ppb, and 3 ppb of thiamine concentrations, respectively, in standard solutions for the LBS configuration.

FIG. 15A, FIG. 15B, FIG. 15C, FIG. 15D, FIG. 15E, FIG. 15F, FIG. 15G, FIG. 15H, FIG. 15I, FIG. 15J, FIG. 15K, FIG. 15L, FIG. 15M, FIG. 15N, FIG. 15O and FIG. 15P compare the chromatograms of pantothenic acid in standard solutions for the SOP configuration and the LBS configuration, in accordance with the present disclosure. FIG. 15A, FIG. 15B, FIG. 15C, FIG. 15D, FIG. 15E, FIG. 15F, FIG. 15G and FIG. 15H display chromatograms for 10 ppm, 3 ppm, 1000 ppb, 300 ppb, 100 ppb, 30 ppb, 10 ppb, and 3 ppb of pantothenic acid concentrations, respectively, in standard solutions for the SOP configuration. FIG. 15I, FIG. 15J, FIG. 15K, FIG. 15L, FIG. 15M, FIG. 15N, FIG. 15O and FIG. 15P display chromatograms for 10 ppm, 3 ppm, 1000 ppb, 300 ppb, 100 ppb, 30 ppb, 10 ppb, and 3 ppb of pantothenic acid concentrations, respectively, in standard solutions for the LBS configuration.

FIG. 16A, FIG. 16B, FIG. 16C, FIG. 16D, FIG. 16E, FIG. 16F, FIG. 16G, FIG. 16H, FIG. 16I, FIG. 16J, FIG. 16K, FIG. 16L, FIG. 16M, FIG. 16N, FIG. 16O and FIG. 16P compare the chromatograms of nicotinic acid in standard solutions for the SOP configuration and the LBS configuration, in accordance with the present disclosure. FIG. 16A, FIG. 16B, FIG. 16C, FIG. 16D, FIG. 16E, FIG. 16F, FIG. 16G and FIG. 16H display chromatograms for 10 ppm, 3 ppm, 1000 ppb, 300 ppb, 100 ppb, 30 ppb, 10 ppb, and 3 ppb of nicotinic acid concentrations, respectively, in standard solutions for the SOP configuration. FIG. 16I, FIG. 16J, FIG. 16K, FIG. 16L, FIG. 16M, FIG. 16N, FIG. 16O and FIG. 16P display chromatograms for 10 ppm, 3 ppm, 1000 ppb, 300 ppb, 100 ppb, 30 ppb, 10 ppb, and 3 ppb of nicotinic acid concentrations, respectively, in standard solutions for the LBS configuration.

FIG. 17A, FIG. 17B, FIG. 17C, FIG. 17D, FIG. 17E, FIG. 17F, FIG. 17G, FIG. 17H, FIG. 17I, FIG. 17J, FIG. 17K, FIG. 17L, FIG. 17M, FIG. 17N, FIG. 17O, FIG. 17P and FIG. 17Q compare peak areas for the SOP configuration and LBS configuration after initial injection when the systems and columns had been used lightly. FIG. 17A, FIG. 17B, FIG. 17C, FIG. 17D, FIG. 17E, FIG. 17F, FIG. 17G, FIG. 17H, FIG. 17I, FIG. 17J, FIG. 17K, FIG. 17L, FIG. 17M, FIG. 17N, FIG. 17O, FIG. 17P and FIG. 17Q display result for selected B vitamins, namely; B12, thiamine, MeB12, TPP, Aden B12, nicotinic acid, pyridoxine, folic acid, pyridoxamine, 5MTHF, pyridoxal, pantothenic acid, riboflavin, PLP, nicotinamide, biotin and FMN, respectively, in accordance with the present disclosure.

FIG. 18A, FIG. 18B, FIG. 18C, FIG. 18D, FIG. 18E, FIG. 18F, FIG. 18G, FIG. 18H, FIG. 18I and FIG. 18J compare the chromatograms for the LBS configuration and the SOP configuration for selected B vitamins. FIG. 18A, FIG. 18C, FIG. 18E, FIG. 18G and FIG. 18I display the chromatograms for the LBS configuration for flavin mononucleotide (FMN), thiamine (B₁), pyridoxal 5′-phosphate (PLP), pantothenic acid (B₅), and thiamine pyrophosphate (TPP) respectively, in accordance with the present disclosure. FIG. 18B, FIG. 18D, FIG. 18F, FIG. 18H and FIG. 18J display the chromatograms for the SOP configuration for flavin mononucleotide (FMN), thiamine (B₁), pyridoxal 5′-phosphate (PLP), pantothenic acid (B₅), and thiamine pyrophosphate (TPP) respectively, in accordance with the present disclosure.

FIG. 19A, FIG. 19B, FIG. 19C, FIG. 19D, FIG. 19E, FIG. 19F, FIG. 19G, FIG. 19H, FIG. 19I, FIG. 19J, FIG. 19K and FIG. 19L show a comparison of the selected B vitamins LC-MS chromatograms obtained with the LBS and the SOP configurations in a carry-over study. FIG. 19A, FIG. 19D, FIG. 19G and FIG. 19J display LC-MS chromatograms of injections of a 10 ppm standard solution for riboflavin, pyridoxal, 5-methyl-THF, and methylcobalamin (Me B12), respectively, in accordance with the present disclosure. FIG. 19B, FIG. 19E, FIG. 19H and FIG. 19K display LC-MS chromatograms of blank injections obtained with the SOP configurations for riboflavin, pyridoxal, 5-methyl-THF, and methylcobalamin (Me B12), respectively, in accordance with the present disclosure. FIG. 19C, FIG. 19F, FIG. 19I and FIG. 19L display LC-MS chromatograms of blank injections obtained with the LBS configurations for riboflavin, pyridoxal, 5-methyl-THF, and methylcobalamin (Me B12), respectively, in accordance with the present disclosure.

FIG. 20A, FIG. 20B, FIG. 20C and FIG. 20D display the chromatograms for the LBS configuration and the SOP configuration for selected B vitamins to compare carry-over for riboflavin. FIG. 20B and FIG. 20A show a blank injection (FIG. 20A) right after an injection of a high concentration standard solution (10 ppm) (FIG. 20B) for LBS configuration. FIG. 20C and FIG. 20D show a blank injection (FIG. 20D) right after an injection of a high concentration standard solution (10 ppm) (FIG. 20C) for SOP configuration.

FIG. 21A, FIG. 21B, FIG. 21C and FIG. 21D display the chromatograms for the LBS configuration and the SOP configuration for selected B vitamins to compare carry-over for pyridoxal. FIG. 21B and FIG. 21A show a blank injection (FIG. 21B) right after an injection of a high concentration standard solution (10 ppm) (FIG. 21A) for LBS configuration. FIG. 21C and FIG. 21D show a blank injection (FIG. 21D) right after an injection of a high concentration standard solution (10 ppm) (FIG. 21C) for SOP configuration.

FIG. 22A, FIG. 22B, FIG. 22C and FIG. 22D display the chromatograms for the LBS configuration and the SOP configuration for selected B vitamins to compare carry-over for 5-methyl-THF. FIG. 22B and FIG. 22A show a blank injection right after an injection of a high concentration standard solution (10 ppm) for LBS configuration. FIG. 22C and FIG. 22D show a blank injection right after an injection of a high concentration standard solution (10 ppm) for SOP configuration.

FIG. 23A, FIG. 23B, FIG. 23C and FIG. 23D display the chromatograms for the LBS configuration and the SOP configuration for selected B vitamins to compare carry-over for methyl-cobalamin. FIG. 23B and FIG. 23A show a blank injection (FIG. 23B) right after an injection of a high concentration standard solution (10 ppm) (FIG. 20A) for LBS configuration. FIG. 23C and FIG. 23D show a blank injection (FIG. 23D) right after an injection of a high concentration standard solution (10 ppm) (FIG. 23) for SOP configuration.

FIG. 24 displays the relative response factors for B vitamins on the LC-MS systems comparing the LBS configuration to the SOP configuration during the LC-MS analysis of B vitamins.

FIG. 25A, FIG. 25B, FIG. 25C, FIG. 25D, FIG. 25E, FIG. 25F, FIG. 25G, FIG. 25H, FIG. 25I, FIG. 25J, FIG. 25K, FIG. 25L, FIG. 25M, FIG. 25N, FIG. 25O and FIG. 25P displays typical chromatograms of selected B vitamins, namely; thiamine, PLP (Pyridoxal 5′-phosphate), pyridoxine, pyridoxal, pyridoxamine, nicotinic acid, pantothenic acid, nicotinamide, CN B12 (cyanocobalamine), MeB12 (methyl-cobalamine), 5MHTF (5-methyl-tetrahydrofolate), FA (folic acid), AdenB12 (adenosyl-cobalamin), FMN (flavin mononucleotide), riboflavin, and biotin, respectively, in accordance with the present disclosure.

FIG. 26A, FIG. 26B, FIG. 26C, FIG. 26D, FIG. 26E, FIG. 26F, FIG. 26G and FIG. 26H displays a typical chromatogram of selected B vitamins in an energy drink sample. FIG. 26I, FIG. 26J, FIG. 26K, FIG. 26L, FIG. 26M, FIG. 26N, FIG. 26O, FIG. 26P, FIG. 26Q, FIG. 26R and FIG. 26S displays a typical chromatogram of selected B vitamins in a dietary supplement sample. FIG. 26A, FIG. 26B, FIG. 26C, FIG. 26D, FIG. 26E, FIG. 26F, FIG. 26G and FIG. 26H displays a typical chromatogram of CN B12, FMN, riboflavin, pyridoxal, nicotinic acid, pantothenic acid, nicotinamide, respectively, in accordance with the present disclosure. FIG. 26I, FIG. 26J, FIG. 26K, FIG. 26L, FIG. 26M, FIG. 26N, FIG. 26O, FIG. 26P, FIG. 26Q, FIG. 26R and FIG. 26S displays a typical chromatogram of CN B12, FA (folic acid), FMN, riboflavin, biotin, thiamine, pyridoxine, pyridoxal, nicotinic acid, pantothenic acid, and nicotinamide, respectively, in accordance with the present disclosure.

FIG. 27 displays the table of energy drink sample analysis with recovery and repeatability.

FIG. 28 displays the table of dietary supplement sample analysis with recovery and repeatability.

### DETAILED DESCRIPTION

In general, the present disclosure is related to coating columns to have LBS to increase peak intensity, peak shape, carry-over, and response factor by minimizing negative analyte/surface interactions that can lead to sample losses. The present disclosure addresses the problematic binding of B vitamins and their vitamers on metallic surfaces of chromatographic systems. For example, B vitamins can interact with stainless steel to reduce peak intensity.

In addition, coating the system to have a LBS minimizes uncertainty of the chromatographic system performance. Permanent passivation (or at least semi-permanent passivation, i.e., useable lifetime of a consumable) can be provided by the coating. For example, the system does not need to be passivated after each wash, and passivation does not effectively diminish after each wash or flowing. Consequently, the analyte detected using LC and a detector (e.g., MS, UV (for abundant species), etc.) can be depended upon as an accurate assessment of the analyte present.

Analytes that interact with metal have often proven to be very challenging to separate. The desire to have high pressure capable chromatographic systems with minimal dispersion has required that flow paths decrease in diameter and be able to withstand increasingly high pressures at increasingly fast flow rates. As a result, the material of choice for chromatographic flow paths is often metallic in nature. This is despite the fact that characteristics of certain analytes, for example, biomolecules, proteins, glycans, peptides, oligonucleotides, pesticides, bisphosphonic acids, anionic metabolites, and zwitterions like amino acids and neurotransmitters, are known to have unfavorable interactions, so called chromatographic secondary interactions, with metallic surfaces.

The proposed mechanism for metal specific binding interactions requires an understanding of the Lewis theory of acid-base chemistry. Pure metals and metal alloys (along with their corresponding oxide layers) have terminal metal atoms that have characteristics of a Lewis acid. More simply, these metal atoms show a propensity to accept donor electrons. This propensity is even more pronounced with any surface metal ions bearing a positive charge. Analytes with sufficient Lewis base characteristics (any substance that can donate non-bonding electrons) can potentially adsorb to these sites and thus form problematic non-covalent complexes. It is these substances that are defined as metal-interacting analytes.

For example, B vitamins and their vitamers are capable of metal chelation. This interaction causes the B vitamins and their vitamers to bind to the flow path metals thus reducing the detected amounts of such species, a particularly troublesome effect when B vitamins are the most important analyte and have low concentration levels in the sample.

Other characteristics of analytes can likewise pose problems. For example, when the functional groups are ubiquitous in the sample, giving the opportunity for cumulative analyte losses or undesirable chromatographic performance.

The inner surface of the flow path in LC systems and columns has a relatively small surface area as compared to the surface area of the packing material in the chromatography column. Despite the small surface area, the inner surface in the flow path should not be overlooked as a potential source of unwanted interactions with target analytes. Strong interactions between the metallic surfaces and analytes can lead to surface adsorption of analytes which may result in poor peak shape, reduced or no peak response, and inaccurate results. There are workarounds to address these issues, such as replacing the stainless-steel material with other materials, using additives in the mobile phases to disturb the interactions, or coating the surface with strong adsorbents prior to the analysis. These workarounds have their limitations and may negatively impact the chromatography performance and analytical productivity.

For example, an alternative to using metal flow paths is to use flow paths constructed from polymeric materials, such as polyether ether ketone (PEEK). PEEK tubing, like most polymeric materials, is formed by means of an extrusion process. With polymeric resin, this manufacturing process can lead to highly variable internal diameters. Accordingly, PEEK column hardware yields unfavorable differences in the retention times as can be observed from switching between one column and the next. Often, this variation can be a factor of three higher than a metal constructed column. In addition, the techniques for fabricating polymer based frits are not yet sufficiently developed to afford suitably rugged components for commercial HPLC columns. For example, commercially available PEEK frits tend to exhibit unacceptably low permeability.

One method of coating for LBS is the use of alkylsilyl coatings. In some aspects, the alkylsilyl coating acts a bioinert, low-bind coating to modify a flow path to address flow path interactions with an analyte, such as a metal-sensitive analyte. That is, the bioinert, low-bind coating minimizes surface reactions with the metal interacting compounds and allows the sample to pass along a flow path without clogging, attaching to surfaces, or change in analyte properties. The reduction/elimination of these interactions is advantageous because it allows for accurate quantification and analysis of a sample containing B vitamin compounds or other metal-sensitive compounds. The coating which creates LBS along the flow path prevents/significantly minimizes analyte loss to the metallic surface walls, thereby reducing secondary chromatographic interactions.

LBS due to the coatings can provide an effective solution that mitigates analyte interactions with metal surfaces. Coatings used to create a LBS can include a highly cross-linked layer containing ethylene-bridged siloxane material that is similar to that of BEH particles. Relevant analytes include organic acids, organophosphates, oligonucleotides, peptide, glycans, and phospholipids. B vitamins are water-soluble vitamins that are essential for normal human metabolic and physiological functions. The routine testing of B vitamins is an important procedure for food, beverage and nutraceutical quality control as well as for nutritional research. The present disclosure discussed LBS in the LC-MS/MS analysis of B vitamins.

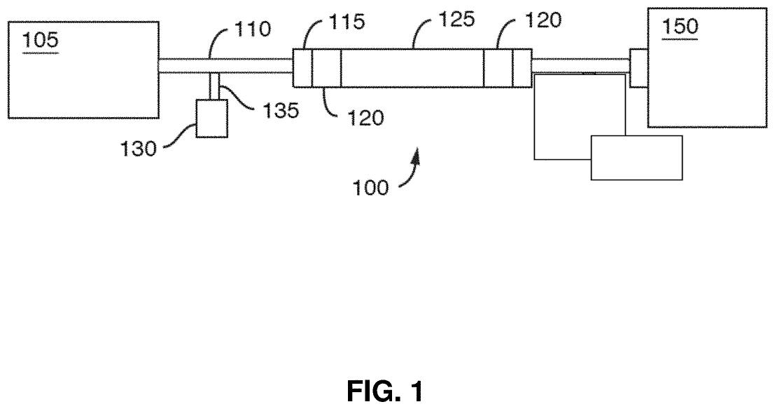

FIG. 1 is a representative schematic of a chromatographic system/device 100 that can be used to separate analytes, such as B vitamins and their vitamers, in a sample. System 100 includes several components including a fluid manager system 105 (e.g., controls mobile phase flow through the system), tubing 110 (which could also be replaced or used together with micro fabricated fluid conduits), fluid connectors 115, frits 120, a chromatography column 125, a sample injector 135 including a needle (not shown) to insert or inject the sample into the mobile phase, a vial, or sample container 130 for holding the sample prior to injection, and a detector 150, such as a mass spectrometer. Chromatography column 125 can be a reversed phase column. Interior surfaces of the components of the chromatographic system/device 100 form a fluidic flow path that has wetted surfaces. Components of the fluidic flow path can have a length to diameter ratio of at least 20, at least 25, at least 30, at least 35 or at least 40. The fluidic flow path can include wetted surfaces of an electrospray needle (not shown).

At least a portion of the wetted surfaces can be LBS by coating with an alkylsilyl coating to reduce secondary interactions. The coating can tailor the hydrophobicity of the wetted surfaces. The coating can be applied by vapor deposition. As such, methods and devices of the present technology provide the advantage of being able to use high pressure resistant materials (e.g., stainless steel) of a flow system, and the wetted surfaces of the fluidic flow path providing the appropriate hydrophobicity so deleterious interactions or undesirable chemical effects on the sample can be minimized.

In some examples, the coating of the flow path is non-binding with respect to the analyte, such as a metal-sensitive compound (e.g., B vitamins). Consequently, the analyte does not bind to the coating of the flow path.

The coating can be provided throughout the system from the tubing or fluid conduits 110 extending from the fluid manager system 105 all the way through to the detector 150. The coatings can also be applied to components of the fluidic path. That is, one may choose to coat one or more components or portions of a component and not the entire fluidic path. For example, the internal portions of the column 125 and its frits 120 and fluid connectors 115 can be coated whereas the remainder of the flow path can be left unmodified. Further, removable/replaceable components can be coated. For example, the vial 130 containing the sample can be coated as well as frits 120.

In some examples, system 100 will need to be cleaned/cleared before evaluation begins in order to establish a baseline before beginning tests to determine suitability. Ensuring system 100 is at a baseline can help certify that there are no contaminants. It can also be used to validate a preparation process for system 100 after manufacturing of system 100 is complete. For example, after system 100 is manufactured, method 300 of FIG. 3 (discussed below) can be used.

The flow path of the fluidic systems can be defined at least in part by an interior surface of tubing. The flow path of the fluidic systems can also be described at least in part by an interior surface of microfabricated fluid conduits. And the flow path of the fluidic systems can be described as at least in part by an interior surface of a column or at least in part by passageways through a frit. The flow path of the fluidic systems is also described at least in part by an interior surface of a sample injection needle or extending from the interior surface of a sample injection needle throughout the interior surface of a column. In addition, the flow path can be described as extending from a sample container (e.g., a vial) disposed upstream of and in fluidic communication with the interior surface of a sample injection needle throughout the fluidic system to a connector/port to a detector. In some examples, all tubing, connectors, frits, membranes, sample reservoirs, and fluidic passageways along this fluidic path (wetted surfaces) are coated.

In some examples, only the wetted surfaces of the chromatographic column and the components located upstream of the chromatographic column are LBS, e.g., coated with an alkylsilyl coating, while wetted surfaces located downstream of the column are not coated. In other embodiments, all wetted surfaces are coated, including those surfaces downstream of the column. And in certain embodiments, wetted surfaces upstream of the column, through the column, and downstream of the column to the entrance of inlet to the detector are coated. The coating can be applied to the wetted surfaces via vapor deposition. Similarly, the “wetted surfaces” of labware or other fluid processing devices may benefit from alkylsilyl coatings. The “wetted surfaces” of these devices not only include the fluidic flow path, but also elements that reside within the fluidic flow path. For example, frits and/or membranes within a solid phase extraction device come in contact with fluidic samples. As a result, not only the internal walls within a solid phase extraction device, but also any frits/membranes are included within the scope of “wetted surfaces.” All “wetted surfaces” or at least some portion of the “wetted surfaces” can be improved or tailored for a particular analysis or procedure by including one or more of the coatings described herein. The term “wetted surfaces” refers to all surfaces within a device (e.g., chromatography column, chromatography injection system, chromatography fluid handling system, frits, labware, solid phase extraction device, pipette tips, centrifuge tubes, beakers, dialysis chambers) that come into contact with a fluid, especially a fluid containing an analyte of interest.

Further information regarding the coating and the deposition of coatings in accordance with the present technology is available in US 2019/0086371, which is hereby incorporated by reference.

A LBS configuration can include an alkylsilyl coating, as discussed herein. For example, a LBS configuration can include a C₂ coating, as discussed herein.

At least a portion of the wetted surfaces are coated with an alkylsilyl coating. The alkylsilyl coating is inert to at least one of the analytes in the sample. The alkylsilyl coating can have the Formula I:

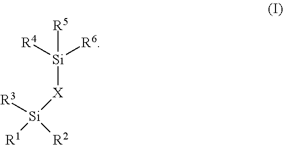

R¹, R², R³, R⁴, R⁵, and R⁶ are each independently selected from (C₁-C₆)alkoxy, —NH(C₁-C₆)alkyl, —N((C₁-C₆)alkyl)₂, OH, OR^{A}, and halo (i.e., a halogen, for example chloro). R^{A} represents a point of attachment to the interior surfaces of the fluidic system. At least one of R¹, R², R³, R⁴, R⁵, and R⁶ is OR^{A}. X is (C₁-C₂₀)alkyl, —O[(CH₂)₂O]₁₋₂₀—, —(C₁-C₁₀)[NH(CO)NH(C₁-C₁₀)]₁₋₂₀-, or —(C₁-C₁₀)[alkylphenyl(C₁-C₁₀)alkyl]₁₋₂₀-.

When used in the context of a chemical formula, a hyphen (“-”) indicates the point of attachment. For example, when X is —[(C₁-C₁₀)alkylphenyl(C₁-C₁₀)alkyl]₁₋₂₀-, that means that X is connected to SiR¹R²R³ via the (C₁-C₁₀)alkyl and connected to SiR⁴R⁵R⁶ via the other (C₁-C₁₀)alkyl. This applies to the remaining variables.

In one aspect, X in Formula I is (C₁-C₁₅)alkyl, (C₁-C₁₂)alkyl, or (C₁-C₁₀)alkyl. In some aspects, X in Formula I is methyl, ethyl, propyl, isopropyl, butyl, sec-butyl, iso-butyl, t-butyl, pentyl, hexyl, heptyl, nonyl, or decanyl. In other aspect, X in Formula I is ethyl or decanyl.

In one aspect, at least one of R¹, R², R³, R⁴, R⁵, and R⁶ is (C₁-C₆)alkoxy, e.g., ethoxy, wherein the values for X are described in Formula I or the preceding paragraph. In another aspect, at least two of R¹, R², R³, R⁴, R⁵, and R⁶ is (C₁-C₆)alkoxy, e.g., ethoxy, wherein the values for X are described in Formula I or the preceding paragraph. In another aspect, at least three of R¹, R², R³, R⁴, R⁵, and R⁶ is (C₁-C₆)alkoxy, e.g., ethoxy, wherein the values for X are described in Formula I or the preceding paragraph. In another aspect, at least four of R¹, R², R³, R⁴, R⁵, and R⁶ is (C₁-C₆)alkoxy, e.g., ethoxy, wherein the values for X are described in Formula I or the preceding paragraph. In another aspect, at least five of R¹, R², R³, R⁴, R⁵, and R⁶ is (C₁-C₆)alkoxy, e.g., ethoxy, wherein the values for X are described in Formula I or the preceding paragraph.

In one aspect, at least one of R¹, R², R³, R⁴, R⁵, and R⁶ is halo, e.g., chloro, wherein the values for X are described in Formula I or the preceding paragraphs above. In another aspect, at least two of R¹, R², R³, R⁴, R⁵, and R⁶ is halo, e.g., chloro, wherein the values for X are described in Formula I or the preceding paragraphs above. In another aspect, at least three of R¹, R², R³, R⁴, R⁵, and R⁶ is halo, e.g., chloro, wherein the values for X are described in Formula I or the preceding paragraphs above. In another aspect, at least four of R¹, R², R³, R⁴, R₅, and R⁶ is halo, e.g., chloro, wherein the values for X are described in Formula I or the preceding paragraphs above. In another aspect, at least five of R¹, R², R³, R⁴, R₅, and R⁶ is halo, e.g., chloro, wherein the values for X are described in Formula I or the preceding paragraphs above.

In another aspect, R¹, R², R³, R⁴, R₅, and R⁶ are each methoxy or chloro.

In some embodiments, the alkylsilyl coating of Formula I is a organosilica coating. In certain embodiments, the alkylsilyl coating of Formula I is a hybrid inorganic/organic material that forms the wetted surface or that coats the wetted surfaces.

The alkylsilyl coating of Formula I can have a contact angle of at least about 15°. In some embodiments, the alkylsilyl coating of Formula I can have a contact angle of less than or equal to 30°. The contact angle can be less than or equal to about 115°. In some embodiments, the contact angle of the alkylsilyl coating of Formula I is between about 150 to about 90°, in some embodiments about 150 to about 105°, and in some embodiments about 150 to about 115°. For example, the contact angle of the alkylsilyl coating of Formula I can be about 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, or 115°.

The thickness of the alkylsilyl coating can be at least about 100 Å. For example, the thickness can be between about 100 Å to about 1600 Å. The thickness of the alkylsilyl coating for Formal I can be about 100 Å, 200 Å, 300 Å, 400 Å, 500 Å, 600 Å, 700 Å, 800 Å, 900 Å, 1000 Å, 1100 Å, 1200 Å, 1300 Å, 1400 Å, 1500 Å or 1600 Å. The thickness of the alkylsilyl coating (e.g., a vapor deposited alkylsilyl coating) can be detected optically by the naked eye. For example, more opaqueness and coloration is indicative of a thicker coating. Thus, coatings with pronounced visual distinction are an embodiment of this technology. From thin to thick, the color changes from yellow, to violet, to blue, to slightly greenish and then back to yellow when coated parts are observed under full-spectrum light, such as sunlight. For example, when the alkylsilyl coating is 300 Å thick, the coating can appear yellow and reflect light with a peak wavelength between 560 and 590 nm. When the alkylsilyl coating is 600 Å thick, the coating can appear violet and reflect light with a peak wavelength between 400 and 450 nm. When the alkylsilyl coating is 1000 Å thick, the coating can appear blue and reflect light with a peak wavelength between 450 and 490 nm. See, e.g., Faucheu et al., Relating Gloss Loss to Topographical Features of a PVDF Coating, Published Oct. 6, 2004; Bohlin, Erik, Surface and Porous Structure of Pigment Coatings, Interactions with flexographic ink and effects of print quality, Dissertation, Karlstad University Studies, 2013:49.

In one aspect, the vapor deposited coating of Formula I is the product of vapor deposited bis(trichlorosilyl)ethane, bis(trimethoxysilyl)ethane, bis(trichlorosilyl)octane, bis(trimethoxysilyl)octane, bis(trimethoxysilyl)hexane, and bis(trichlorosilyl)hexane.

In some aspects, at least a portion of the wetted surfaces are coated with multiple layers of the same or different alkyslilyls, where the thickness of the alkylsilyl coatings correlate with the number of layering steps performed (e.g., the number of deposited layers of alkylsilyl coating on wetted surfaces (e.g., internal surfaces of the fluidic flow path of the chromatographic system/device or internal surfaces or fluid interfacing/contacting surfaces of labware or other analytical devices, such as frits within a solid phase extraction device together with interior walls of the solid phase extraction device). In this manner, increasingly thick bioinert coatings can be produced and tailored to achieve desirable separations.

The chromatographic device can have a second alkylsilyl coating in direct contact with the alkylsilyl coating of Formula I. The second alkylsilyl coating has the Formula II

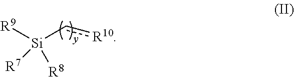

wherein R⁷, R⁸, and R⁹ are each independently selected from —NH(C₁-C₆)alkyl, —N[(C₁-C₆)alkyl]₂, (C₁-C₆)alkoxy, (C₁-C₆)alkyl, (C₁-C₆)alkenyl, OH, and halo; R¹⁰ is selected from (C₁-C₆)alkyl, —OR^{B}, —[O(C₁-C₃)alkyl]₁₋₁₀O(C₁-C₆)alkyl, —[O(C₁-C₃)alkyl]₁₋₁₀OH and phenyl. (C₁-C₆)alkyl is optionally substituted with one or more halo. The phenyl is optionally substituted with one or more groups selected from (C₁-C₃)alkyl, hydroxyl, fluorine, chlorine, bromine, cyano, —C(O)NH₂, and carboxyl. R^{B} is —(C₁-C₃)alkyloxirane, —(C₁-C₃)alkyl-3,4-epoxycyclohexyl, or —(C₁-C₄)alkylOH. The hashed bond to R¹⁰ represents an optional additional covalent bond between R¹⁰ and the carbon bridging the silyl group to form an alkene, provided y is not 0. y is an integer from 0 to 20.

In one aspect, y in Formula II is an integer from 1 to 15. In another aspect, y in Formula II is an integer from 1 to 12. In another aspect, y in Formula II is an integer from 1 to 10. In another aspect, y in Formula II is an integer from 2 to 9.

In one aspect R¹⁰ in Formula II is methyl and y is as described above for Formula II or the preceding paragraph.

In one aspect, R⁷, R⁸, and R⁹ in Formula II are each the same, wherein R¹⁰ and y are as described above. In one aspect, R⁷, R⁸, and R⁹ are each halo (e.g., chloro) or (C₁-C₆)alkoxy such as methoxy, wherein R¹⁰ and y are as described above.

In one aspect, y in Formula II is 9, R¹⁰ is methyl, and R⁷, R⁸, and R⁹ are each ethoxy or chloro.

In one aspect, the coating of the formula II is n-decyltrichlorosilane, (3-glycidyloxypropyl)trimethoxysilane (GPTMS), (3-glycidyloxypropyl)trimethoxysilane (GPTMS) followed by hydrolysis, 2-(3,4-epoxycyclohexyl)ethyltrimethoxysilane, trimethylchlorosilane, trimethyldimethyaminosilane, methoxy-polyethyleneoxy(3)silane propyltrichlorosilane, propyltrimethoxysilane, (heptadecafluoro-1,1,2,2-tetrahydrodecyl)tris(dimethylamino)silane, (heptadecafluoro-1,1,2,2-tetrahydrodecyl)trischlorosilane, (heptadecafluoro-1,1,2,2-tetrahydrodecyl)trimethoxysilane vinyltrichlorosilane, vinyltrimethoxysilane, allyltrichlorosilane, 2-[methoxy(polyethyleneoxy)3propyl]trichlorosilane, 2-[methoxy(polyethyleneoxy)3propyl]trimethoxysilane, or 2-[methoxy(polyethyleneoxy)3propyl]tris(dimethylamino)silane.

The alkylsilyl coating of Formula I and II can have a contact angle of at least about 15°. In some embodiments, the alkylsilyl coating of Formula I and II can have a contact angle of less than or equal to 105°. The contact angle can be less than or equal to about 115°. In other embodiments, the contact angle can be less than or equal to about 90°. In some embodiments, the contact angle of the alkylsilyl coating of Formula I and II is between about 150 to about 115°. For example, the contact angle of the alkylsilyl coating of Formula I and II can be about 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, or 115°.

The thickness of the multi-layered alkylsilyl coating can be at least about 100 Å. For example, the thickness can be between about 100 Å to about 1600 Å. The thickness of the multi-layered alkylsilyl coating for Formal I can be about 100 Å, 200 Å, 300 Å, 400 Å, 500 Å, 600 Å, 700 Å, 800 Å, 900 Å, 1000 Å, 1100 Å, 1200 Å, 1300 Å, 1400 Å, 1500 Å or 1600 Å.

In one aspect, the alkylsilyl coating of Formula I is bis(trichlorosilyl)ethane or bis(trimethoxysilyl)ethane and the alkylsilyl coating of Formula II is (3-glycidyloxypropyl)trimethoxysilane. In another aspect, the alkylsilyl coating of Formula I is bis(trichlorosilyl)ethane or bis(trimethoxysilyl)ethane and the alkylsilyl coating of Formula II is (3-glycidyloxypropyl)trimethoxysilane followed by hydrolysis. In one aspect, the alkylsilyl coating of Formula I is bis(trichlorosilyl)ethane or bis(trimethoxysilyl)ethane and the alkylsilyl coating of Formula II is n-decyltrichlorosilane. The alkylsilyl coating of Formula I can be bis(trichlorosilyl)ethane or bis(trimethoxysilyl)ethane and the alkylsilyl coating of Formula II can be trimethylchlorosilane or trimethyldimethyaminosilane. In one aspect, the alkylsilyl coating of Formula I is bis(trichlorosilyl)ethane or bis(trimethoxysilyl)ethane and the alkylsilyl coating of Formula II is methoxy-polyethyleneoxy(3) propyl tricholorosilane or methoxy-polyethyleneoxy(3) propyl trimethoxysilane.

Exemplary coatings with their respective approximate thickness and contact angle are provided in Table 1.

TABLE 1

| | Alternative | Approximate | Approximate |
|---|---|---|---|
| | Coating | Thickness | Contact |
| Vapor Deposited Material | Abbreviation | of Product | Angle |
| bis(trichlorosilyl)ethane or | C₂ | 500 Å | 35° |
| bis(trimethoxysilyl)ethane | | | |
| Annealed | Annealed C₂ | 500 Å | 95° |
| bis(trichlorosilyl)ethane or | | | |
| bis(trimethoxysilyl)ethane | | | |
| bis(trichlorosilyl)ethane or | C₂C₁₀ | 500 Å | 105° |
| bis(trimethoxysilyl)ethane as a | | | |
| first layer followed by n- | | | |
| decyltrichlorosilane as a second | | | |
| layer | | | |
| Annealed | Annealed | 500 Å | 105° |
| bis(trichlorosilyl)ethane or | C₂C₁₀ | | |
| bis(trimethoxysilyl)ethane as a | | | |
| first layer followed by annealed | | | |
| n-decyltrichlorosilane as a | | | |
| second layer | | | |

The first coating layer, C₂ shown below, is a layer according to Formula I, described above.

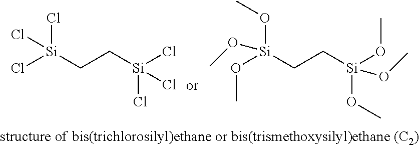

C₂C₁₀ is an example of a coating of Formula I and a second layer of Formula II. The structure of bis(trichlorosilyl)ethane or bis(trismethoxysilyl)ethane (C₂) is shown above. The structure of C₁₀ is shown below.

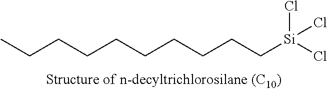

Alternatively, commercially available vapor deposition coatings can be used in the disclosed systems, devices, and methods, including but not limited to Dursan® and Dursox® (both commercially available from SilcoTek Corporation, Bellefonte, Pa.). The process for making is described in U.S. application Ser. No. 14/680,669, filed on Apr. 7, 2015, and entitled “Thermal Chemical Vapor Deposition Coated Article and Process,” which claims priority to and benefit of U.S. Provisional Application No. 61/976,789 filed Apr. 8, 2014. The contents of each application are incorporated herein by reference in their entirety.

In some examples, coating the flow path includes uniformly distributing the coating about the flow path, such that the walls defining the flow path are entirely coated. In some embodiments, uniformly distributing the coating can provide a uniform thickness of the coating about the flow path. In general, the coating uniformly covers the wetted surfaces such that there are no “bare” or uncoated spots.

The coatings described above can be used to create LBS and can tailor a fluidic flow path of a chromatography system for the separation of a sample. The coatings can be vapor deposited. In general, the deposited coatings can be used to adjust the hydrophobicity of internal surfaces of the fluidic flow path that come into contact with a fluid (i.e. wetted surfaces or surfaces coming into contact with the mobile phase and/or sample/analyte). By coating wetted surfaces of one or more components of a flow path within a chromatography system, a user can tailor the wetted surfaces to provide a desired interaction (i.e., a lack of interaction) between the flow path and fluids therein (including any sample, such as a sample containing B vitamins, within the fluid).

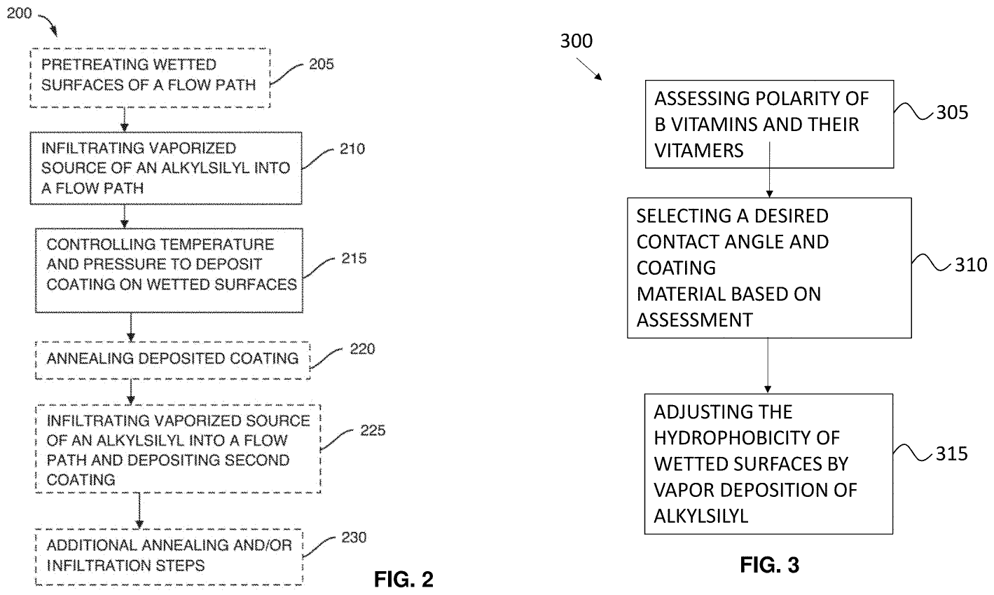

FIG. 2 is a flow chart illustrating method 200 for creating a LBS by tailoring a fluidic flow path for separation of a sample including B vitamins and their vitamers. The method has certain steps which are optional as indicated by the dashed outline surrounding a particular step. Method 200 can start with a pretreatment step (205) for cleaning and/or preparing a flow path within a component for tailoring. Pretreatment step 205 can include cleaning the flow path with plasma, such as oxygen plasma. This pretreatment step is optional.

Next, an infiltration step (210) is initiated. A vaporized source of an alkylsilyl compound is infiltrated into the flow path. The vaporized source is free to travel throughout and along the internal surfaces of the flow path. Temperature and/or pressure is controlled during infiltration such that the vaporized source is allowed to permeate throughout the internal flow path and to deposit a coating from the vaporized source on the exposed surface (e.g., wetted surfaces) of the flow path as shown in step 215. Additional steps can be taken to further tailor the flow path. For example, after the coating is deposited, it can be heat treated or annealed (step 220) to create cross linking within the deposited coating and/or to adjust the contact angle or hydrophobicity of the coating. Additionally or alternatively, a second coating of alkylsilyl compound (having the same or different form) can be deposited by infiltrating a vaporized source into the flow path and depositing a second or additional layers in contact with the first deposited layer as shown in step 225. After the deposition of each coating layer, an annealing step can occur. Numerous infiltration and annealing steps can be provided to tailor the flow path accordingly (step 230).

FIG. 3 provides a flow chart illustrating a method (300) of creating a LBS by tailoring a fluidic flow path for separation of a sample including a analyte, such as B vitamins and their vitamers. The method can be used to tailor a flow system for use in isolating, separating, and/or analyzing B vitamins and their vitamers. In step 305, B vitamins and their vitamers are assessed to determine polarity. Understanding the polarity will allow an operator to select (by either look up table or make a determination) a desired coating chemistry and, optionally, contact angle as shown in step 310.

In some embodiments, in addition to assessing the polarity of B vitamins and their vitamers, the polarity of a stationary phase to be used to separate the B vitamins and their vitamers (e.g., stationary phase to be included in at least a portion of the fluidic flow path) is also assessed. A chromatographic media (e.g., stationary phase) can be selected based on metal-sensitive compounds, e.g., B vitamins and their vitamers, in the sample. Understanding the polarity of metal-sensitive compounds (e.g., B vitamins and their vitamers) and the stationary phase is used in certain embodiments by the operator to select the desired coating chemistry and contact angle in step 310. The components to be tailored can then be positioned within a chemical infiltration system with environmental control (e.g., pressure, atmosphere, temperature, etc.) and precursor materials are infiltrated into the flow path of the component to deposit one or more coatings along the wetted surfaces to adjust the hydrophobicity as shown in step 315. During any one of infiltration, deposition, and condition steps (e.g. annealing), coatings deposited from the infiltration system can be monitored and if necessary precursors and or depositing conditions can be adjusted if required allowing for fine tuning of coating properties.

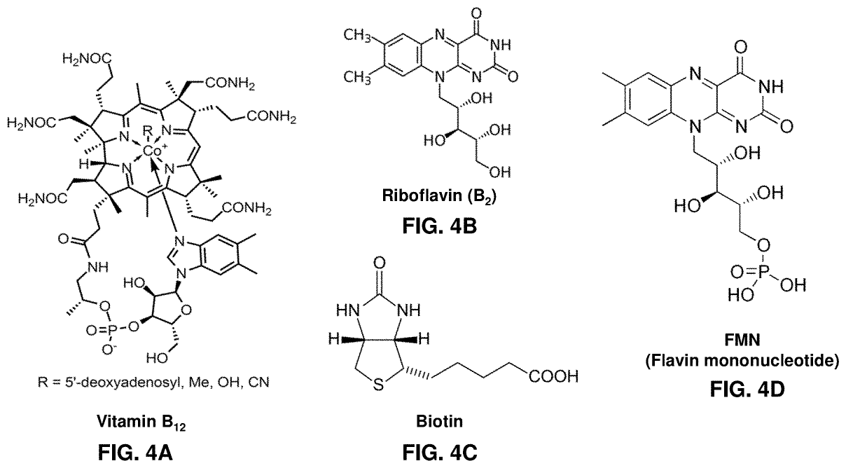

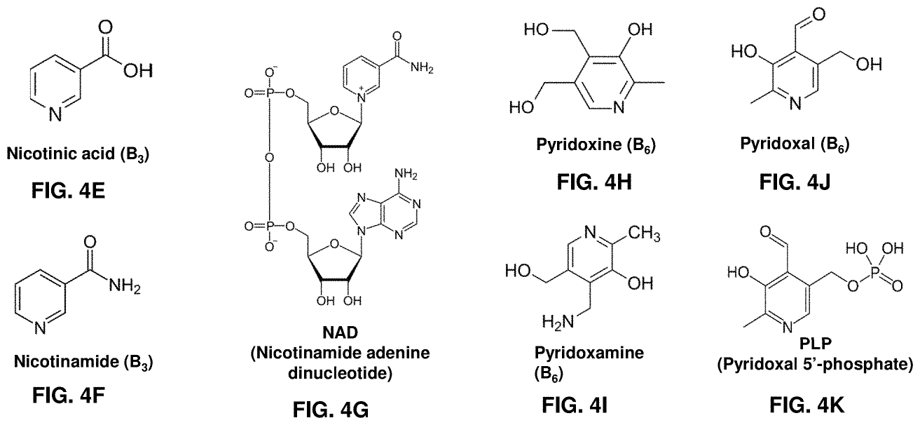

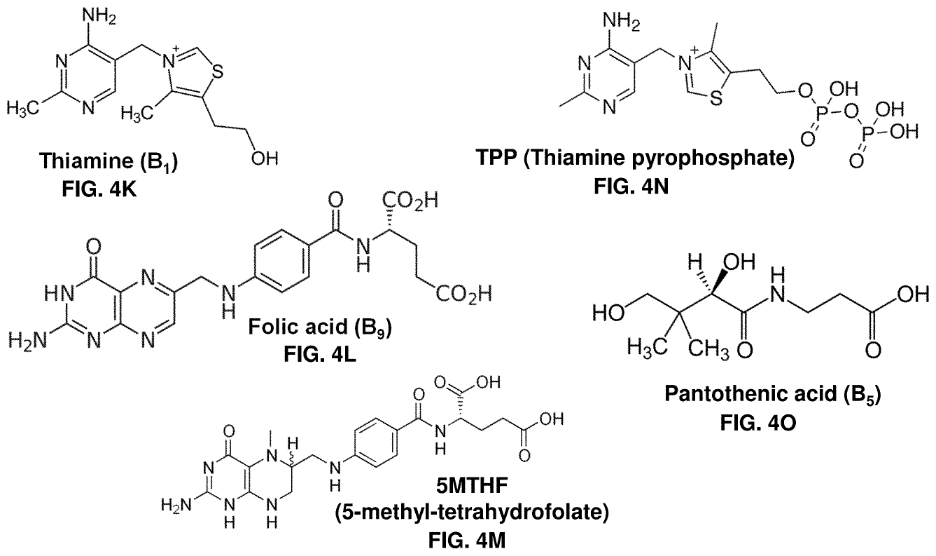

The structures of the B vitamins and their bioactive vitamers are shown in FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, FIG. 4N, and FIG. 4O. FIGS. 4A-4O show 18 compounds, namely; vitamin B₁₂, riboflavin (B₂), biotin, FMN (flavin mononucleotide), nicotinic acid (B₃), nicotinamide (B₃), nicotinamide adenine dinucleotide, pyridoxine (B₆), pyridoxamine (B₆), pyridoxal (B₆), PLP (Pyridoxal 5′-phosphate), thiamine (B₁), folic acid (B₉), 5MTHF (5-methyl-tetrahydrofolate), TPP (Thiamine pyrophosphate), and pantothenic acid (B₅), respectively.

Prior to any comparisons of coated column/hardware performance versus uncoated column/hardware performance for B vitamins and their vitamers, the following protocols were developed and used for sample preparation and analysis.

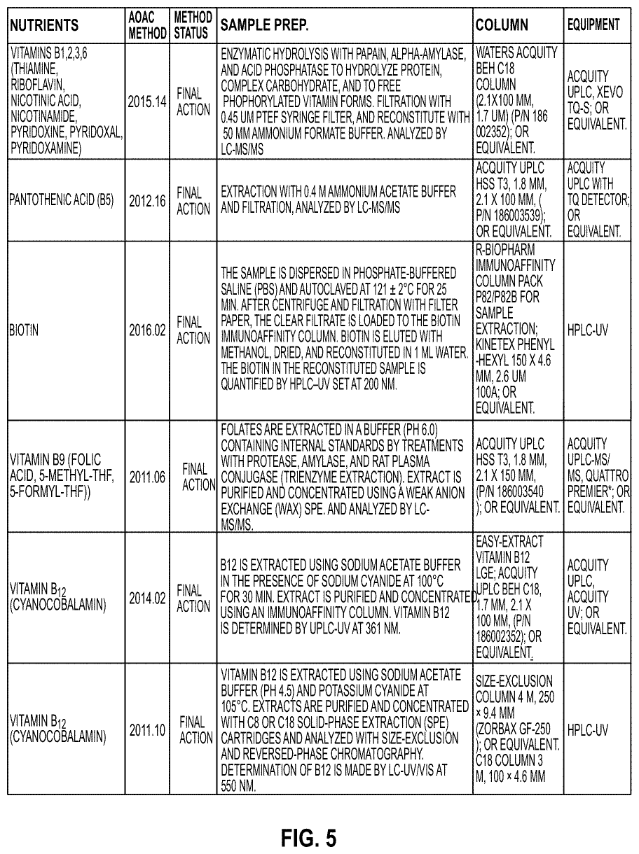

FIG. 5 displays the liquid chromatography conditions in the Association of Official Analytical Chemists (AOAC) Official Method for simultaneous determination of total vitamins B1, B2, B3, and B6 and other nutrients in matrices, such as infant formula and related nutritionals.

The present disclosure includes the simultaneous analysis of B vitamins. The examples included matrices of dietary supplements and energy drinks with B vitamins. B vitamin complex dietary supplements can contain multiple B vitamins, including different vitamers. Some dietary supplements include native, active, or coenzymated B vitamins, such as FMN and PLP. Energy drinks can contain vitamin B₁, B₂, B₃, B₅, B₆, B₇, B₉, B₁₂ and other ingredients (including natural product extracts).

Separate analyses are carried out for vitamins. For example, vitamin B₂ is analyzed with the fluorometric method; vitamins B₃, B₅, B₉, and B₁₂ are analyzed with a microbiological method; and vitamin B₆ with a liquid chromatography-fluorescence detector (LC-FLR) method. In contrast, the present disclosure analyzes B vitamins simultaneously, at least for simple matrices, such as dietary supplements and beverages. For complex samples, separate sample preparations for different B vitamins can be required. After the B vitamins are extracted from the complex sample matrices, a single LC method can be used to determine the vitamins of the sample.

### EXAMPLES

The examples studied the effects of the LBS on the analysis of B vitamins. Comparisons were made between the SOP configuration (as defined below) and the LBS configuration (as defined below) in peak area, peak height, LOQ, peak shape, carry-over, and response factor

Configurations—(both commercially available from Waters Technologies Corporation, Milford, Mass., USA):

SOP: ACQUITY UPLC™ BEH™ C₁₈ Column in ACQUITY UPLC H Class Plus System

LBS: ACQUITY™ Premier BEH C₁₈ Column in ACQUITY Premier System

Significant differences were observed between the SOP and LBS configurations. As compared to the SOP configuration, using the LBS configuration produced better sensitivity and no carry-over.

Sample preparation for the samples included sample preparation for dietary supplements and for energy drinks. For dietary supplements, 50 milligrams (recorded to 0.01 mg) of capsule content (fine powder), or 50 μL of liquid dietary supplement, were dissolved in 200 ml water in amber volumetric flasks. For powder samples, the samples were sonicated 5-10 seconds. The samples stood in darkness for at least 2 hours to ensure dissolution. The sample solutions were then filtered with 0.45-micron Glass MicorFiber (GMF) syringe filter. The first 2 ml of the filtrate were discarded and then 4 ml of the filtrate were collected. The analysis solutions were prepared by mixing the sample filtrates with water at different dilution ratios (95/100, 10/100, 1/100 v/v sample/total volume). Internal standard (IS) solution was also added into each analysis solution. For energy drinks, samples were sonicated to remove carbonation and filtered through a 0.45 micron GMF syringe membrane filter. The filtrate was then diluted with water at different dilution ratios (90/100, 2/100 v/v sample/total volume) prior to analysis. IS solution was added into each analysis solution as well.

The following experimental conditions were used for the examples.

LC System Waters™ ACQUITY UPLC H Class Plus System (SOP configuration); ACQUITY Premier System (LBS configuration). The LBS configuration

LC Column Waters ACQUITY UPLC BEH C₁₈ Column (1.7 μm, 2.1×100 mm) (SOP configuration); ACQUITY Premier BEH C₁₈ Column (1.7 μm, 2.1×100 mm) (LBS configuration)

The SOP configuration is a system and column with stainless steel and no coating. The LBS configuration is a system and column with stainless steel and a C₂ coating.

Mobile phases:

A: 20 mM ammonium formate in water (pH 5.0)

B: 100% methanol

Column temperature: 40° C.

Autosampler temperature: 5° C.

Injection volume: 2 μL

Gradient program:

| Time | Flow | A | B |
|---|---|---|---|
| (min) | (ml/min) | (%) | (%) |
| Initial | 0.35 | 99 | 1 |
| 0.50 | 0.35 | 99 | 1 |
| 2.50 | 0.35 | 92 | 8 |
| 5.00 | 0.35 | 10 | 90 |
| 6.00 | 0.35 | 10 | 90 |
| 6.10 | 0.35 | 99 | 1 |

MS system: Xevo TQ-S Micro System

MS system settings:

| Polarity | ES+ |
|---|---|
| Capillary (kV) | 1.4 |
| Cone (V) | 70 |
| Source Temperature (° C.) | 150 |
| Desolvation Temperature (° C.) | 350 |
| Cone Gas Flow (L/Hr) | 100 |
| Desolvation Gas Flow (L/Hr) | 650 |

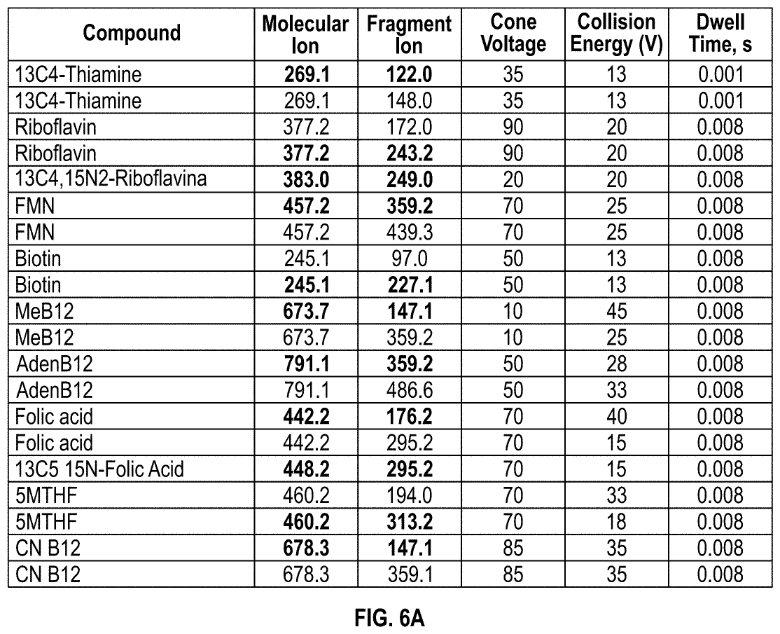

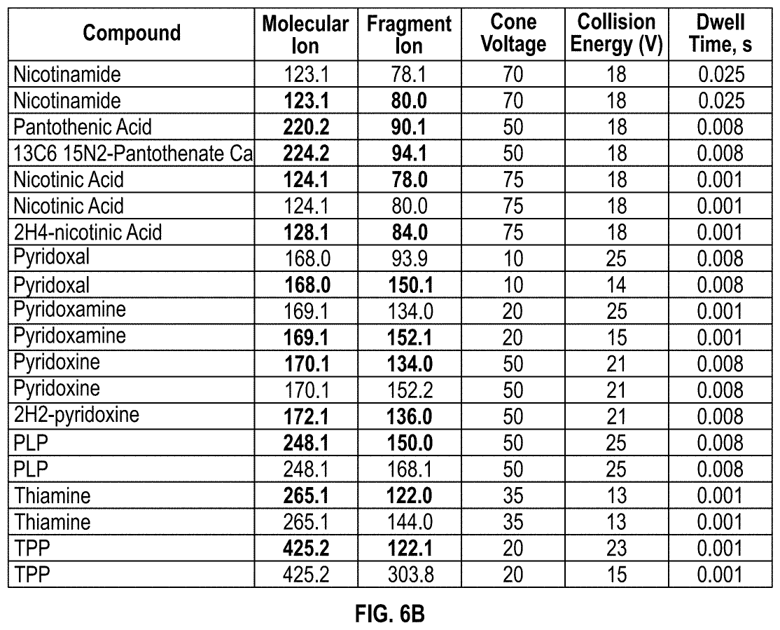

FIG. 6A and FIG. 6B display the multiple reaction monitoring (MRM) transitions and MS detection parameters for 18 vitamins, including the additional vitamins B₅, B₉, B₁₂ and biotin, that were used for the Xevo TQ-S Micro MS System. Two LC system setups were used for comparison, one included an ACQUITY Premier System and an ACQUITY Premier BEH C₁₈ (1.7 m, 2.1×100 mm) Column with a C₂ coating (referred to as the LBS setup/configuration), and the other one included an ACQUITY UPLC H Class Plus System and an ACQUITY UPLC BEH C₁₈ (1.7 μm, 2.1×100 mm) Column (referred to as the SOP setup/configuration). An alkylsilyl coating with C₂ was used in the LBS setup while the stainless-steel parts were used in the SOP setup. These two systems were coupled to the same Xevo TQ-S Micro MS System sequentially to minimize instrumental variables in the comparison study.

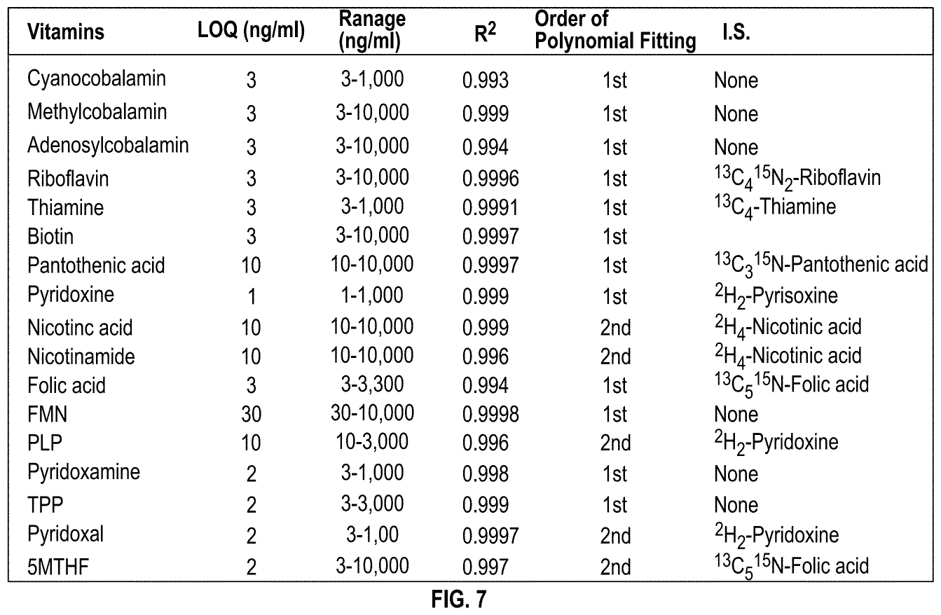

FIG. 7 displays a table of calibration data, in accordance with examples of the present disclosure. The table of calibration data includes for each vitamin the limit of quantitation (LOQ) (ng/ml), range (ng/ml), R², order of polynomial fitting, and internal standards.

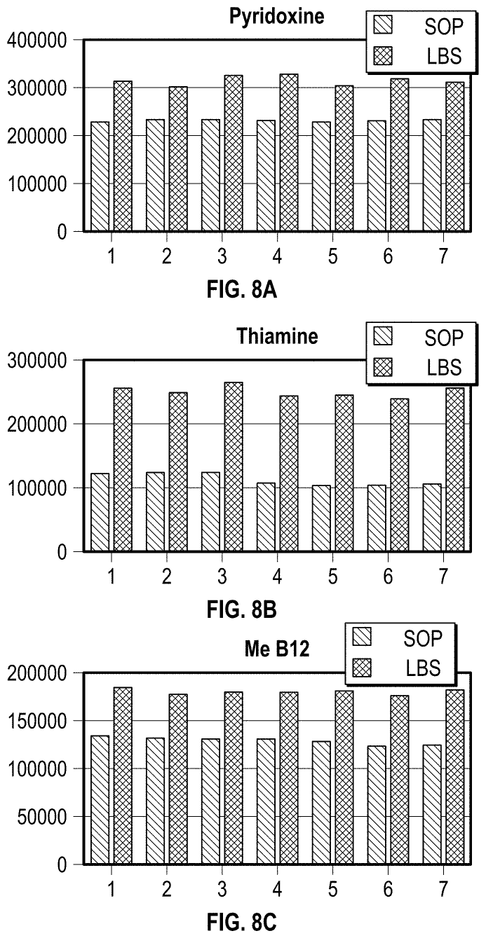

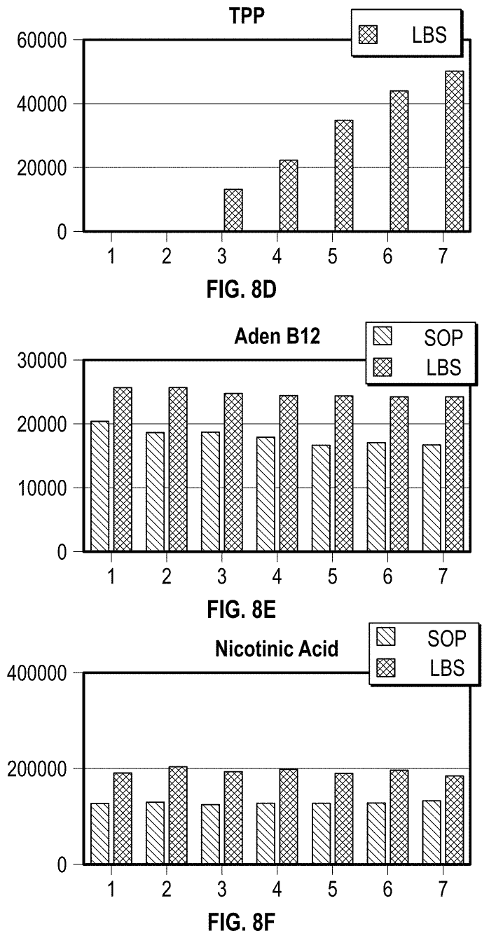

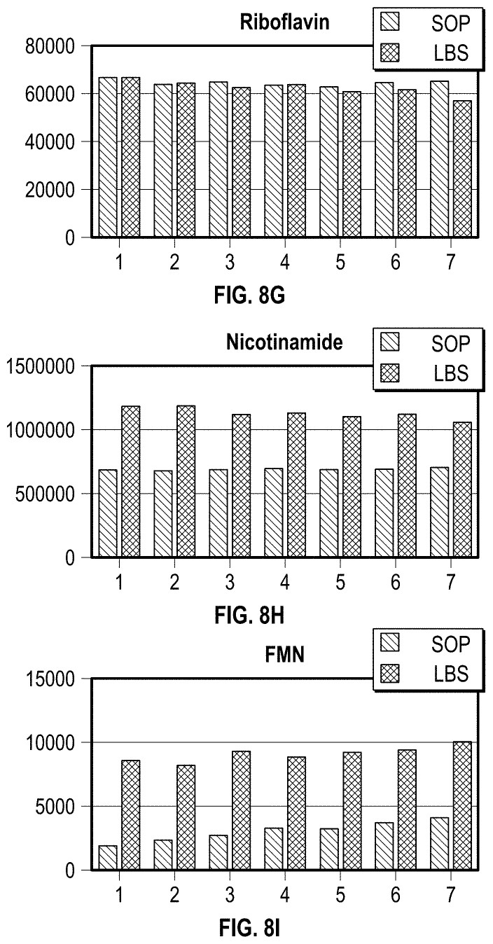

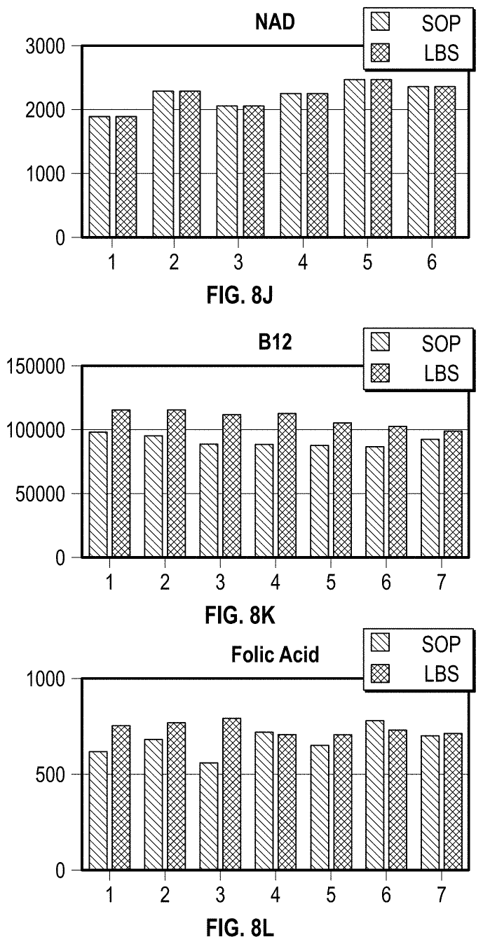

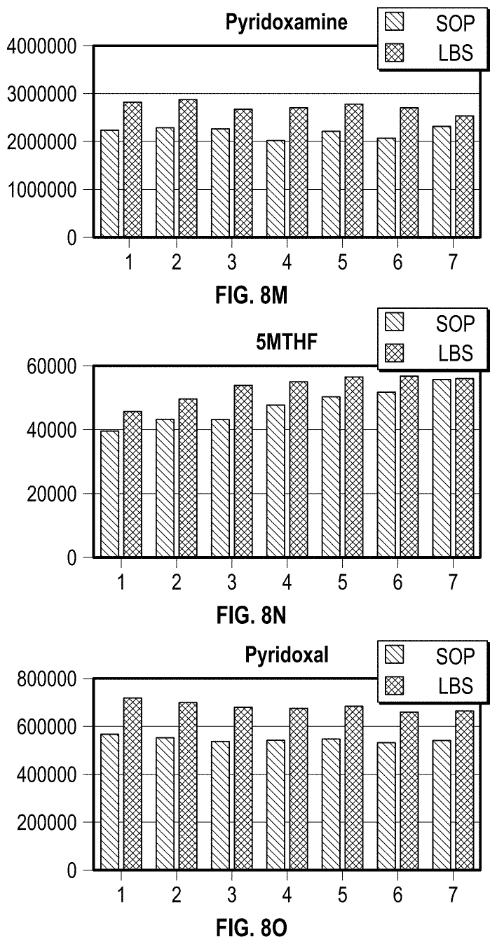

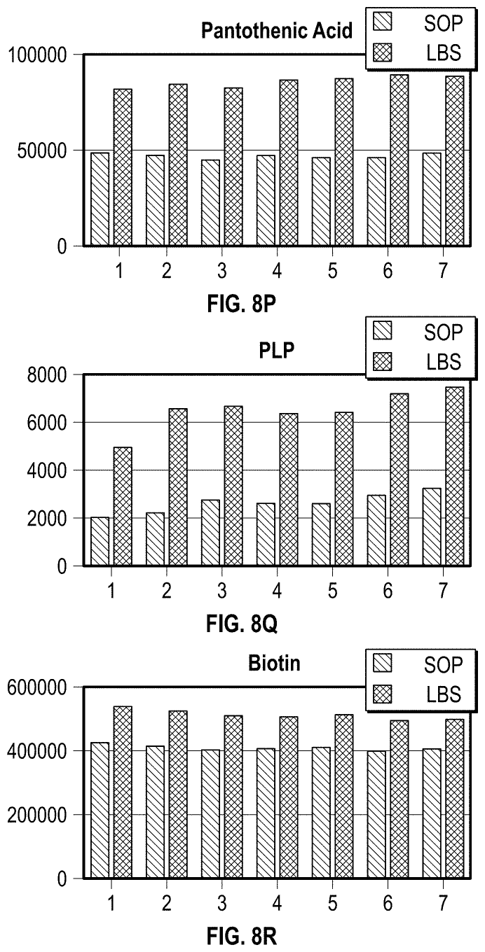

FIGS. 8A-8R show comparison plots of the vitamin peak areas from 7 replicate injections of a standard mix solution (at a concentration of 1 μg/mL) in the SOP configuration and the LBS configuration. Peak areas from standard surface (SOP configuration) are plotted together with those from LBS surface (LBS configuration). The B vitamins and vitamers include pyridoxine (FIG. 8A), thiamine (FIG. 8B), MeB12 (methyl cobalamine) (FIG. 8C), TPP (Thiamine pyrophosphate) (FIG. 8D), Aden B12 (adenosyl cobalamin)(FIG. 8E), nicotinic acid (FIG. 8F), riboflavin (FIG. 8G), nicotinamide (FIG. 8H), FMN (Flavin mononucleotide)(FIG. 8I), nicotinamide adenine dinucleotide (NAD)(FIG. 8J), B12 (FIG. 8K), folic acid (FIG. 8L), pyridoxamine (FIG. 8M), 5MTHF (50methyl-tetrahydrofolate)(FIG. 8N), pyridoxal (FIG. 8O), pantothenic acid (FIG. 8P), PLP (Pyridoxal 5′-phosphate)(FIG. 8Q), and biotin (FIG. 8R). Increased responses of B vitamins were observed using the LBS configuration. LC systems were flushed and cleaned prior to the comparison study. The data were obtained when the systems and columns were new for both SOP configuration and the LBS configuration (no B vitamins had ever been injected onto the systems). After initial LC equilibration and injections of blanks (solvent), consecutive injections of the same standard mix solution (concentrations were at 1 ppm, or 1 μg/ml for each standard) were compared. The comparison was of LC-MS chromatographic peak areas of vitamin B₁, B₂, B₃, B₅, B₆, B₇, B₉, B₁₂ and their vitamers in the initial 7 injections of the same standard solution using the LBS configuration and the SOP configuration.

Higher peak intensities and larger peak areas were observed for majority of the 18 vitamins using the LBS configuration. One can see that greater or the same responses (peak areas) were obtained in the LBS configuration for all 18 vitamins. Significant differences for TPP, Thiamine, FMN (flavin mononucleotide), PLP, and pantothenic acid were observed. The peak areas of these compounds from the LBS configuration were significantly larger than those peak areas from the SOP configuration. TPP did not show any peak in the SOP configuration under the conditions of the examples. Other compounds, such as cyanocobalamine (B12), methyl-cobalamine (MeB12), adenosyl-cobalamin (Aden B12), nicotinic acid, nicotinamide, pyridoxine, pyridoxaminde, pyridoxal, biotin, folic acid, and 5-methyl-tetrahydrofolate (5MTHF) show larger peak areas in the LBS configuration than those in the SOP configuration.

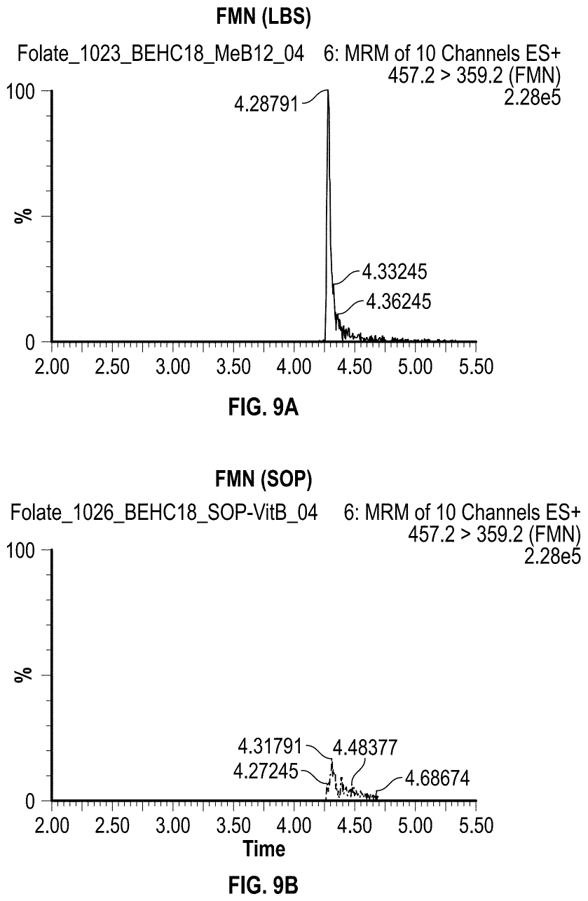

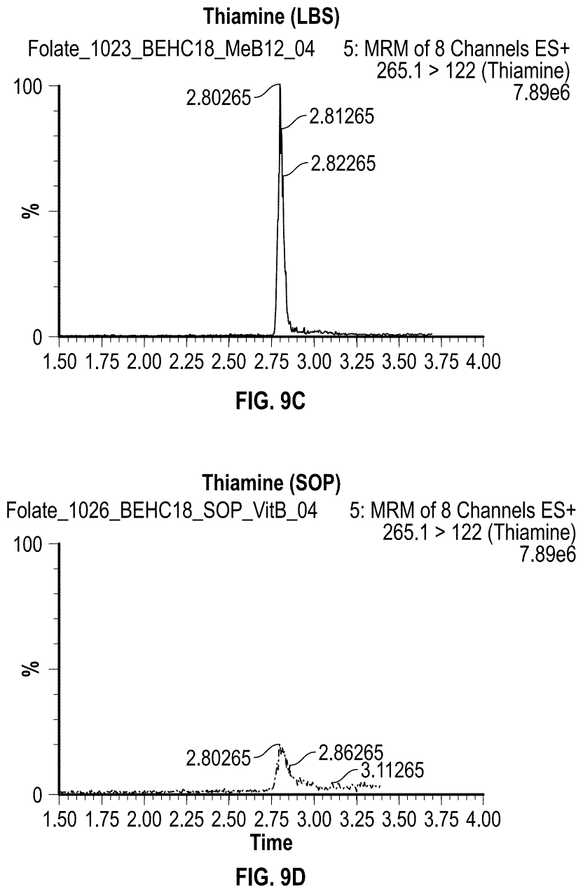

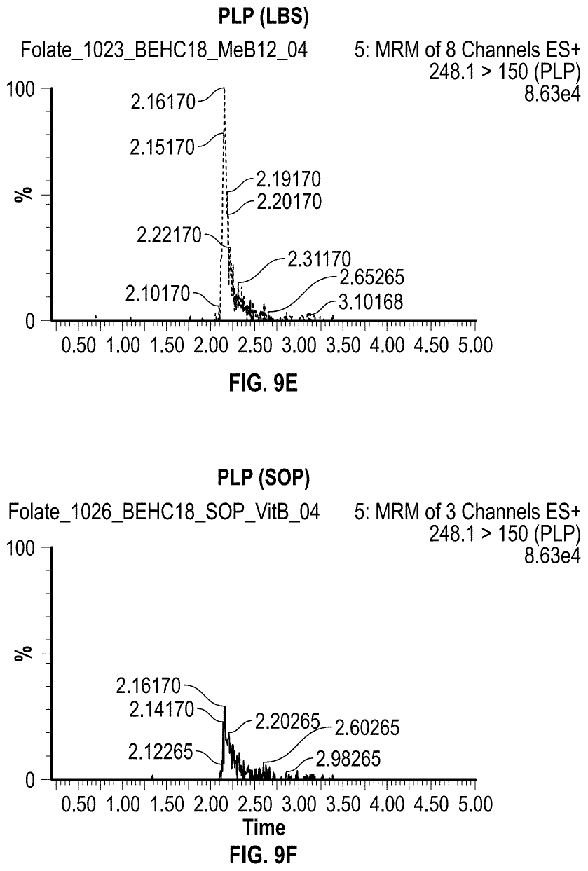

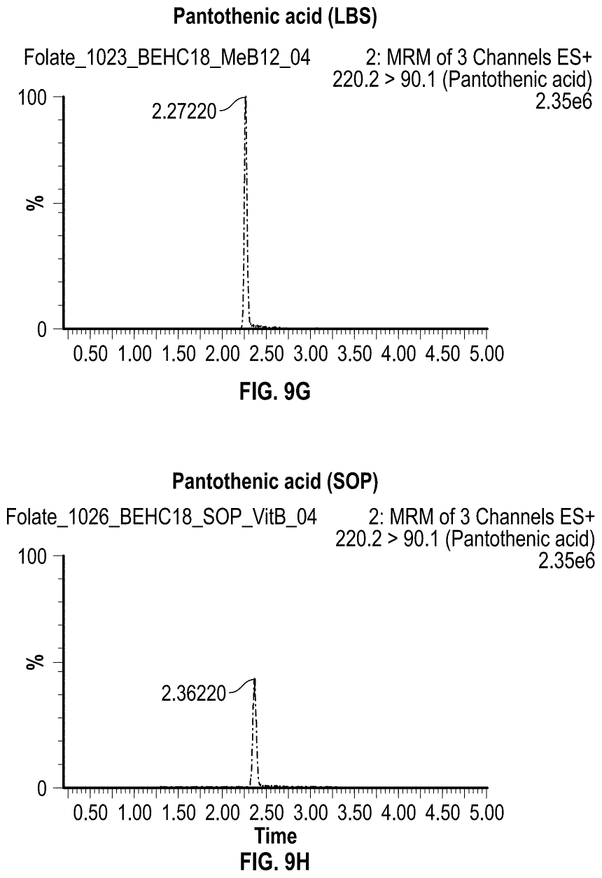

FIG. 9A, FIG. 9B, FIG. 9C, FIG. 9D, FIG. 9E, FIG. 9F, FIG. 9G, and FIG. 9H compare the chromatograms for the LBS configuration and the SOP configuration for flavin mononucleotide (FMN), thiamine (B₁), pyridoxal 5′-phosphate (PLP), and pantothenic acid (B₅), respectively, in accordance with the present disclosure. FIG. 9A, FIG. 9C, FIG. 9E, and FIG. 9G display the chromatograms for the LBS configuration for flavin mononucleotide (FMN), thiamine (B₁), pyridoxal 5′-phosphate (PLP), and pantothenic acid (B₅), respectively, in accordance with the present disclosure. FIG. 9B, FIG. 9D, FIG. 9F, and FIG. 9H display the chromatograms for the SOP configuration for flavin mononucleotide (FMN), thiamine (B₁), pyridoxal 5′-phosphate (PLP), and pantothenic acid (B₅), respectively, in accordance with the present disclosure. Vertical axes are the same scales for each compound comparison in FIG. 9A to FIG. 9H. The standard concentration was 1 ppm (μg/ml). The chromatograms of FIG. 9A, FIG. 9C, FIG. 9E, and FIG. 9G show the positive effect the LBS configuration has on peak area (peak intensity).

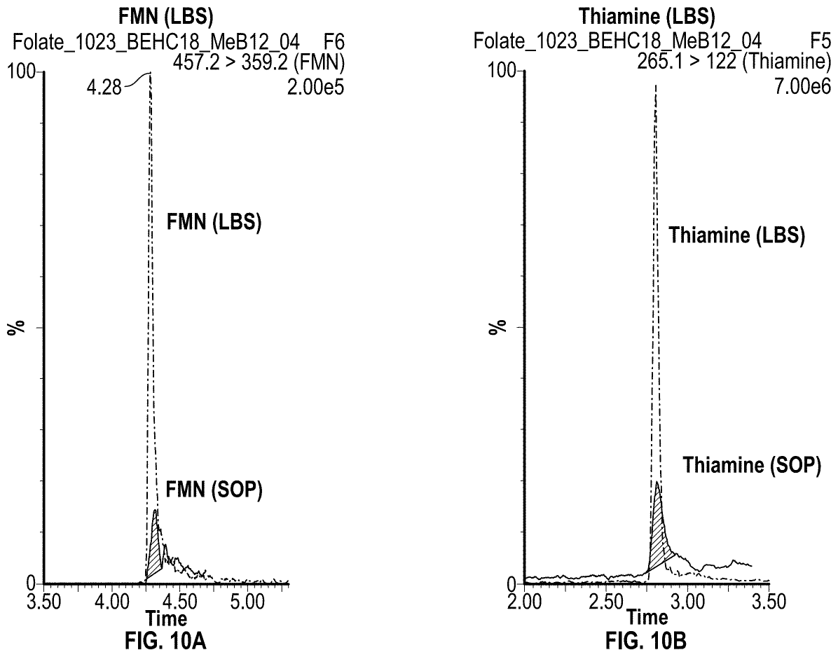

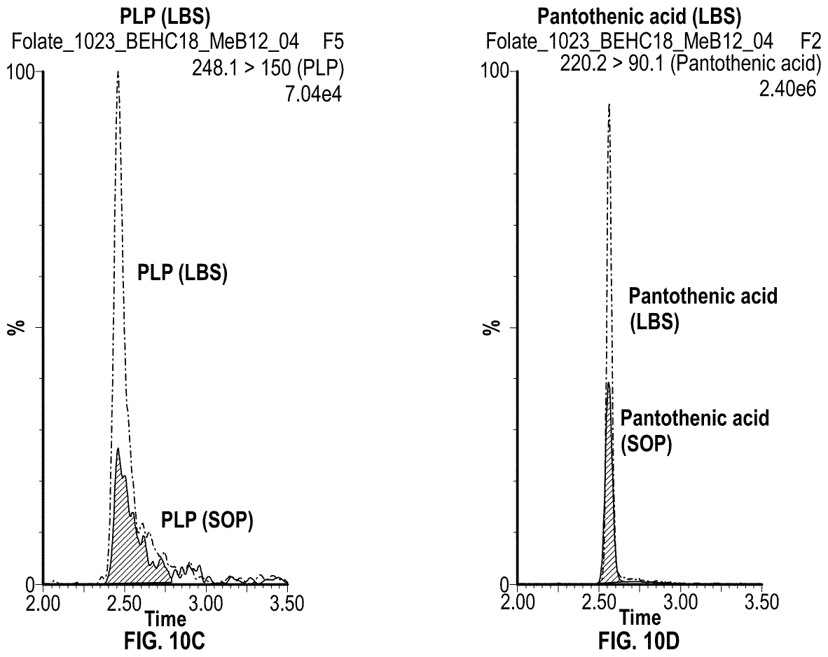

FIG. 10A, FIG. 10B, FIG. 10C, and FIG. 10D are comparisons of LC-MS chromatograms of FMN (FIG. 10A), Thiamine (FIG. 10B), PLP (FIG. 10C), and Pantothenic acid (FIG. 10D) from the initial injection in the same standard solution using LBS surfaces (LBS configuration) vs standard surfaces (SOP configuration). FIGS. 10A-10D display that the peak intensities were significantly increased using the LBS configuration. Also, narrower peaks and less peak tailing was observed for the thiamine (FIG. 10B) and PLP (FIG. 10C) peaks in the LBS configuration. The increase in response in LC-MS/MS analysis of B vitamins with the LBS configuration was still evident after extended use of the LC-MS system. Peak intensities from standard surface (solid filled peaks) are normalized to the peak intensities (not filled peak) from LBS.

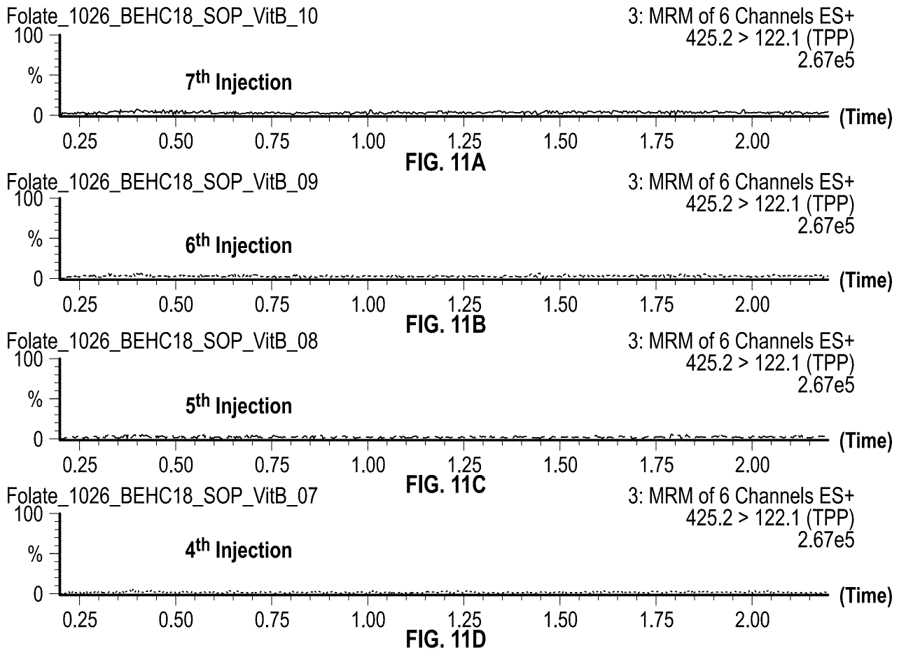

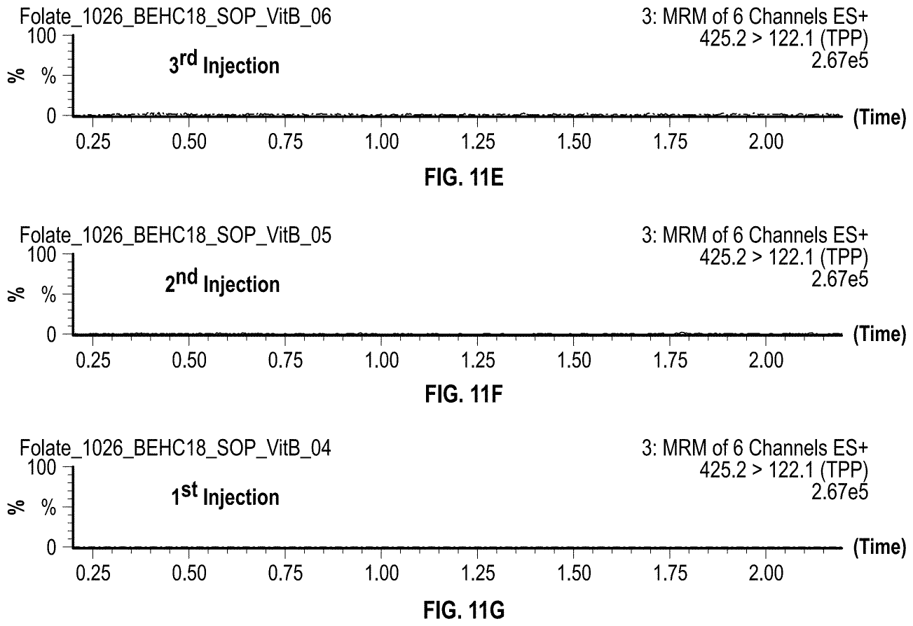

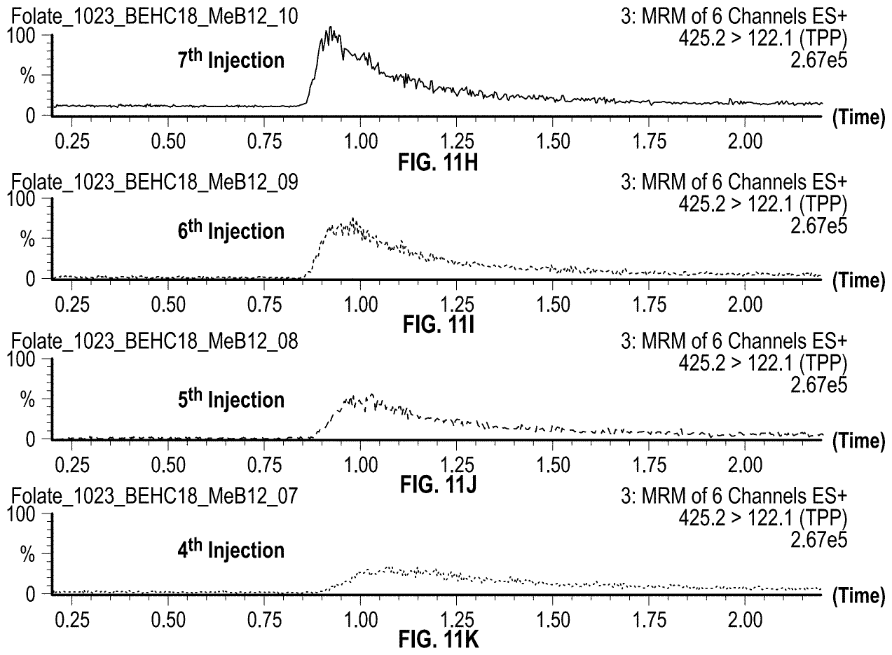

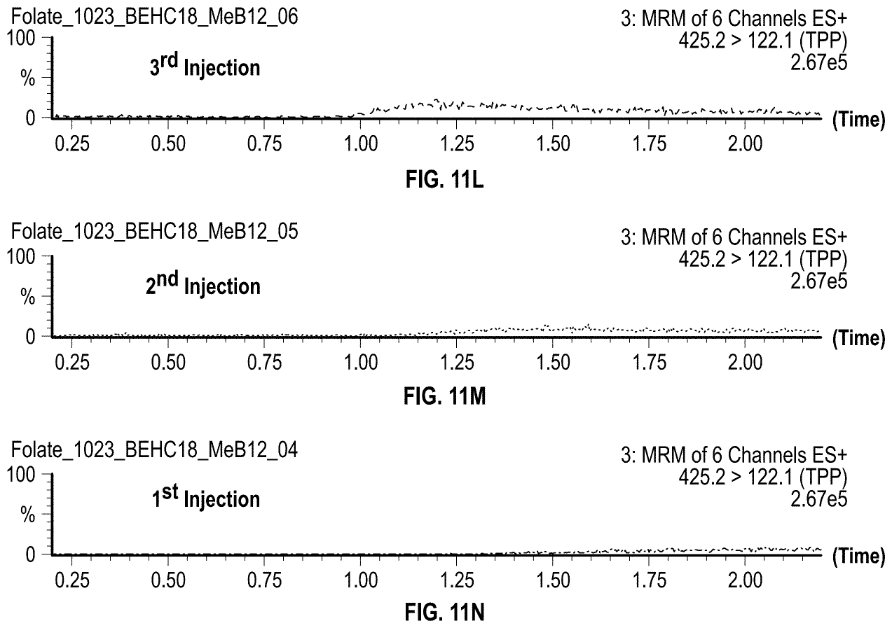

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L, FIG. 11M, and FIG. 11N compare thiamine pyrophosphate (TPP) chromatograms from initial seven injections for the SOP configuration and the LBS configuration, in accordance with the present disclosure. FIG. 11A, FIG. 11B, FIG. 11C, FIG. 11D, FIG. 11E, FIG. 11F and FIG. 11G display chromatograms from 7^{th}, 6^{th}, 5^{th}, 4^{th}, 3^{rd}, 2^{nd} and 1^{st} injections for the SOP configuration, respectively. FIG. 11H, FIG. 11I, FIG. 11J, FIG. 11K, FIG. 11L, FIG. 11M and FIG. 11N display chromatograms from 7^{th}, 6^{th}, 5^{th}, 4^{th}, 3^{rd} 2^{nd} and 1^{st} injections for the LBS configuration.

In the LBS configuration, as shown in FIG. 11H, FIG. 11I, FIG. 11J, FIG. 11K, FIG. 11L, FIG. 11M and FIG. 11N, the TPP peak is gradually formed in shape after initial injections. In contrast, the SOP configuration, as shown in FIG. 11A, FIG. 11B, FIG. 11C, FIG. 11D, FIG. 11E, FIG. 11F and FIG. 11G, displays no TPP peak. FIG. 11H, FIG. 11I, FIG. 11J, FIG. 11K, FIG. 11L, FIG. 11M and FIG. 11N displays how the LBS configuration helps alleviate the issue of surface adsorption of TPP. The MS probe has a metal surface that might contribute to the adsorption. The TPP peak in FIG. 11H, FIG. 11I, FIG. 11J, FIG. 11K, FIG. 11L, FIG. 11M and FIG. 11N changed during the consecutive injections. Without wishing to be bound by theory, the exposure history of the system and column to the TPP may have an effect in peak area and peak shape.

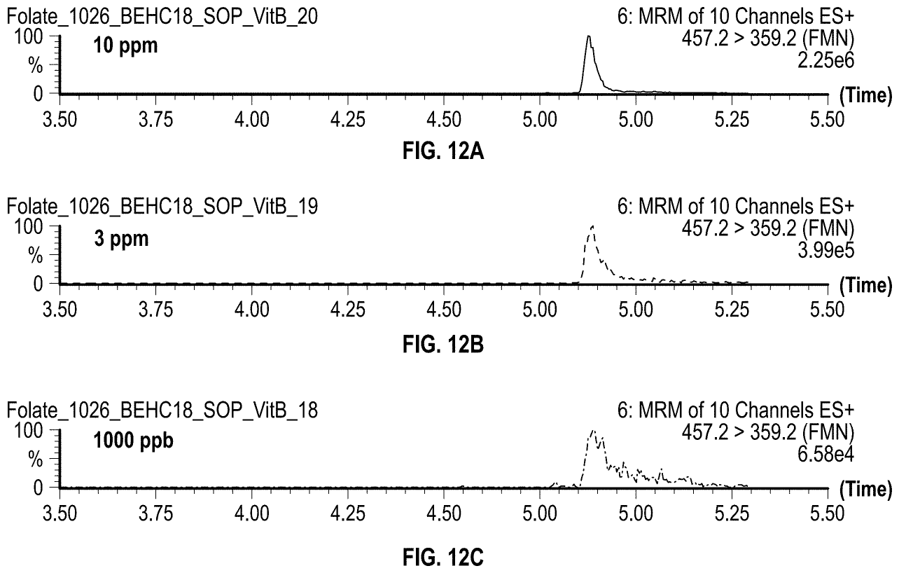

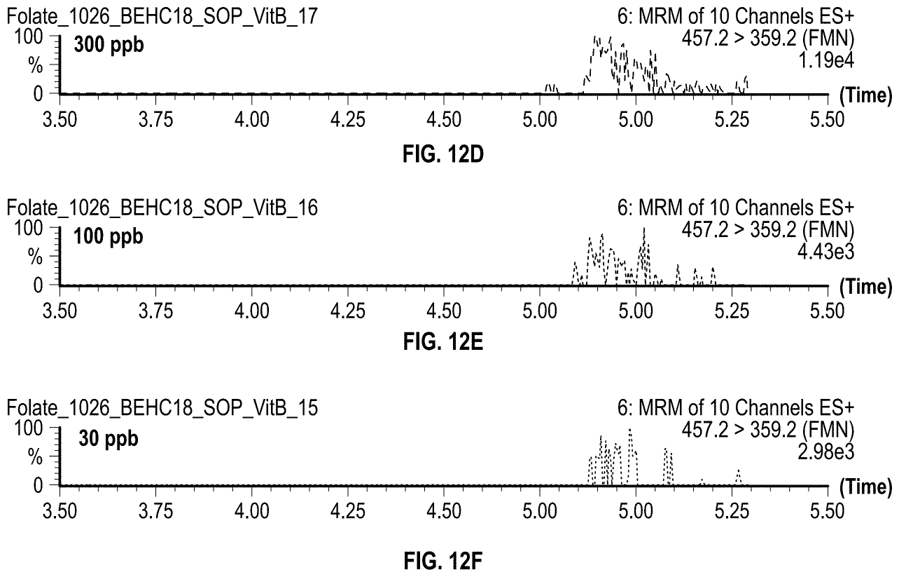

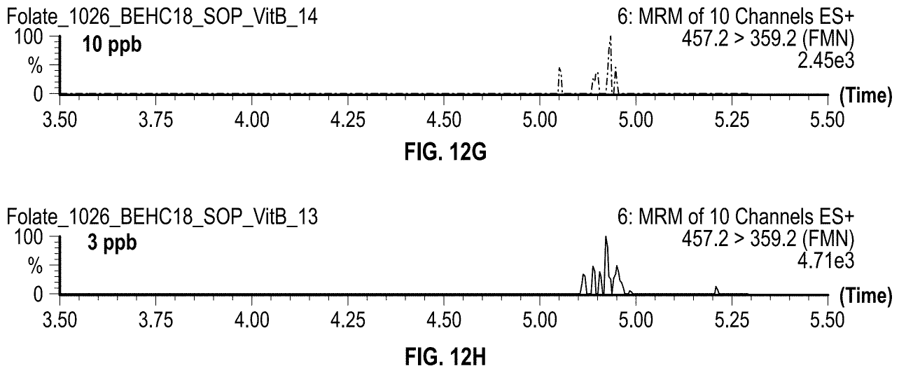

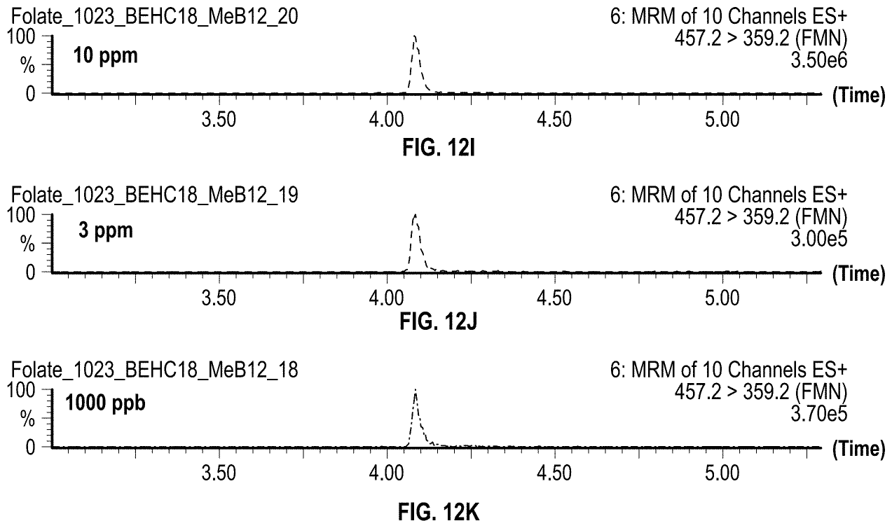

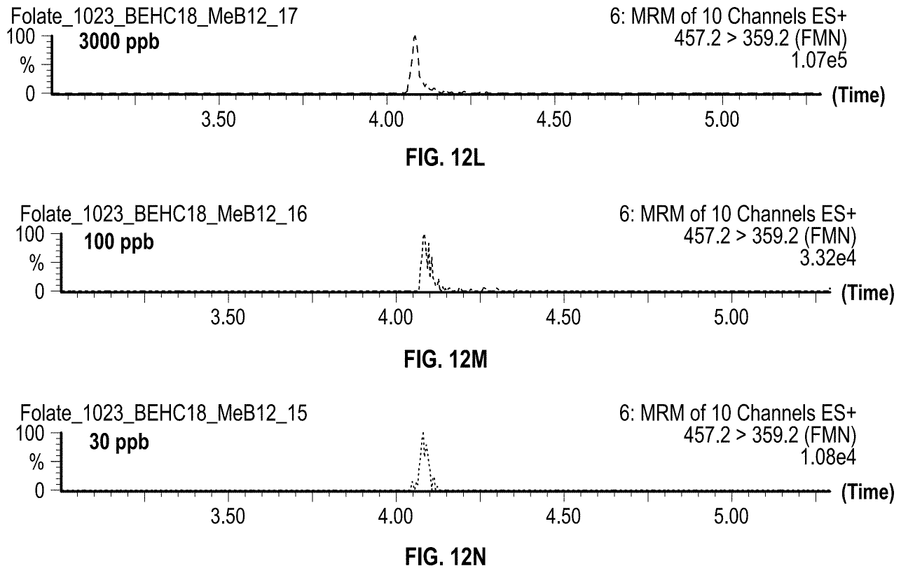

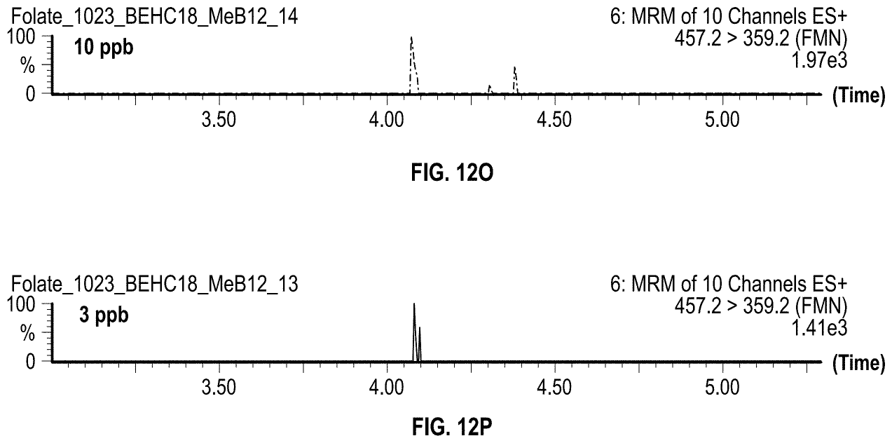

FIGS. 12A-12P compare the chromatograms of flavin mononucleotide (FMN) in standard solutions for the SOP configuration and the LBS configuration, in accordance with the present disclosure. FIG. 12A, FIG. 12B, FIG. 12C, FIG. 12D, FIG. 12E, FIG. 12F, FIG. 12G and FIG. 12H display chromatograms for 10 ppm, 3 ppm, 1000 ppb, 300 ppb, 100 ppb, 30 ppb, 10 ppb, and 3 ppb of flavin mononucleotide (FMN) concentrations, respectively, in standard solutions for the SOP configuration. FIG. 12I, FIG. 12J, FIG. 12K, FIG. 12L, FIG. 12M, FIG. 12N, FIG. 12O and FIG. 12P display chromatograms for 10 ppm, 3 ppm, 1000 ppb, 300 ppb, 100 ppb, 30 ppb, 10 ppb, and 3 ppb of flavin mononucleotide (FMN) concentrations, respectively, in standard solutions for the LBS configuration.

The standard concentration ranged from 3 ppb to 10 ppm. For the SOP configuration, as shown in FIGS. 12A-12H, the limit of quantitation (LOQ) was approximately 1000 ppb. For the LBS configuration, as shown in FIGS. 12I-12P, the LOQ was lower at approximately 100 ppb. In addition, peak shape was symmetric for the LBS configuration of FIGS. 12I-12P and the peak shape for the SOP configuration of FIGS. 12A-12H, showed tailing. A comparison of the results of FIGS. 12A-12H to FIGS. 12I-12PI, illustrates that using the LBS configuration enhanced sensitivity and peak shape.

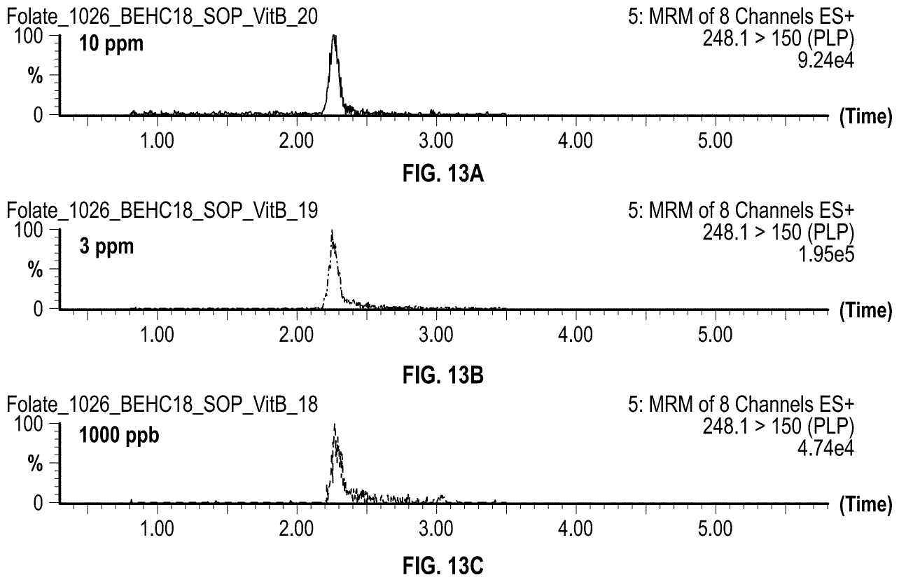

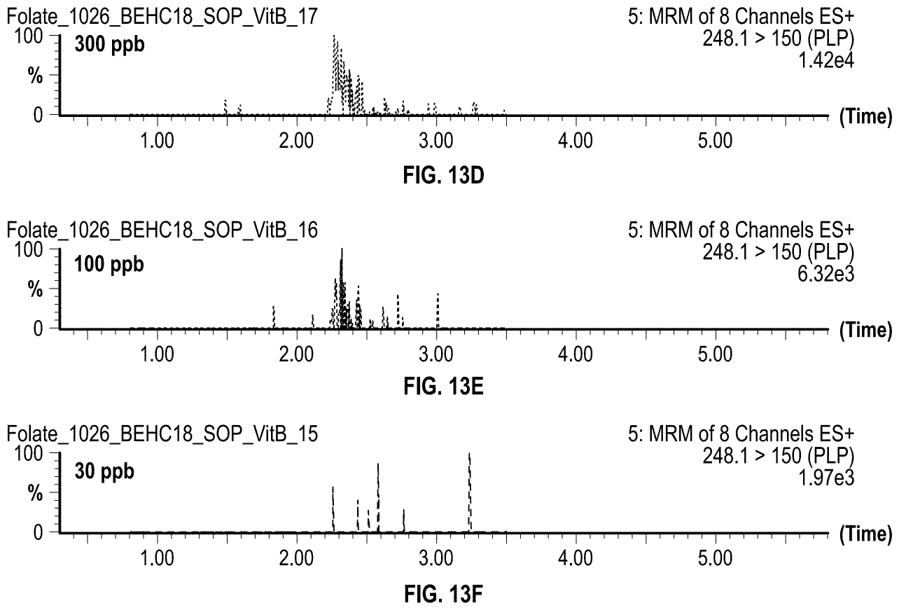

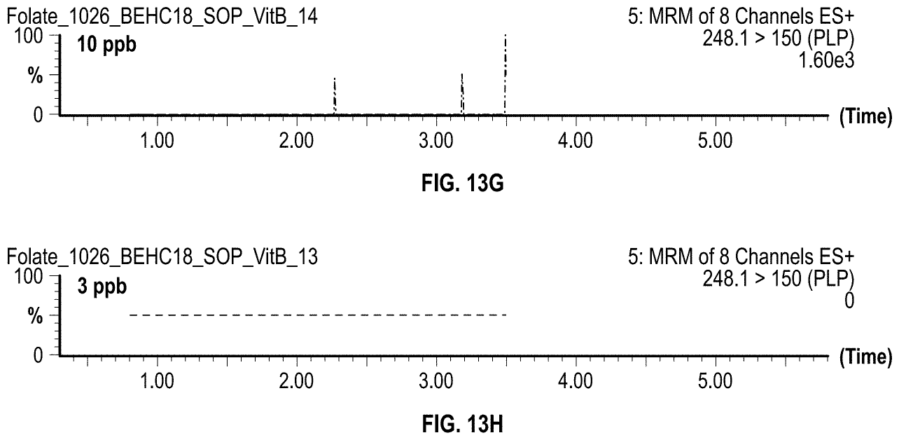

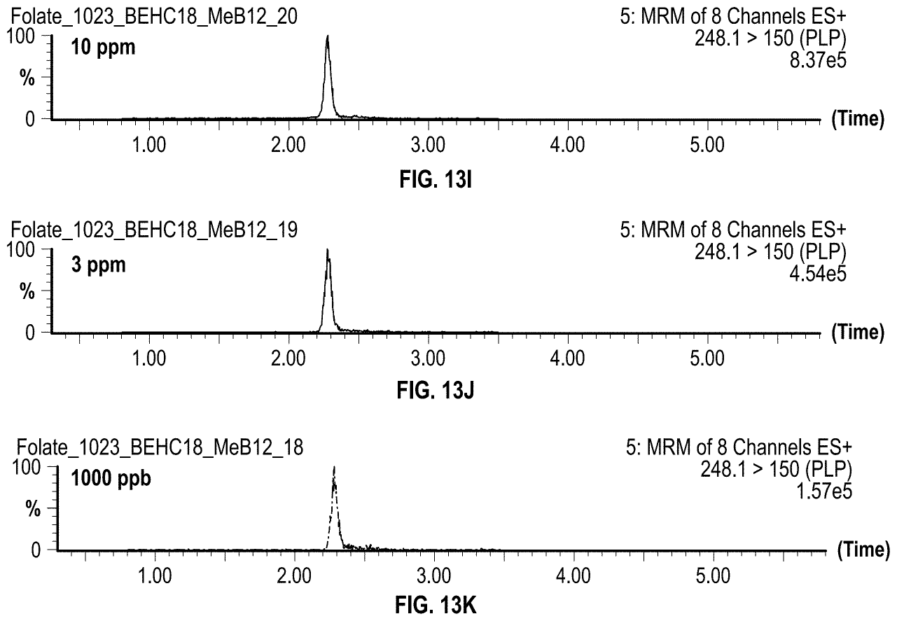

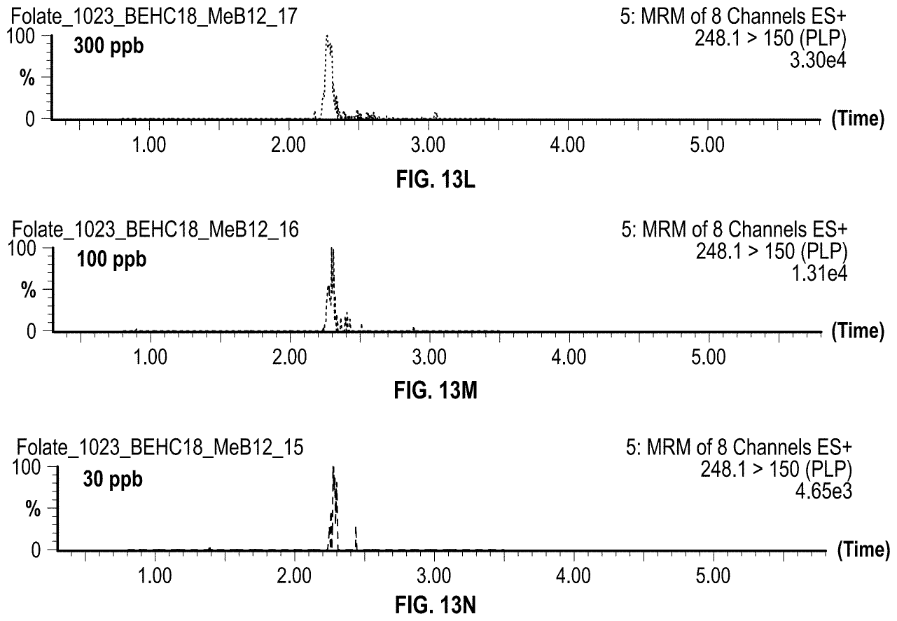

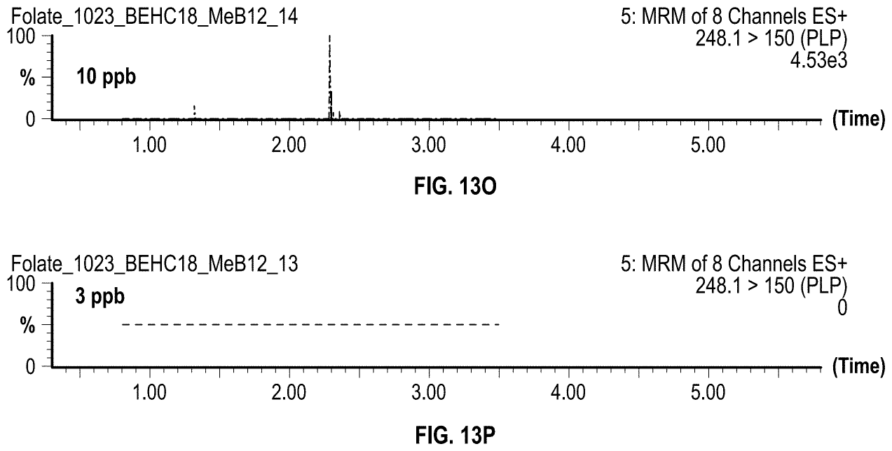

FIGS. 13A-13P compare the chromatograms of pyridoxal 5′-phosphate (PLP) in standard solutions for the SOP configuration and the LBS configuration, in accordance with the present disclosure. FIG. 13A, FIG. 13B, FIG. 13C, FIG. 13D, FIG. 13E, FIG. 13F, FIG. 13G and FIG. 13H display chromatograms for 10 ppm, 3 ppm, 1000 ppb, 300 ppb, 100 ppb, 30 ppb, 10 ppb, and 3 ppb of pyridoxal 5′-phosphate (PLP) concentrations, respectively, in standard solutions for the SOP configuration. FIG. 13I, FIG. 13J, FIG. 13K, FIG. 13L, FIG. 13M, FIG. 13N, FIG. 13O and FIG. 13P display chromatograms for 10 ppm, 3 ppm, 1000 ppb, 300 ppb, 100 ppb, 30 ppb, 10 ppb, and 3 ppb of pyridoxal 5′-phosphate (PLP) concentrations, respectively, in standard solutions for the LBS configuration.

Thus, FIGS. 13A-13H display chromatograms using the SOP configuration; whereas FIGS. 13I-13P display chromatograms using the LBS configuration. The standard concentration ranged from 3 ppb to 10 ppm. For the SOP configuration, as shown in FIGS. 13A-13H, the limit of quantitation (LOQ) was approximately 1000 ppb. For the LBS configuration, as shown in FIGS. 13I-13P, the LOQ was lower at approximately 300 ppb. In addition, peak shape was narrower for the LBS configuration of FIGS. 13I-13P than the SOP configuration of FIGS. 13A-13H. A comparison of the results of FIGS. 13A-13H to FIGS. 13I-13P, illustrates that using the LBS configuration enhanced sensitivity and peak shape.

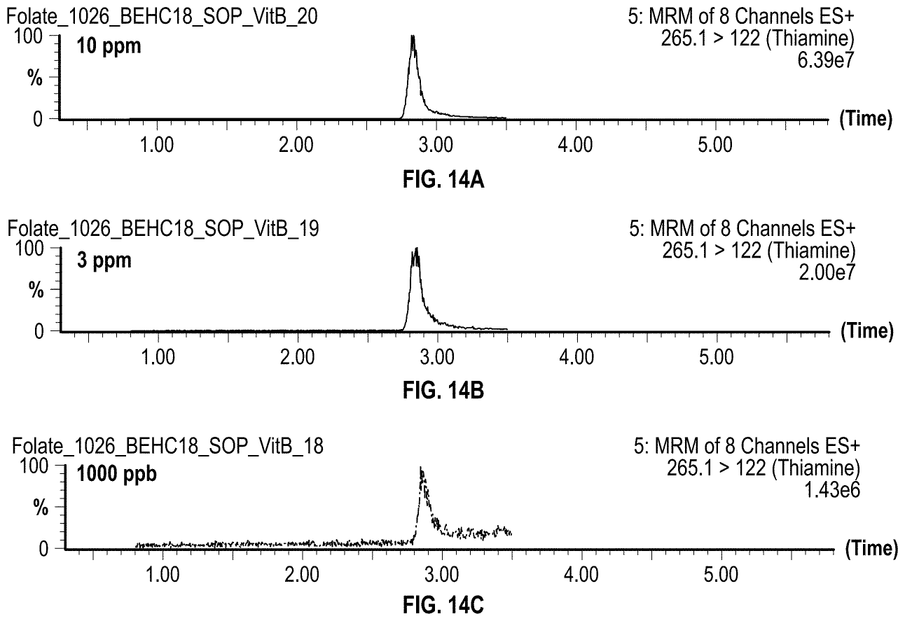

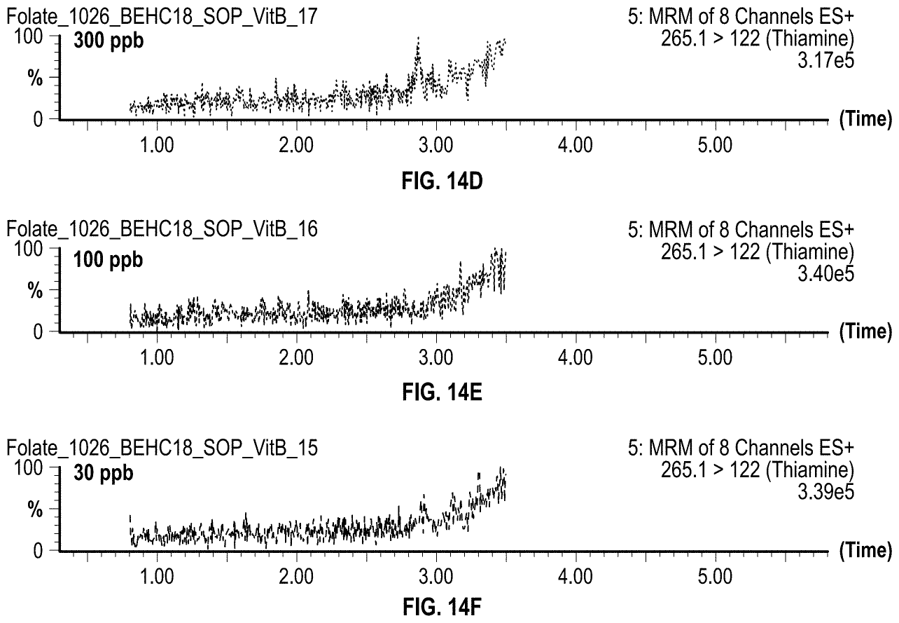

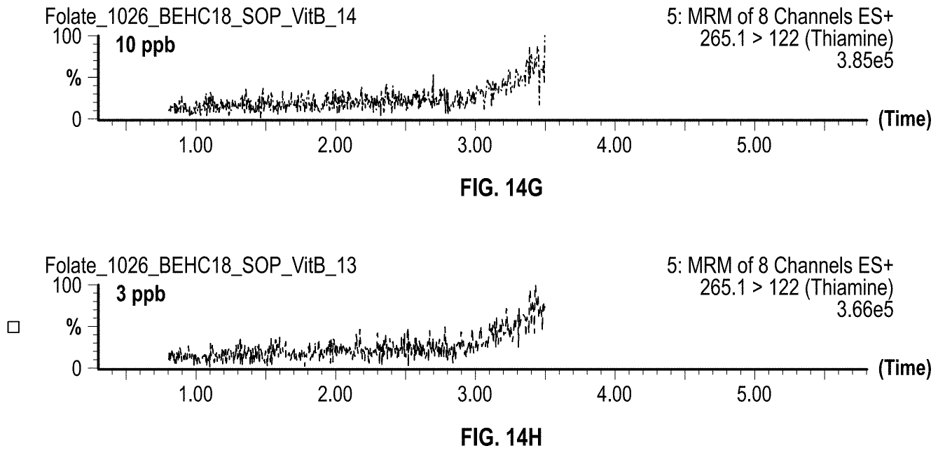

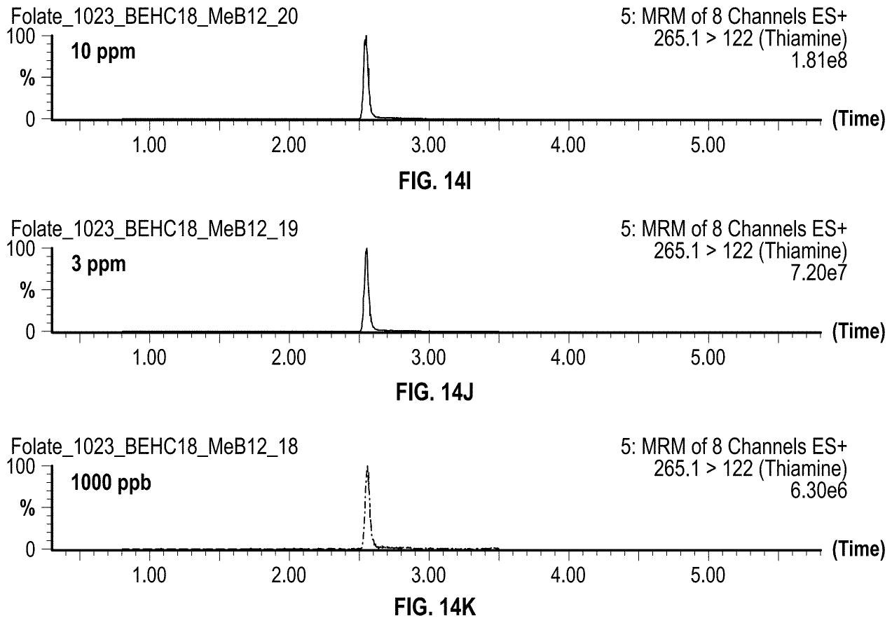

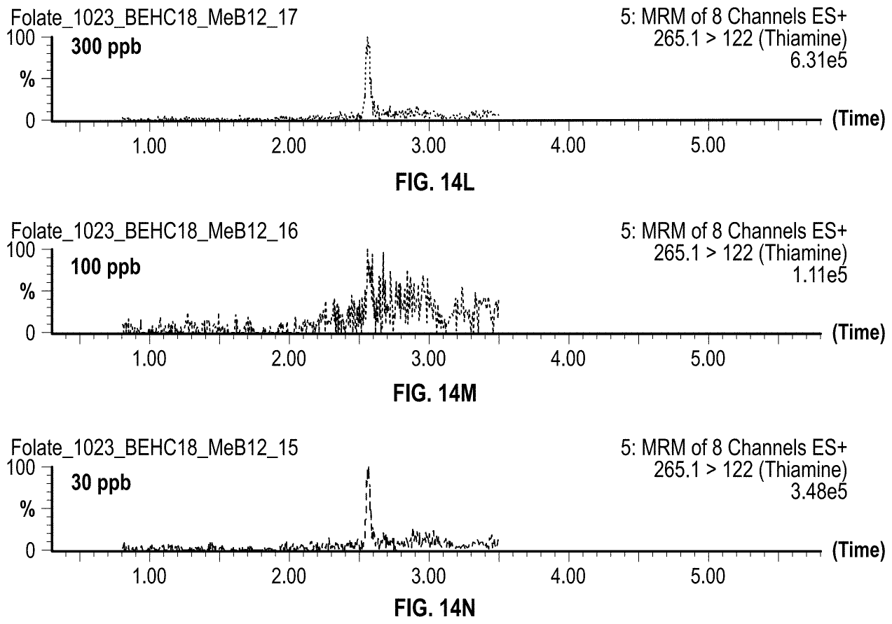

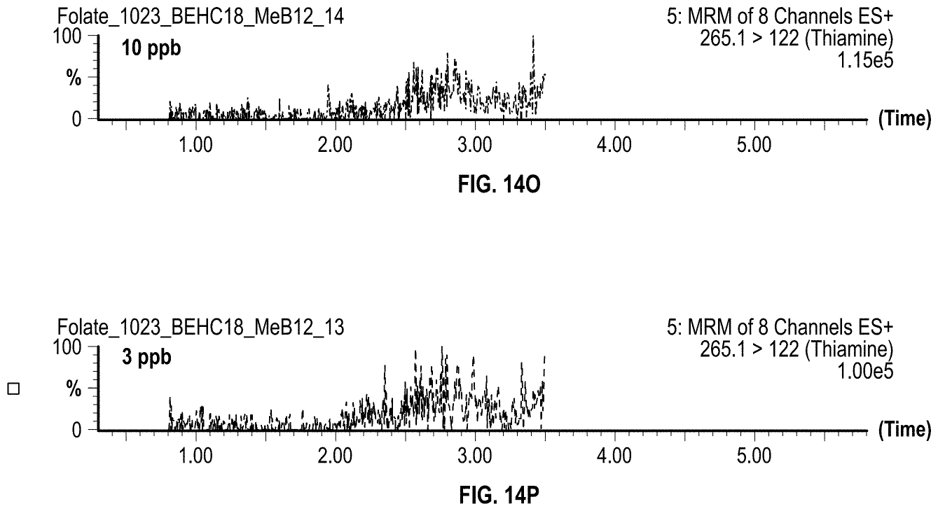

FIGS. 14A-14P compare the chromatograms of thiamine in standard solutions for the SOP configuration (FIGS. 14A-14H) and the LBS configuration (FIG. 14I-FIG. 14P), in accordance with the present disclosure. FIG. 14A, FIG. 14B, FIG. 14C, FIG. 14D, FIG. 14E, FIG. 14F, FIG. 14G and FIG. 14H display chromatograms for 10 ppm, 3 ppm, 1000 ppb, 300 ppb, 100 ppb, 30 ppb, 10 ppb, and 3 ppb of thiamine concentrations, respectively, in standard solutions for the SOP configuration. FIG. 14I, FIG. 14J, FIG. 14K, FIG. 14L, FIG. 14M, FIG. 14N, FIG. 14O and FIG. 14P display chromatograms for 10 ppm, 3 ppm, 1000 ppb, 300 ppb, 100 ppb, 30 ppb, 10 ppb, and 3 ppb of thiamine concentrations, respectively, in standard solutions for the LBS configuration.

The standard concentration ranged from 3 ppb to 10 ppm. For the SOP configuration, as shown in FIGS. 14A-14H, the limit of quantitation (LOQ) was approximately 1000 ppb. For the LBS configuration, as shown in FIGS. 14I-14P, the LOQ was lower at approximately 300 ppb. In addition, peak shape was narrower and showed less tailing for the LBS configuration of FIGS. 14I-14P than the SOP configuration of FIGS. 14A-14H. A comparison of the results of FIGS. 14A-14H to FIGS. 14I-14P, illustrates that using the LBS configuration enhanced sensitivity and peak shape.

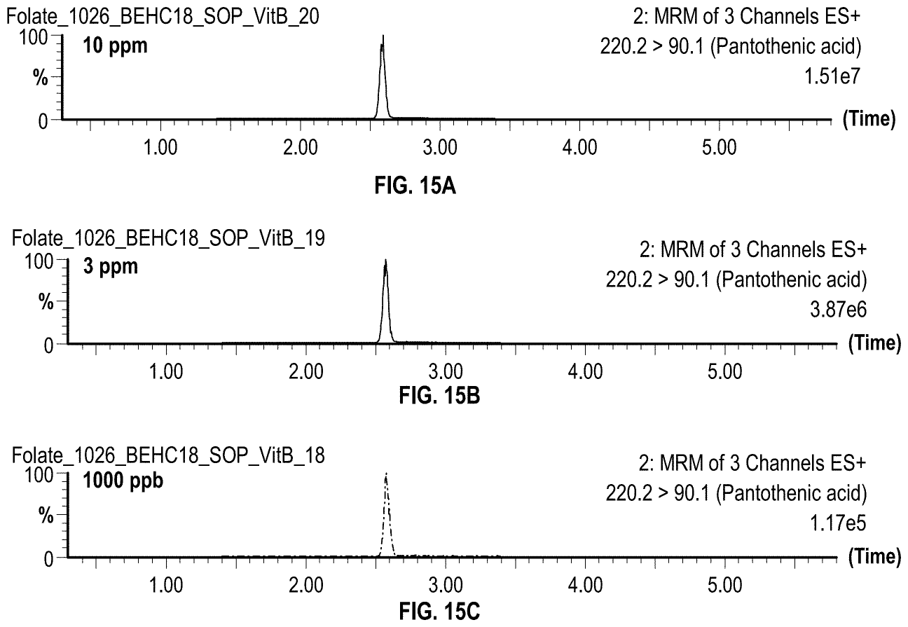

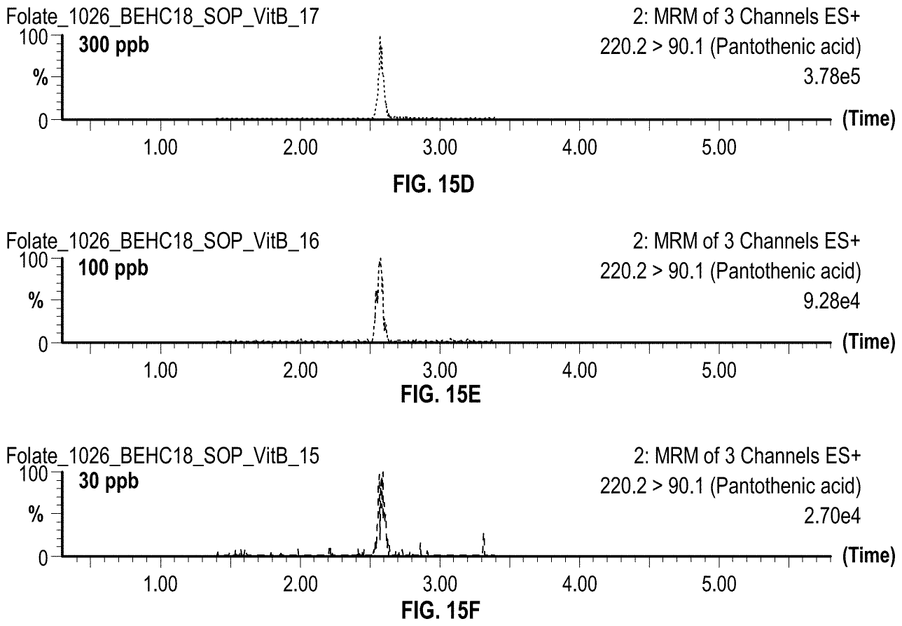

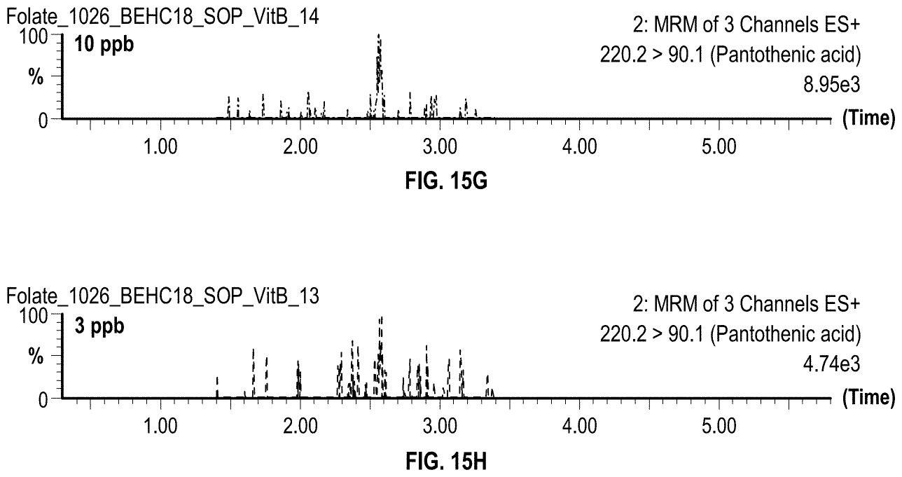

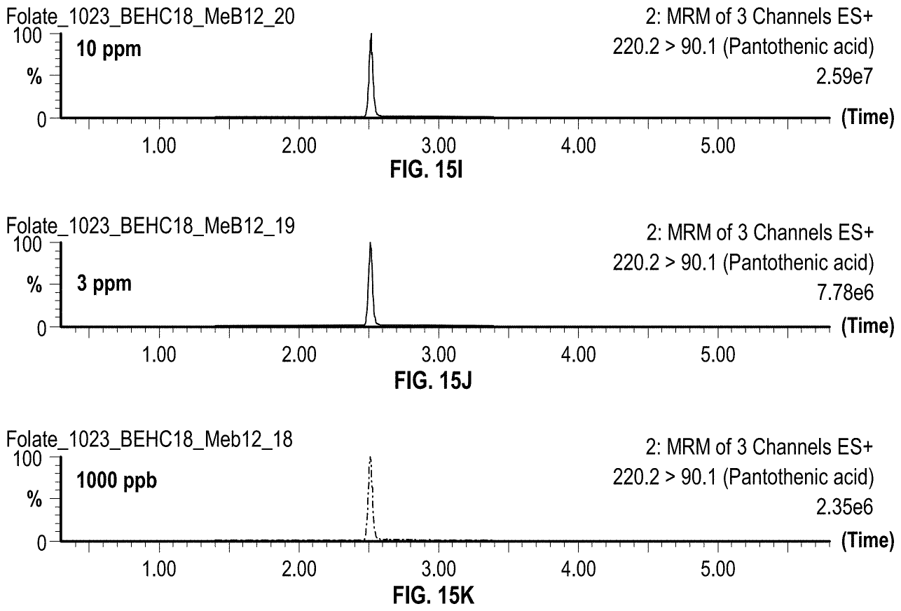

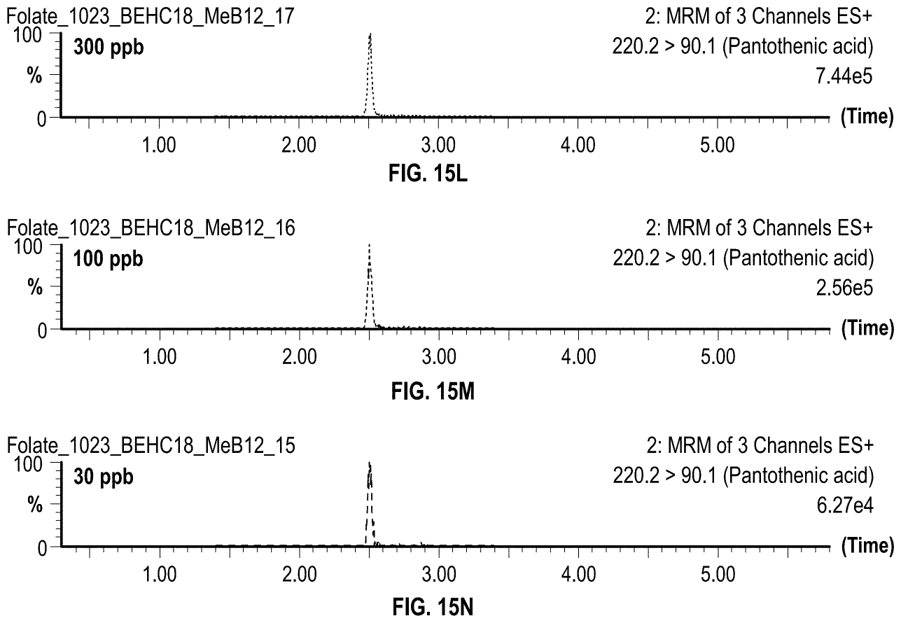

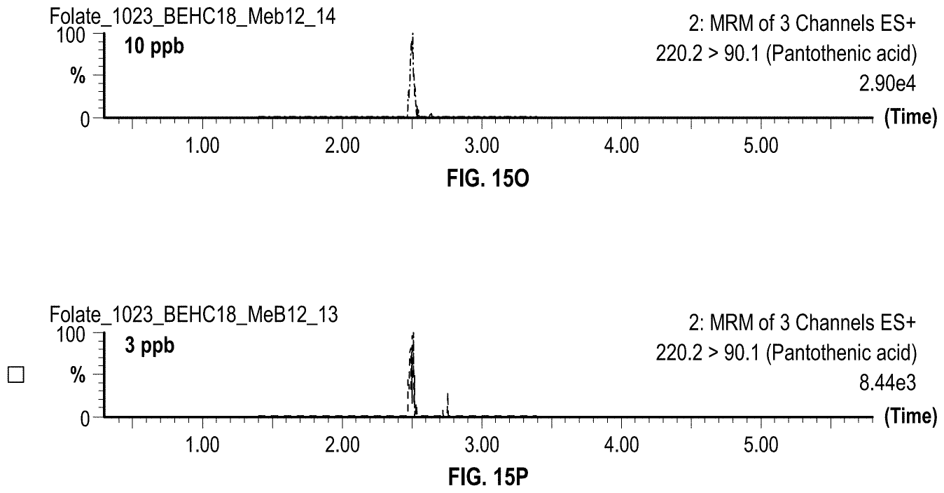

FIGS. 15A-15P compare the chromatograms of pantothenic acid in standard solutions for the SOP configuration (FIGS. 15A-15H) and the LBS configuration (FIGS. 15I-15P), in accordance with the present disclosure. FIG. 15A, FIG. 15B, FIG. 15C, FIG. 15D, FIG. 15E, FIG. 15F, FIG. 15G and FIG. 15H display chromatograms for 10 ppm, 3 ppm, 1000 ppb, 300 ppb, 100 ppb, 30 ppb, 10 ppb, and 3 ppb of pantothenic acid concentrations, respectively, in standard solutions for the SOP configuration. FIG. 15I, FIG. 15J, FIG. 15K, FIG. 15L, FIG. 15M, FIG. 15N, FIG. 15O and FIG. 15P display chromatograms for 10 ppm, 3 ppm, 1000 ppb, 300 ppb, 100 ppb, 30 ppb, 10 ppb, and 3 ppb of pantothenic acid concentrations, respectively, in standard solutions for the LBS configuration.

The standard concentration ranged from 3 ppb to 10 ppm. For the SOP configuration, as shown in FIGS. 15A-15H, the limit of quantitation (LOQ) was approximately 30 ppb. For the LBS configuration, as shown in FIGS. 15I-15P, the LOQ was lower at approximately 10 ppb. In addition, peak shape was slightly narrower for the LBS configuration of FIGS. 15I-15P than the SOP configuration of FIGS. 15A-15H. A comparison of the results of FIGS. 15A-15H to FIGS. 15I-15P, illustrates that using the LBS configuration enhanced sensitivity and peak shape.

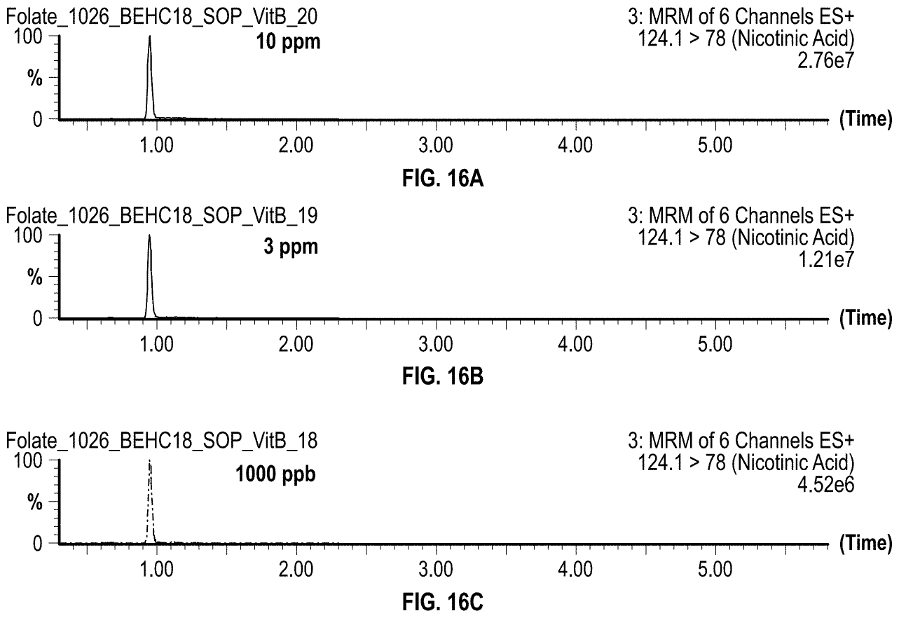

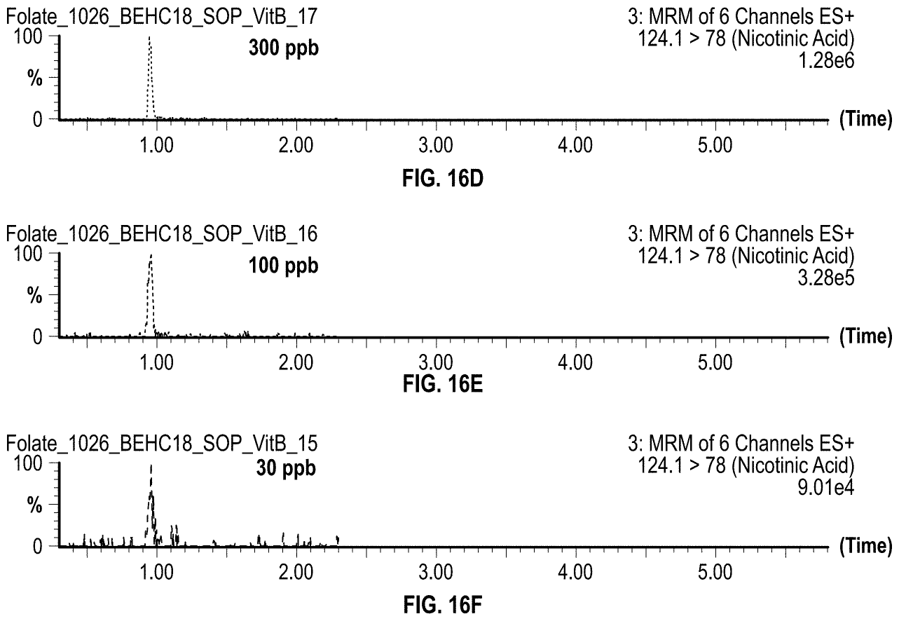

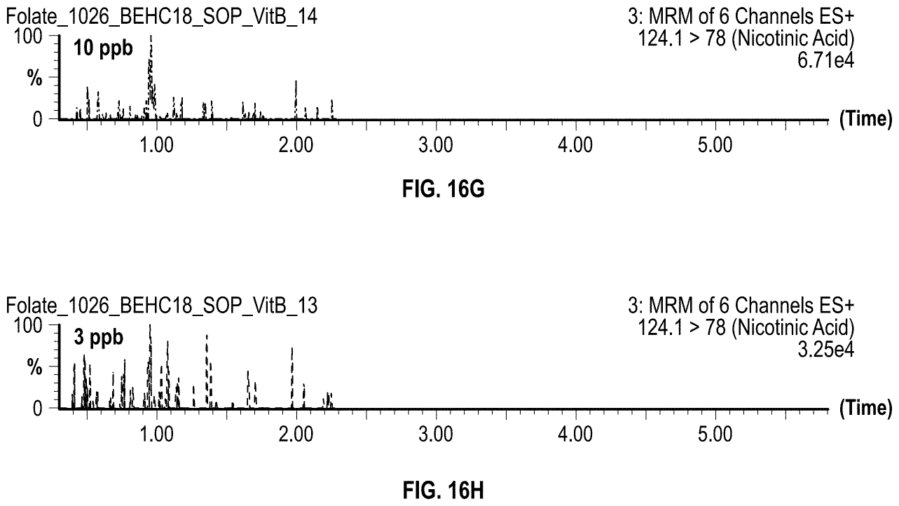

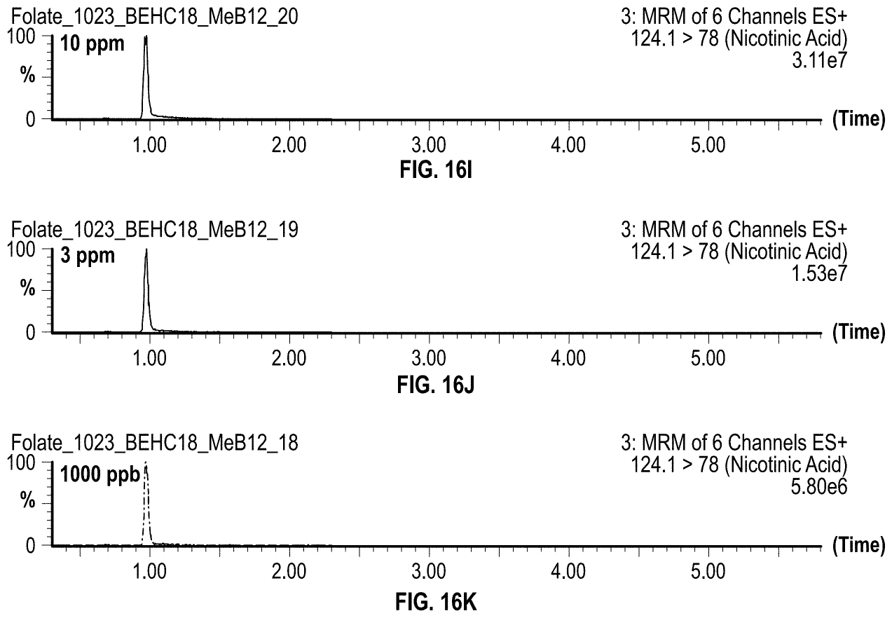

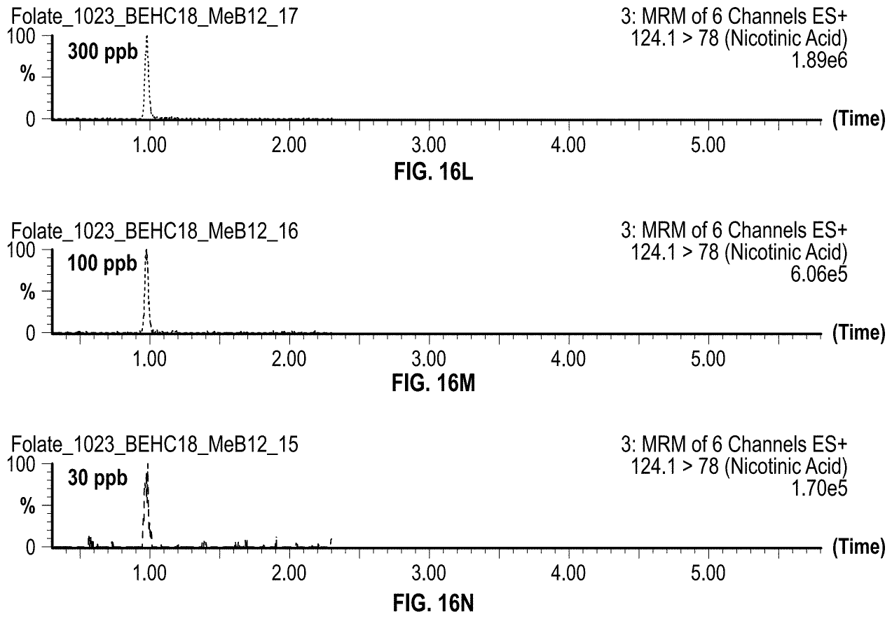

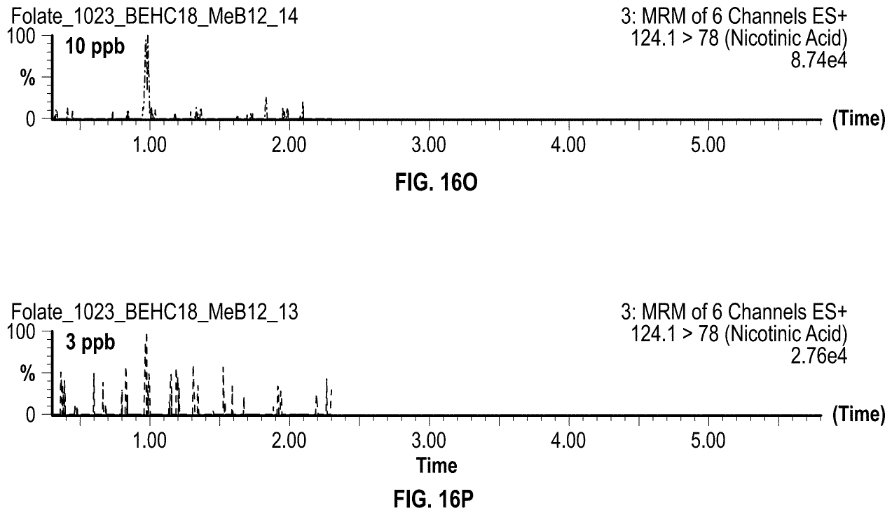

FIGS. 16A-16P compare the chromatograms of nicotinic acid in standard solutions for the SOP configuration (FIGS. 16A-16H) and the LBS configuration (FIGS. 16I-16P), in accordance with the present disclosure. FIG. 16A, FIG. 16B, FIG. 16C, FIG. 16D, FIG. 16E, FIG. 16F, FIG. 16G and FIG. 16H display chromatograms for 10 ppm, 3 ppm, 1000 ppb, 300 ppb, 100 ppb, 30 ppb, 10 ppb, and 3 ppb of nicotinic acid concentrations, respectively, in standard solutions for the SOP configuration. FIG. 16I, FIG. 16J, FIG. 16K, FIG. 16L, FIG. 16M, FIG. 16N, FIG. 16O and FIG. 16P display chromatograms for 10 ppm, 3 ppm, 1000 ppb, 300 ppb, 100 ppb, 30 ppb, 10 ppb, and 3 ppb of nicotinic acid concentrations, respectively, in standard solutions for the LBS configuration.

The standard concentration ranged from 3 ppb to 10 ppm. For the SOP configuration, as shown in FIGS. 16A-16H, the limit of quantitation (LOQ) was approximately 50 ppb. For the LBS configuration, as shown in FIGS. 16I-16P, the LOQ was lower at approximately 30 ppb. A comparison of the results of FIGS. 16A-16H to FIGS. 16I-16P, illustrates that using the LBS configuration enhanced sensitivity.

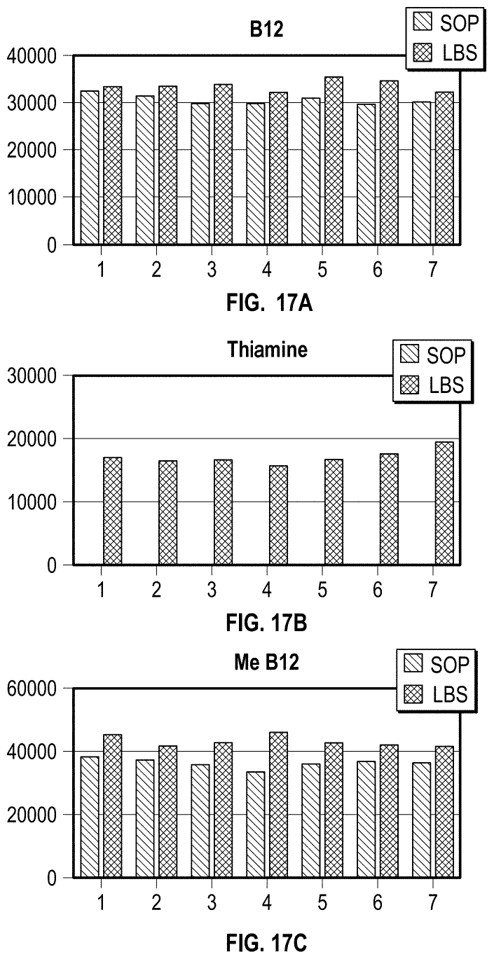

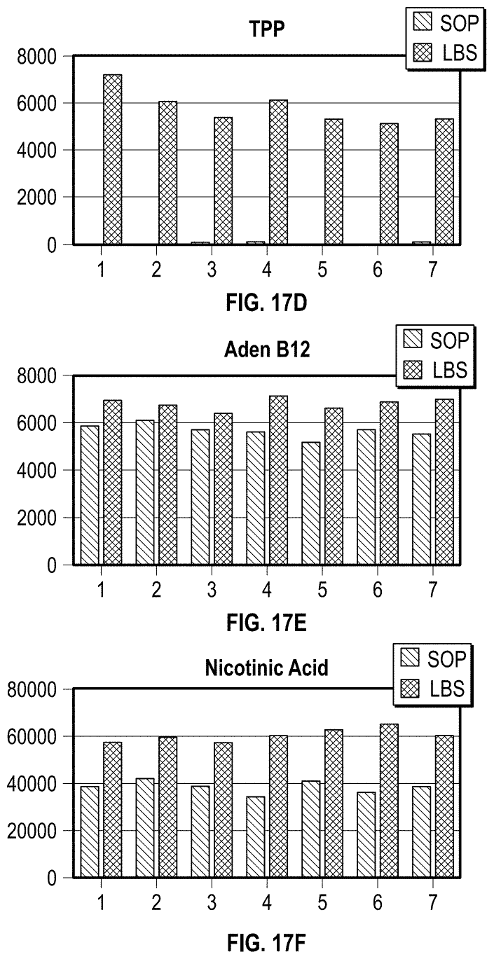

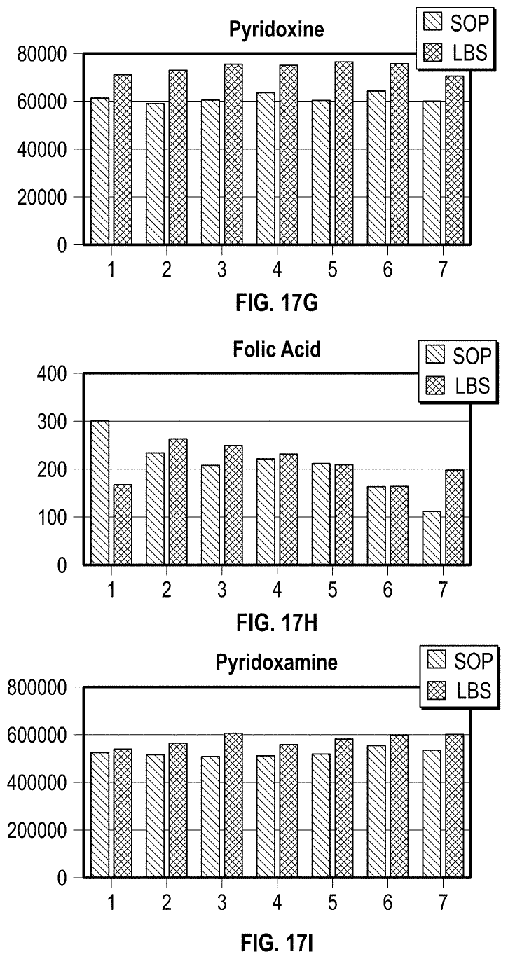

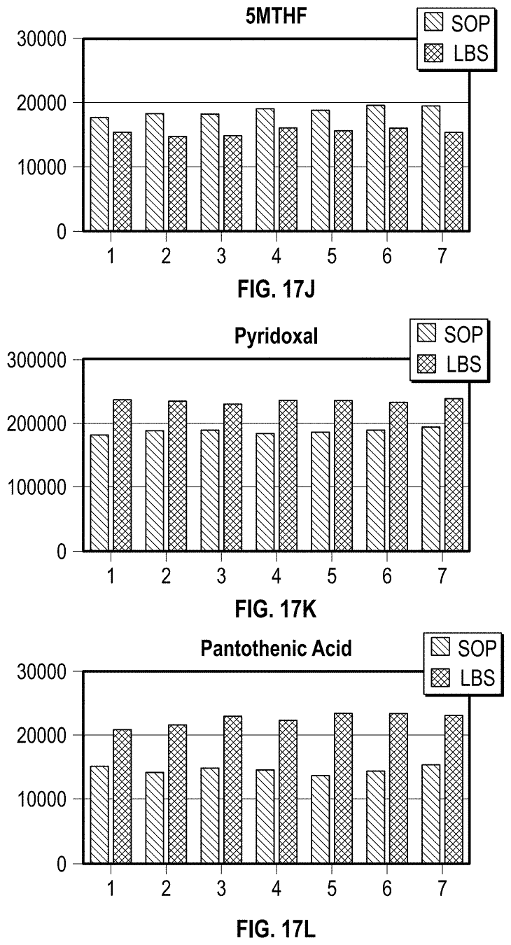

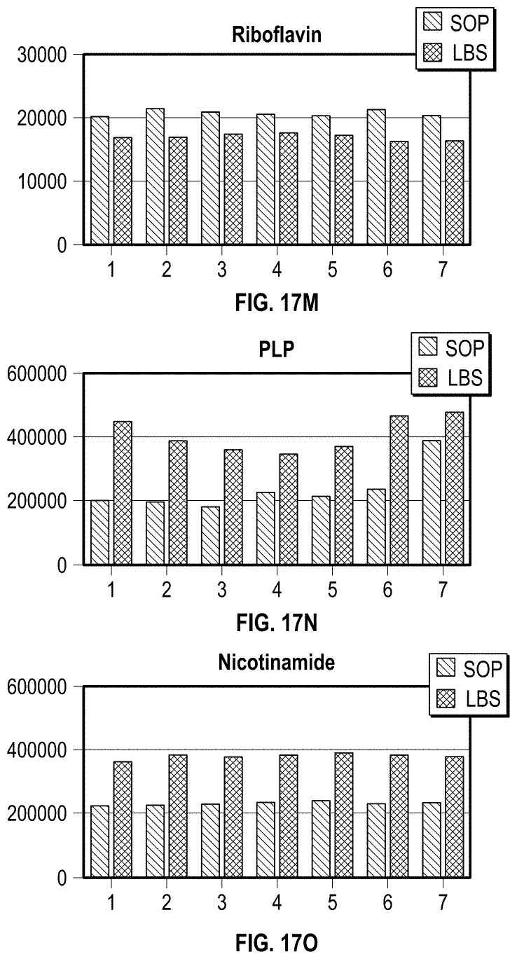

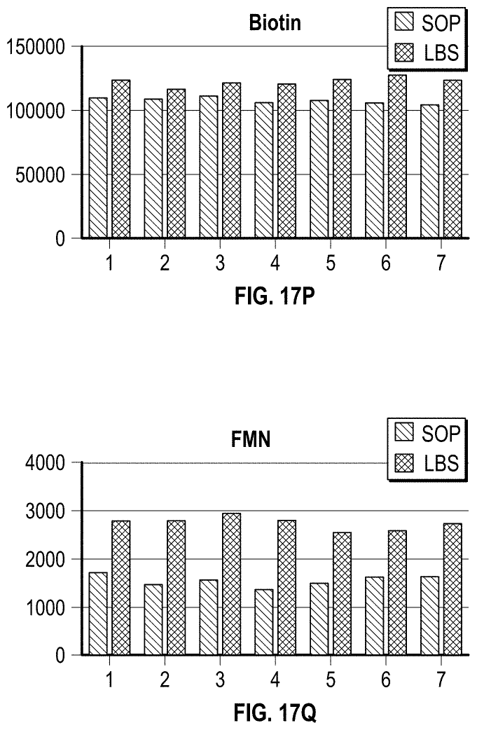

FIGS. 17A-17Q, compare peak areas for the SOP configuration and LBS configuration after initial injection when the systems and columns had been used lightly. Peak areas from standard surface (SOP configuration) are plotted side by side with those from LBS surface (LBS configuration). FIG. 17A, FIG. 17B, FIG. 17C, FIG. 17D, FIG. 17E, FIG. 17F, FIG. 17G, FIG. 17H, FIG. 17I, FIG. 17J, FIG. 17K, FIG. 17L, FIG. 17M, FIG. 17N, FIG. 17O, FIG. 17P and FIG. 17Q are similar comparison to the comparison of FIG. 8A-FIG. 8R but repeated after the initial injections. For FIG. 17A-FIG. 17Q, the comparison of the configurations is completed after 31 injections of standard solutions at various concentrations ranging from 3 ppb to 10 ppm. The comparison was conducted with a standard mix solution at 0.3 ppm, or 0.3 μg/ml. The columns and systems of FIG. 17A-FIG. 17Q were conditioned with blank injections, and no cleaning or flushing was done. Observations for FIG. 17A-FIG. 17Q were similar to the observations of FIG. 8A-FIG. 8R. Large differences for TPP, Thiamine, FMN (flavin mononucleotide), PLP, and pantothenic acid were observed. The peak areas of these compounds from the LBS configuration were significantly larger than those peak areas from the SOP configuration. The TPP and thiamine analysis did not show any peak in the SOP configuration under these conditions. Other compounds, such as cyanocobalamine (B12), methyl-cobalamine (MeB12), adenosyl-cobalamin (Aden B12), nicotinic acid, nicotinamide, pyridoxine, pyridoxaminde, pyridoxal, and biotin show larger peak areas for the LBS configuration than those in the SOP configuration.

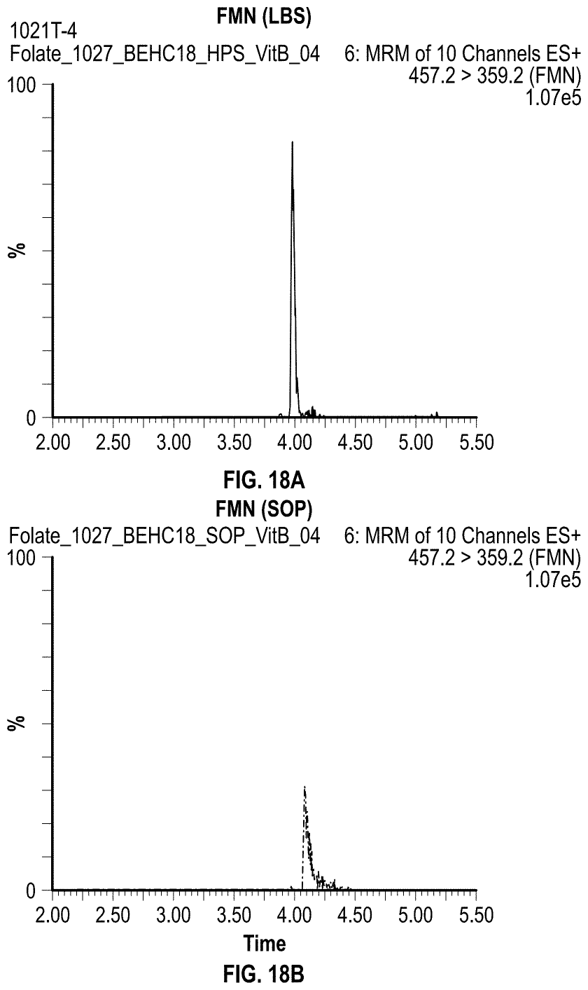

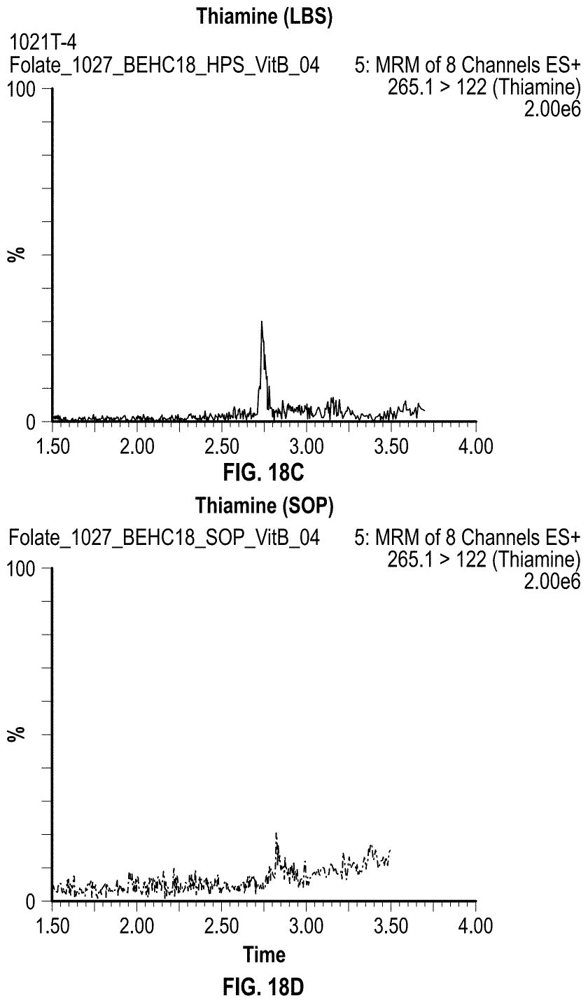

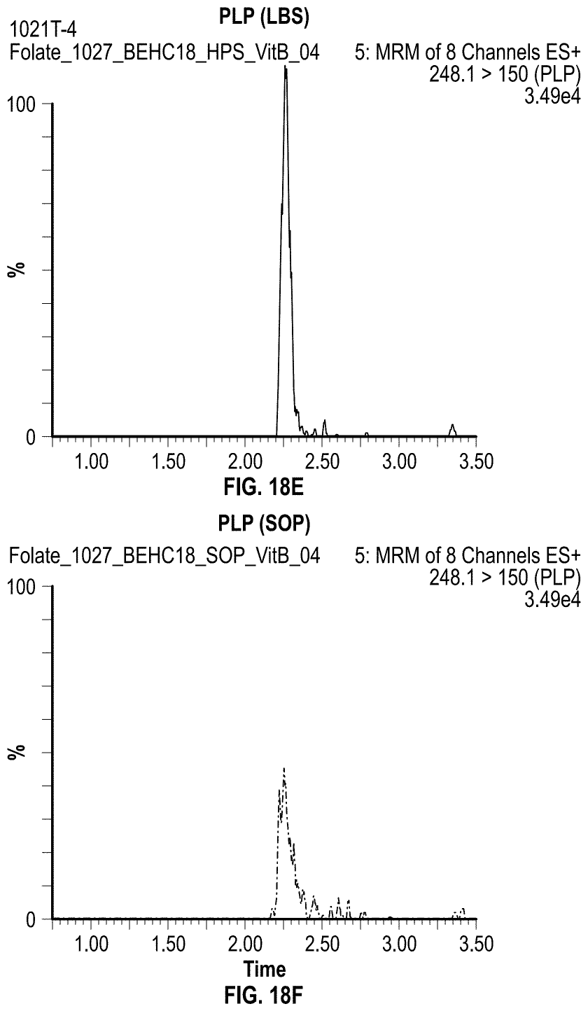

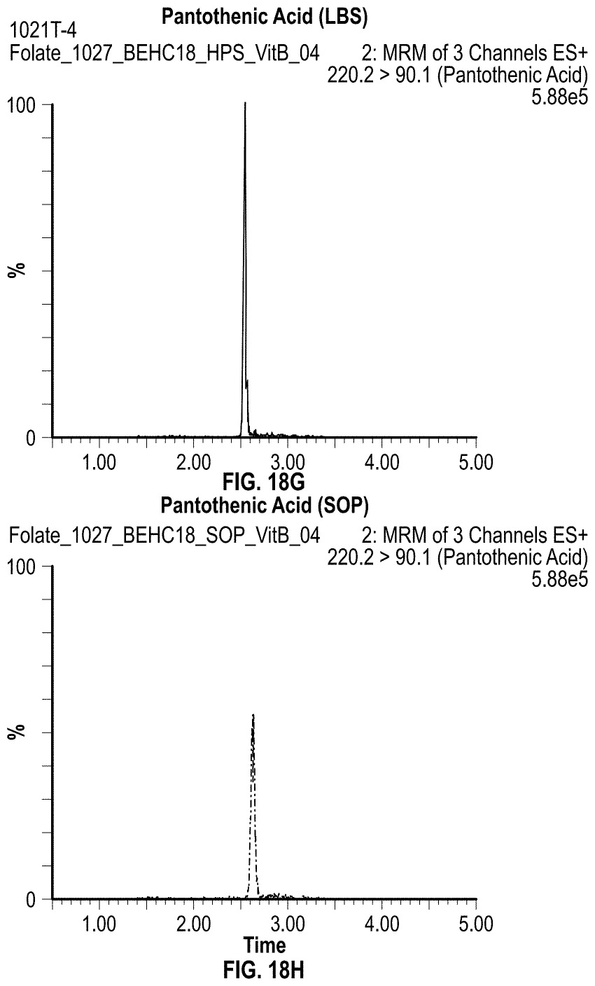

FIG. 18A, FIG. 18B, FIG. 18C, FIG. 18D, FIG. 18E, FIG. 18F, FIG. 18G, FIG. 18H, FIG. 18I and FIG. 18J compare the chromatograms for the LBS configuration and the SOP configuration for flavin mononucleotide (FMN), thiamine (B₁), pyridoxal 5′-phosphate (PLP), pantothenic acid (B₅), and thiamine pyrophosphate (TPP) respectively, in accordance with the present disclosure. FIG. 18A, FIG. 18C, FIG. 18E, FIG. 18G and FIG. 18I display the chromatograms for the LBS configuration for flavin mononucleotide (FMN), thiamine (B₁), pyridoxal 5′-phosphate (PLP), pantothenic acid (B₅), and thiamine pyrophosphate (TPP) respectively, in accordance with the present disclosure. FIG. 18B, FIG. 18D, FIG. 18F, FIG. 18H and FIG. 18J display the chromatograms for the SOP configuration for flavin mononucleotide (FMN), thiamine (B₁), pyridoxal 5′-phosphate (PLP), pantothenic acid (B₅), and thiamine pyrophosphate (TPP) respectively, in accordance with the present disclosure.

Vertical axes are the same scales. The standard concentration was 0.3 ppm (μg/ml). The chromatograms of FIG. 18A, FIG. 18C, FIG. 18E, FIG. 18G and FIG. 18I show the positive effect the LBS configuration has on peak area (peak intensity) after initial injections. FIG. 18A-FIG. 18J provide a similar comparison to the comparison of FIG. 9A-FIG. 9H but repeated after the initial injections. For FIG. 18A-FIG. 18J, the initial injections were 31 injections of standard solutions at various concentrations ranging from 3 ppb to 10 ppm.

To summarize the results for sensitivity, the use of LBS in LC system and column helps to increase the peak area (intensity), reduce peak tailing and peak width for B vitamins, which result in better detection sensitivity. The peak areas of thiamine, TPP, FMN, PLP, pantothenic acid were significantly increased in the LBS configuration than those obtained in the SOP configuration at concentration 1 ppm (1 μg/ml) during the initial injections of B vitamin standard mix solutions onto a new column in a flush and cleaned LC system. Marginal increase in peak area was observed for cyanocobalamin, methyl-cobalamin, adenosyl-cobalamin, nicotinic acid, nicotinamide, pyridoxine, pyridoamine, pyridoxal, biotin. No difference in peak area was observed for riboflavin, NAD, folic acid, and 5-Methyl-THF. A second comparison (FIG. 17A-FIG. 17Q and FIG. 18A-FIG. 18J) of chromatograms of a standard mix solution at 0.3 ppm of each standard from both SOP and LBS configurations showed a similar observation. The second comparison was conducted when the system and column had been lightly used (31 injections of standard solutions at various concentration). As a result, higher detection sensitivities for some B vitamins were obtained in the LBS configuration. No significant detrimental effect was observed for B vitamins in the LBS configuration.

The increased responses of the LBS configuration lead to higher sensitivities in the LC-MS analysis of the B vitamins. The limit of quantification (LOQ) in both the SOP configuration and LBS configuration was estimated using the same standard solutions at concentrations ranging from 3 ng/mL to 10 μg/mL. Large improvement in LOQ (3-10 times improvement) was achieved for FMN, thiamine, PLP and pantothenic acid in the LBS system setup (Table 1).

TABLE 1
Estimated LOQ values for selected B vitamins

| | | | LOQ |
|---|---|---|---|
| | LOQ (ng/mL) | | improvement |
| | SOP | LBS | in LBS than |
| Compound | configuration | configuration | SOP setup |
| FMN | 1000 | 100 | 10 times |
| Thiamine | 1000 | 300 | 3 times |
| PLP | 1000 | 300 | 3 times |
| Pantothenic acid | 30 | 10 | 3 times |

Regarding carry-over, the LBS configuration minimizes interactions between the B vitamins and the stainless-steel inner surface of the flow path in LC system and column, and reduces the risk of carry-over that often occurs in the LC analysis of B-group of vitamins.

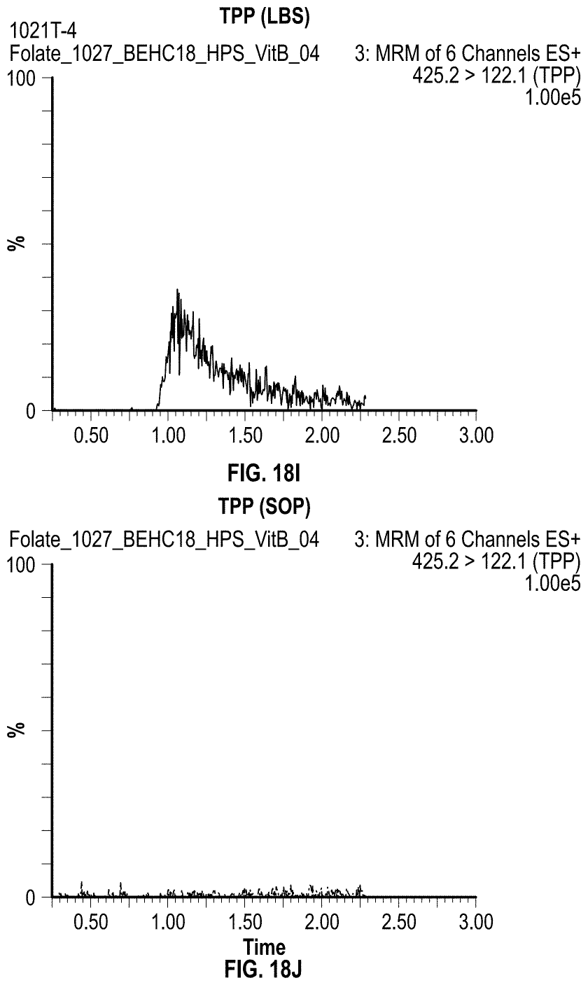

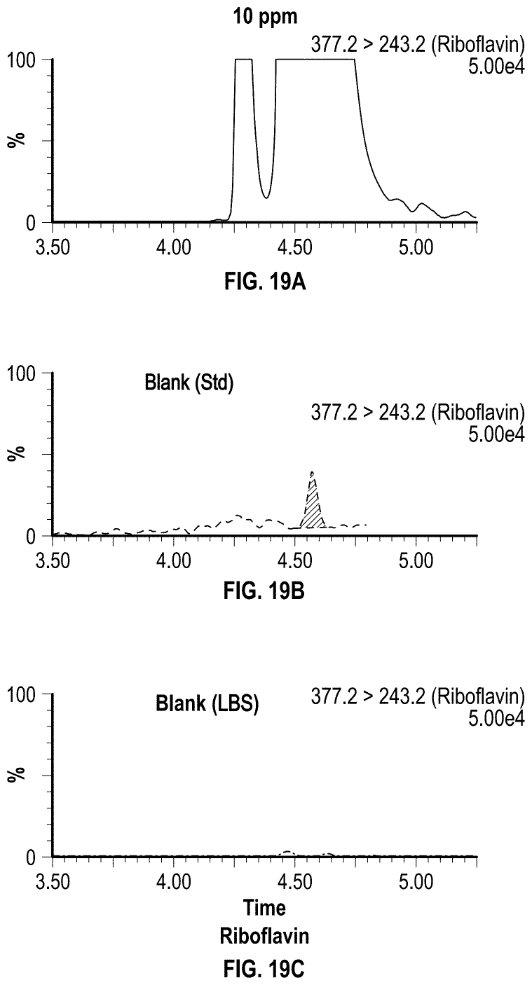

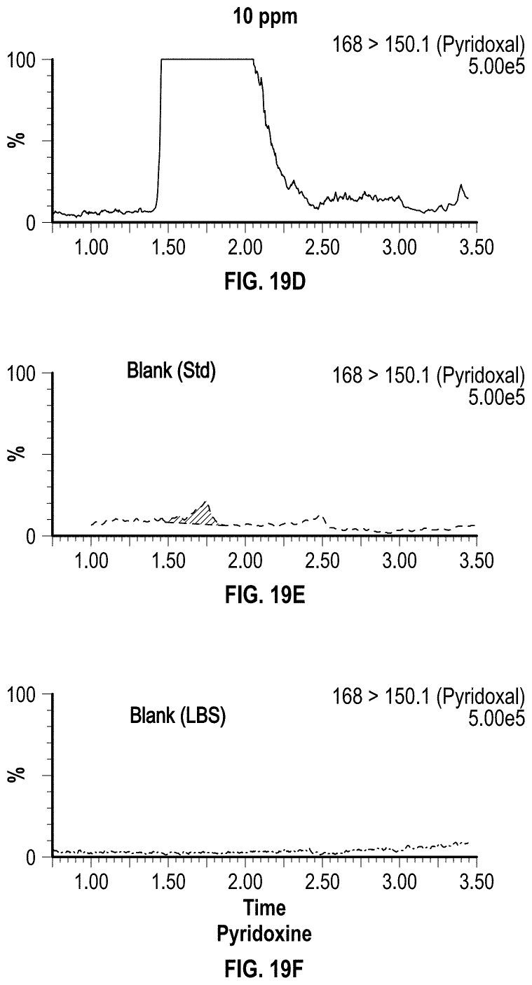

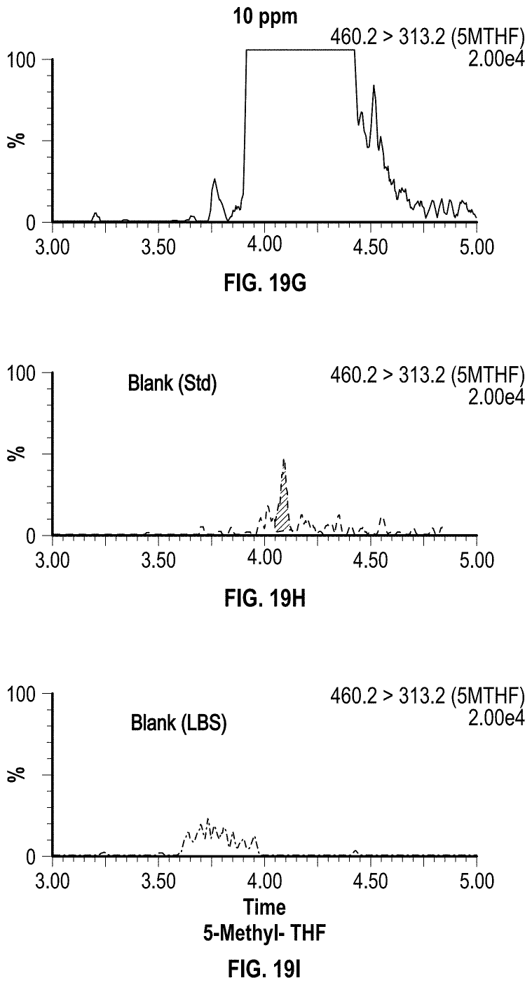

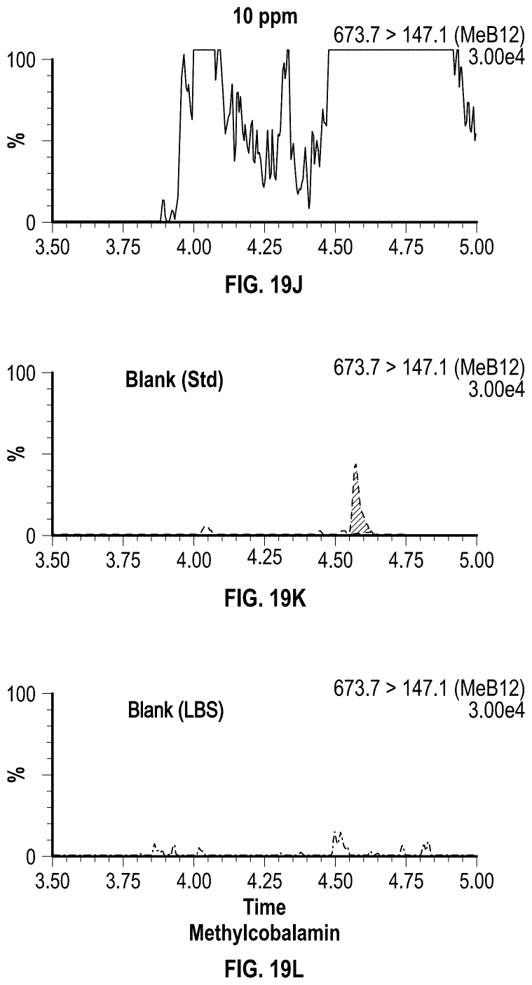

FIG. 19A, FIG. 19B, FIG. 19C, FIG. 19D, FIG. 19E, FIG. 19F, FIG. 19G, FIG. 19H, FIG. 19I, FIG. 19J, FIG. 19K and FIG. 19L show a comparison of the selected B vitamins LC-MS chromatograms obtained with the LBS and the SOP configurations in a carry-over study. FIG. 19A, FIG. 19D, FIG. 19G and FIG. 19J display LC-MS chromatograms of injections of a 10 ppm standard solution for riboflavin, pyridoxal, 5-methyl-THF, and methylcobalamin (Me B12), respectively, in accordance with the present disclosure. FIG. 19B, FIG. 19E, FIG. 19H and FIG. 19K display LC-MS chromatograms of blank injections obtained with the SOP configurations for riboflavin, pyridoxal, 5-methyl-THF, and methylcobalamin (Me B12), respectively, in accordance with the present disclosure. FIG. 19C, FIG. 19F, FIG. 19I and FIG. 19L display LC-MS chromatograms of blank injections obtained with the LBS configurations for riboflavin, pyridoxal, 5-methyl-THF, and methylcobalamin (Me B12), respectively, in accordance with the present disclosure.

For FIG. 19A-FIG. 19L, carry-over was not observed using the LBS configuration. Comparison of LC-MS chromatograms of blank injections obtained with the LBS configuration and with SOP configuration right after the injections of a 10 ppm standard solution for riboflavin (FIGS. 19A-19C), pyridoxine (FIGS. 19D-19F), 5-methyl-THF (FIGS. 19G-19I), and methylcobalamin (FIGS. 19J-19L). Residual peaks were found in the blank injection on the SOP configuration for riboflavin, pyridoxine, 5-methyl-tetrahydrofolate, and methylcobalamin while no residual peak was found on the LBS configuration. The LBS configuration reduces the risk of carry-over in the LC-MS analysis for B vitamins.

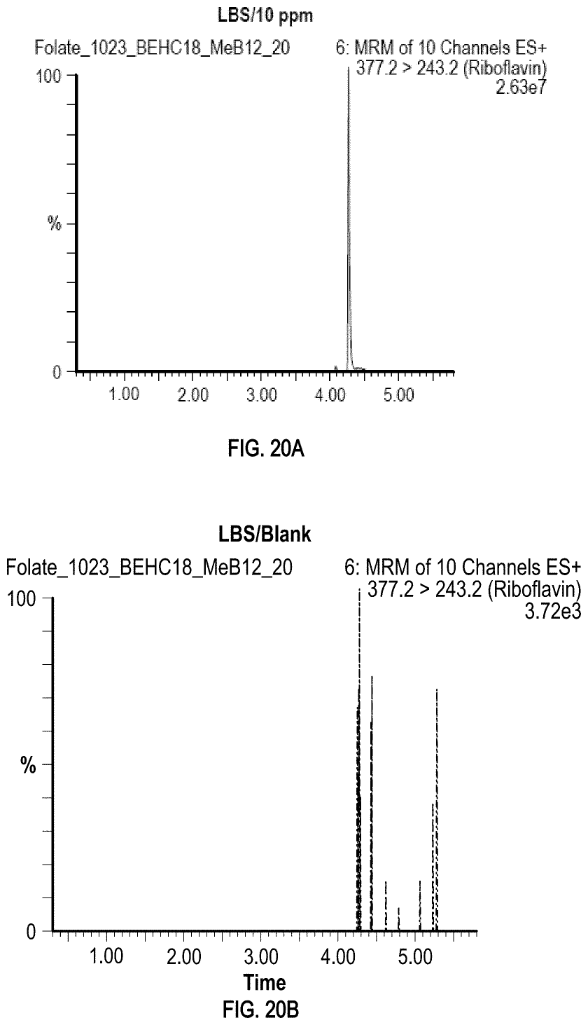

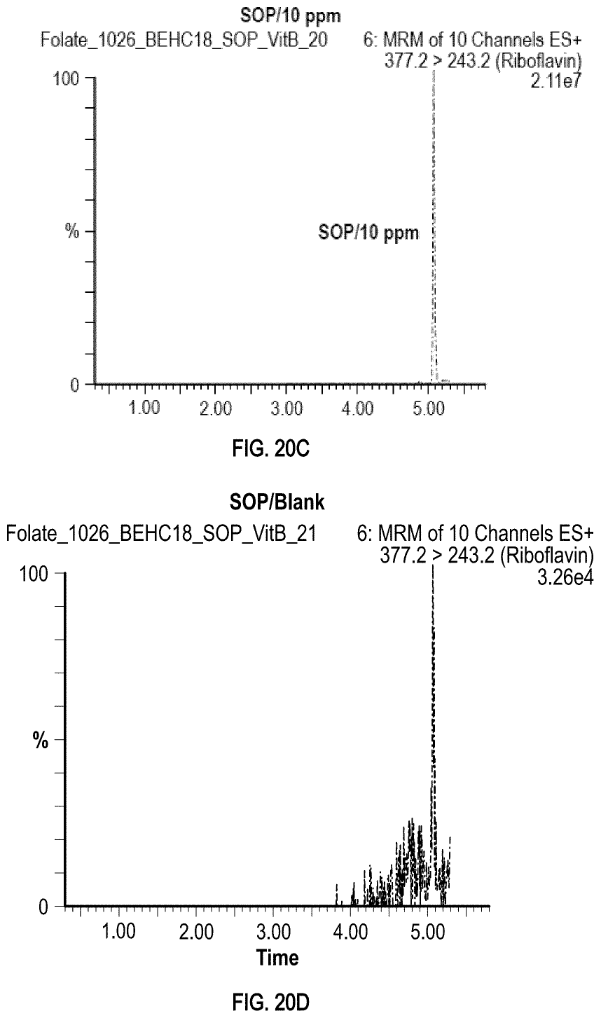

FIG. 20A, FIG. 20B, FIG. 20C and FIG. 20D display the chromatograms for the LBS configuration and the SOP configuration for selected B vitamins to compare carry-over for riboflavin. FIG. 20B shows a blank injection right after an injection of a high concentration standard solution (10 ppm) (FIG. 20A) for a LBS configuration. FIG. 20D shows a blank injection (FIG. 20D) right after an injection of the high concentration standard solution (10 ppm) (FIG. 20C) for a SOP configuration. For riboflavin, the blank injection in the SOP configuration showed a residue peak at about 0.1% of previous peak's area, and the blank injection showed no residue peak in the LBS configuration.

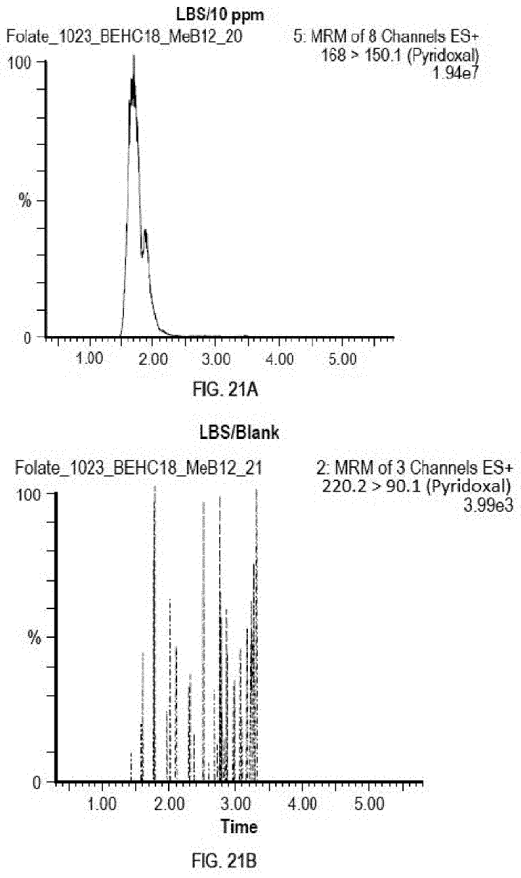

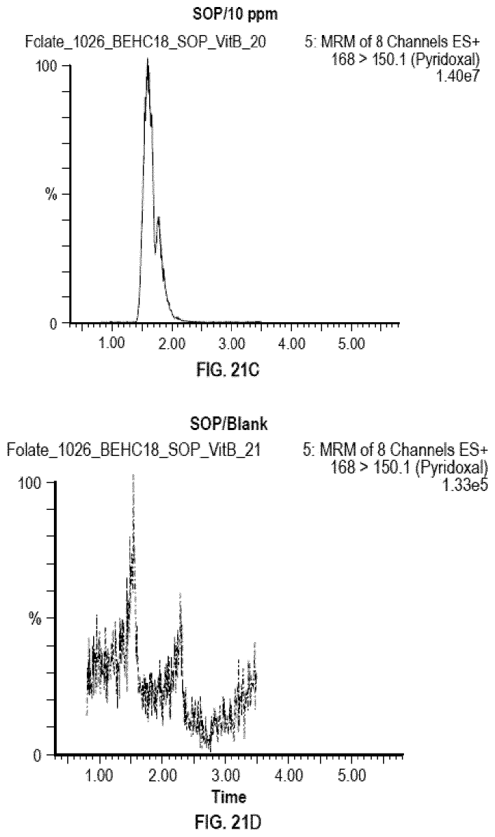

FIG. 21A, FIG. 21B, FIG. 21C and FIG. 21D display the chromatograms for the LBS configuration and the SOP configuration for selected B vitamins to compare carry-over for pyridoxal. FIG. 21B shows a blank injection right after an injection of a high concentration standard solution (10 ppm) (FIG. 21A) for a LBS configuration. FIG. 21D shows a blank injection right after an injection of the high concentration standard solution (10 ppm) (FIG. 21C) for the SOP configuration. For pyridoxal, the blank injection in the SOP configuration showed a residue peak at about 0.1% of previous peak's area, and the blank injection showed no residue peak in the LBS configuration.

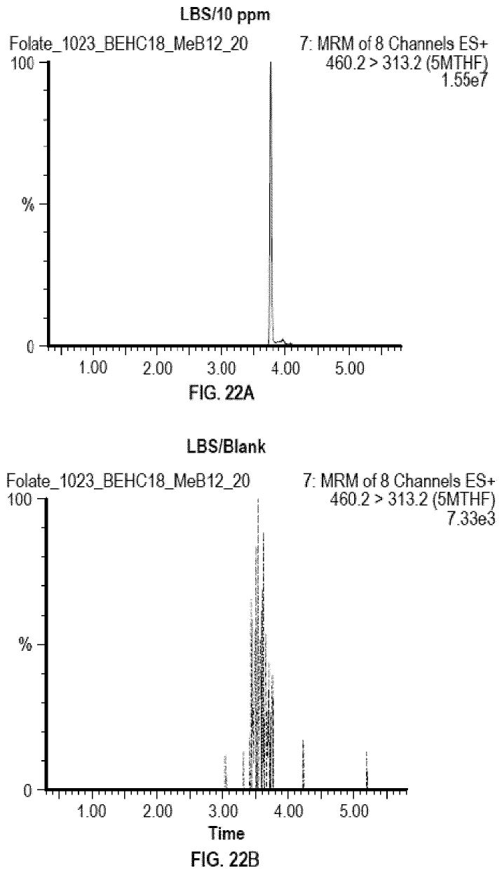

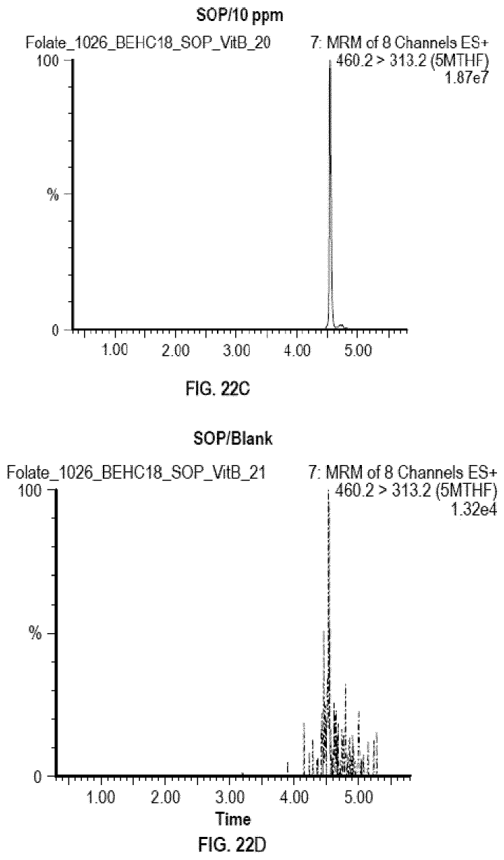

FIG. 22A, FIG. 22B, FIG. 22C and FIG. 22D display the chromatograms for the LBS configuration and the SOP configuration for selected B vitamins to compare carry-over for 5-methyl-THF. FIG. 22B shows a blank injection right after an injection of a high concentration standard solution (10 ppm) (FIG. 22A) for the LBS configuration. FIG. 22D shows a blank injection right after an injection of the high concentration standard solution (10 ppm) (FIG. 22C) for the SOP configuration. For 5-methyl-THF, the blank injection in the SOP configuration showed a residue peak at about 0.1% of previous peak's area, and the blank injection showed no residue peak in the LBS configuration.

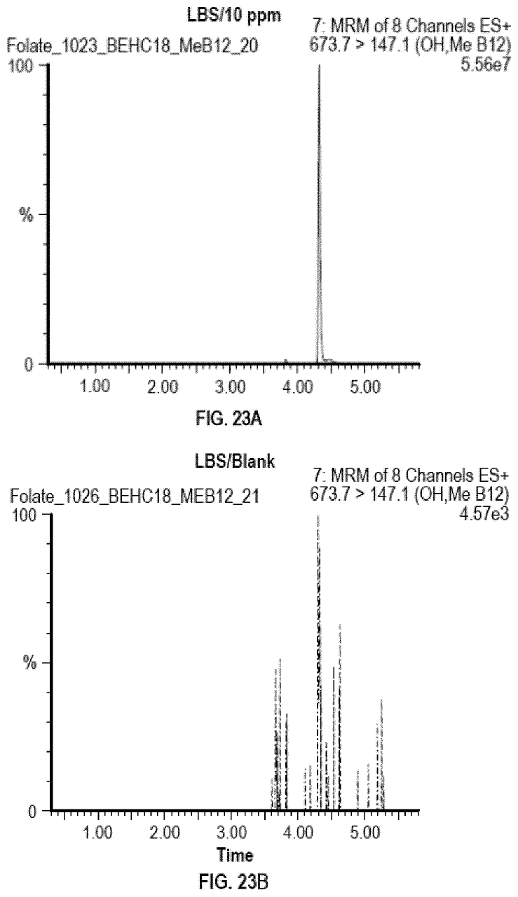

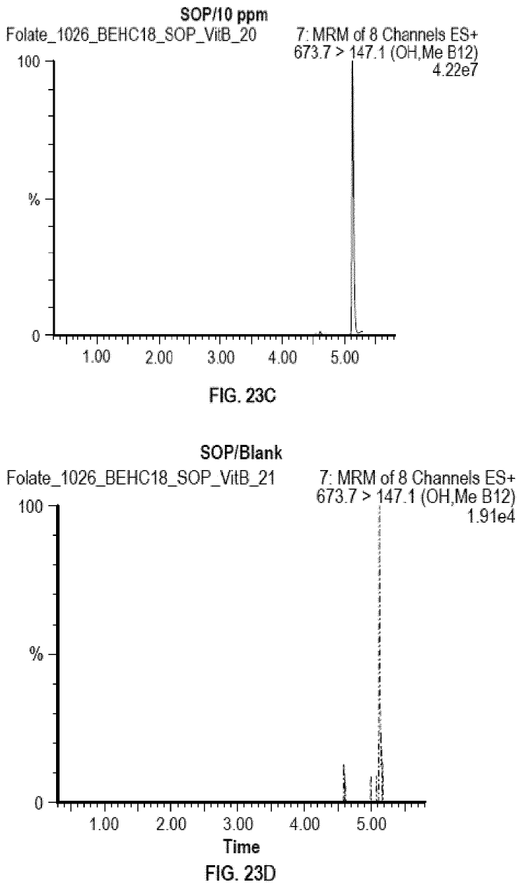

FIG. 23A, FIG. 23B, FIG. 23C and FIG. 23D display the chromatograms for the LBS configuration and the SOP configuration for selected B vitamins to compare carry-over for methyl-cobalamin. FIG. 23B shows a blank injection right after an injection of a high concentration standard solution (10 ppm) (FIG. 23A) for the LBS configuration. FIG. 23D show a blank injection right after an injection of the high concentration standard solution (10 ppm) (FIG. 23C) for the SOP configuration. For methyl-cobalamin, the blank injection in the SOP configuration showed a residue peak at about 0.03% of previous peak's area, and the blank injection showed no residue peak in the LBS configuration.

No significant carry-over issue exists for B vitamins in both the LBS configuration and the SOP configuration. Small residue peaks for riboflavin, pyridoxal, 5-methyl-THF and methyl-cobalamin were observed in blank injections following an injection of a high concentration standard mix solution (10 ppm) for the SOP configuration. The residue peaks were about 0.1% or less of the previous 10 ppm standard peak areas. In contrast, for the LBS configuration, there was no residue peak found in the blank injection for B vitamins. The use of LBS in LC systems and columns helps to reduce the potential carry-over issue for B vitamins.

The effect of LBS on the analysis of B vitamins includes the response factors, which were calculated using the calibration data in concentration range close to their LOQ. The response factors are more comprehensive than a single concentration level comparison in term of comparing the response in two configurations for the LBS configuration and the SOP configuration. That is, comparison at a fix concentration level may not reflect the complete picture since the LOQ and the linearity for the B vitamins are not the same. Some B vitamins are best fitted with quadratic curves. If the concentration level is too low or too high, the difference observed at that concentration would appear larger or smaller. Response factors for B vitamins on the LBS configuration and the SOP configuration (the same Xevo TQ-S Micro System coupled to two LC systems) were compared during extensive use (up to 170 injections).

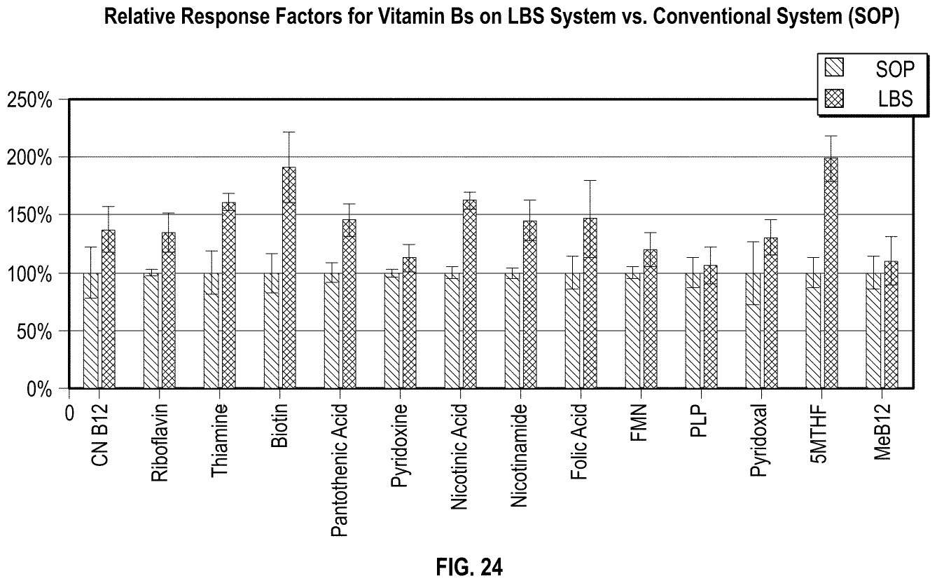

FIG. 24 displays the relative response factors for B vitamins on the LC-MS systems comparing the LBS configuration to the SOP configuration during the LC-MS analysis of B vitamins. Calibration standard solutions that were injected during the B vitamin analysis were used for the response factors, which were calculated based on the calibration data points in low concentration ranges above their LOQs. Data was collected during an extensive use of the system, in which total of 170 injections of standard solutions, sample solutions, and blanks were made on both configurations (LBS and SOP). The response factors were calculated from the calibration data points obtained at different periods. To obtain relative response factors, the response factors from the LBS configuration were normalized to those from the SOP configuration for each compound. The average values were plotted with the error bar represents the ±RSD in the response factor (n=4). After extensive use, the LBS configuration still exhibits higher response factors (>150%) for some B vitamins: such as thiamine, biotin, nicotinic acid, and 5MTHF.

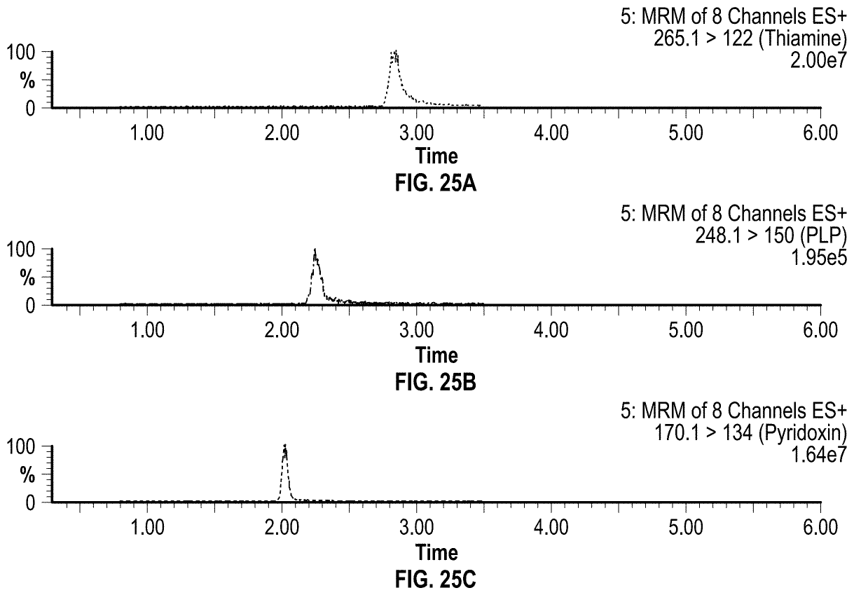

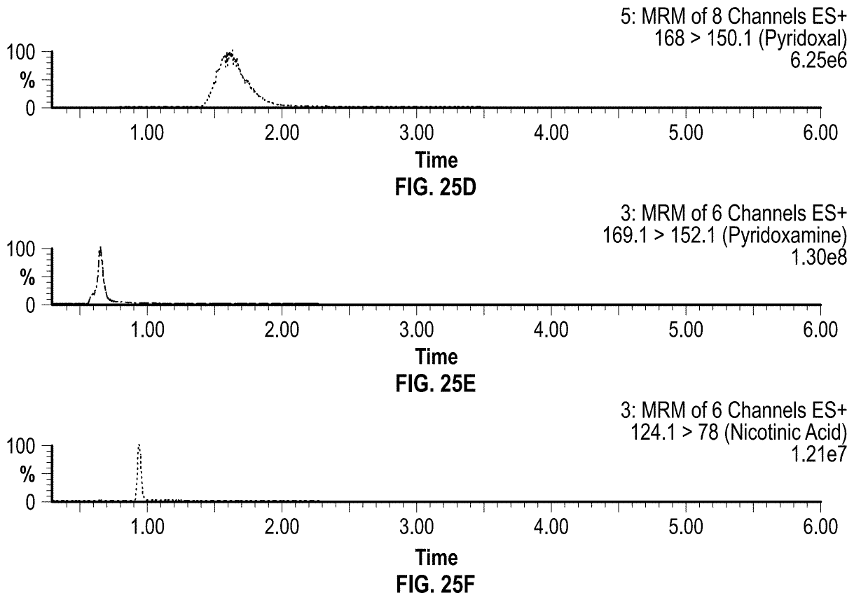

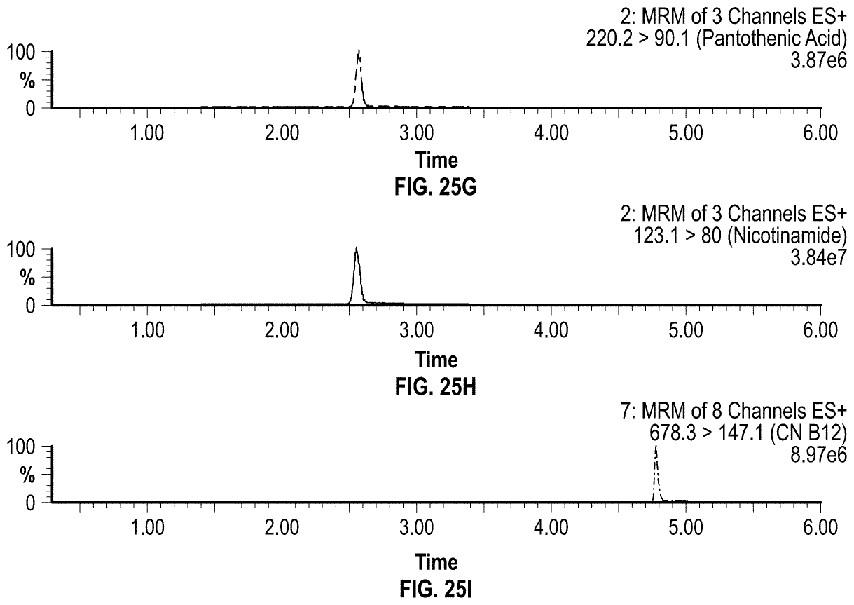

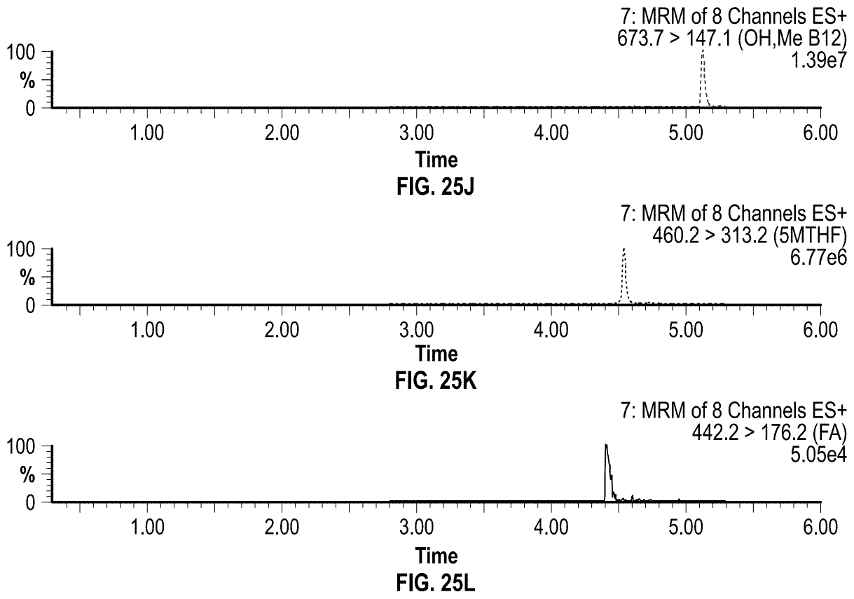

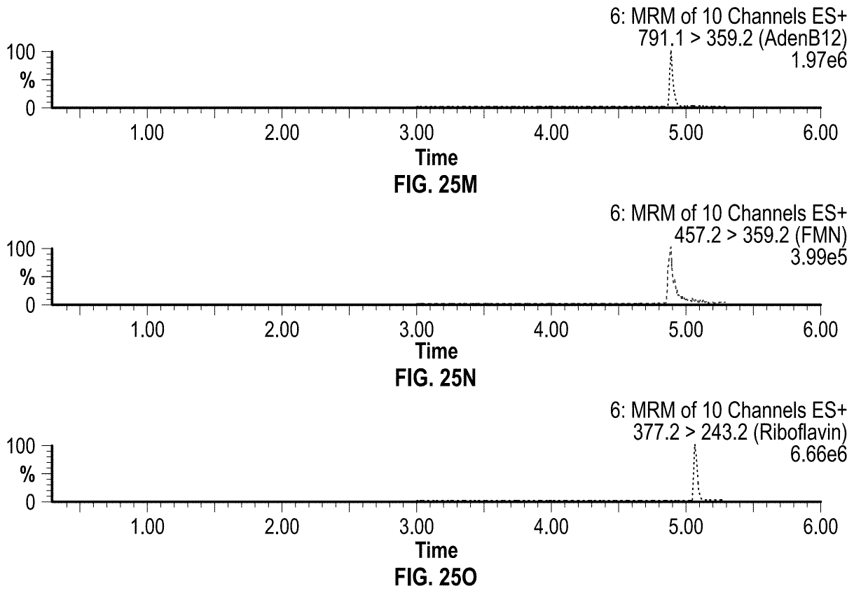

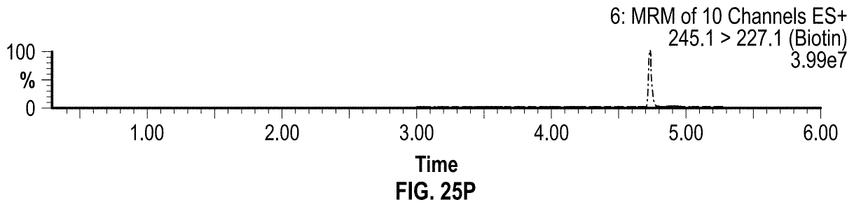

FIG. 25A, FIG. 25B, FIG. 25C, FIG. 25D, FIG. 25E, FIG. 25F, FIG. 25G, FIG. 25H, FIG. 25I, FIG. 25J, FIG. 25K, FIG. 25L, FIG. 25M, FIG. 25N, FIG. 25O and FIG. 25P displays typical chromatograms of selected B vitamins, namely; thiamine, PLP (Pyridoxal 5′-phosphate), pyridoxine, pyridoxal, pyridoxamine, nicotinic acid, pantothenic acid, nicotinamide, CN B12 (cyanocobalamine), MeB12 (methyl-cobalamine), 5MHTF (5-methyl-tetrahydrofolate), FA (folic acid), AdenB12 (adenosyl-cobalamin), FMN (flavin mononucleotide), riboflavin, and biotin, respectively, in accordance with the present disclosure. For FIG. 25A-FIG. 25P, the chromatograms are of standard solution at 3 ppm (μg/ml).

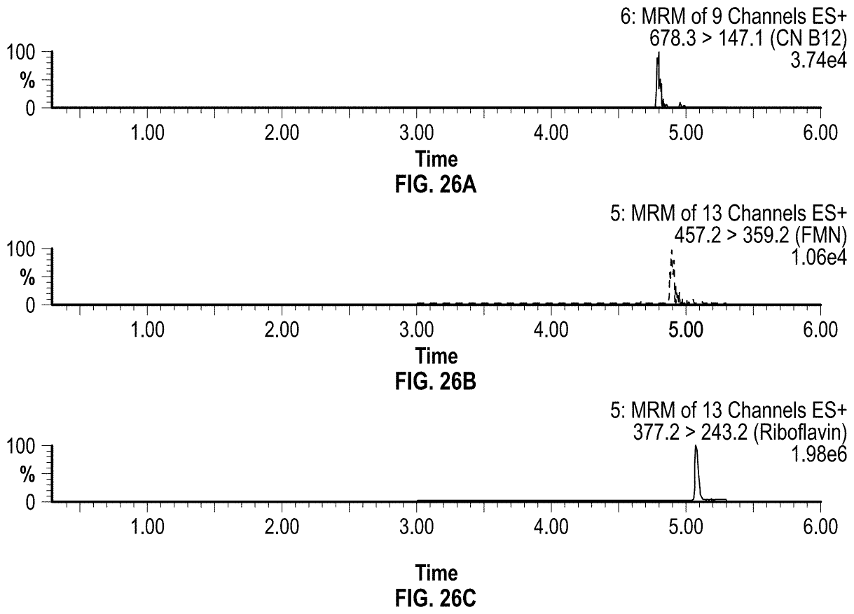

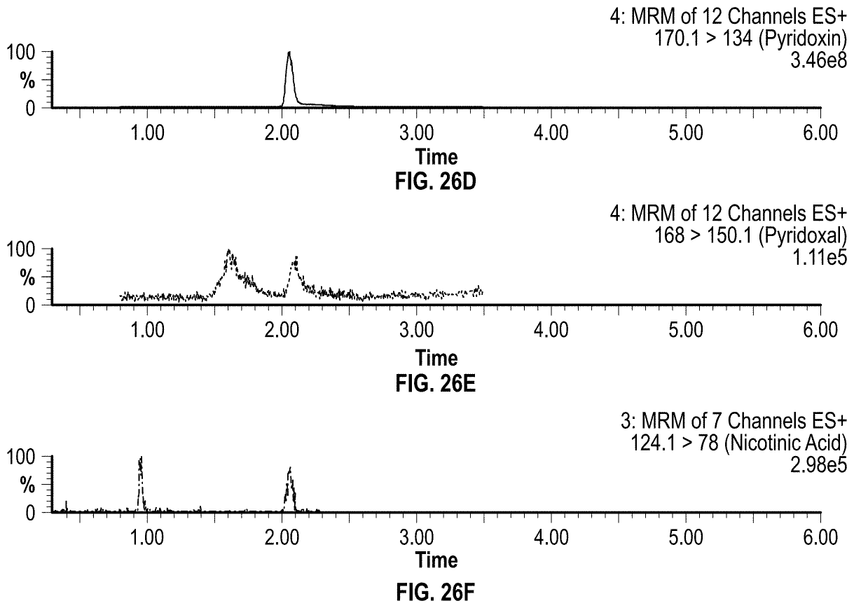

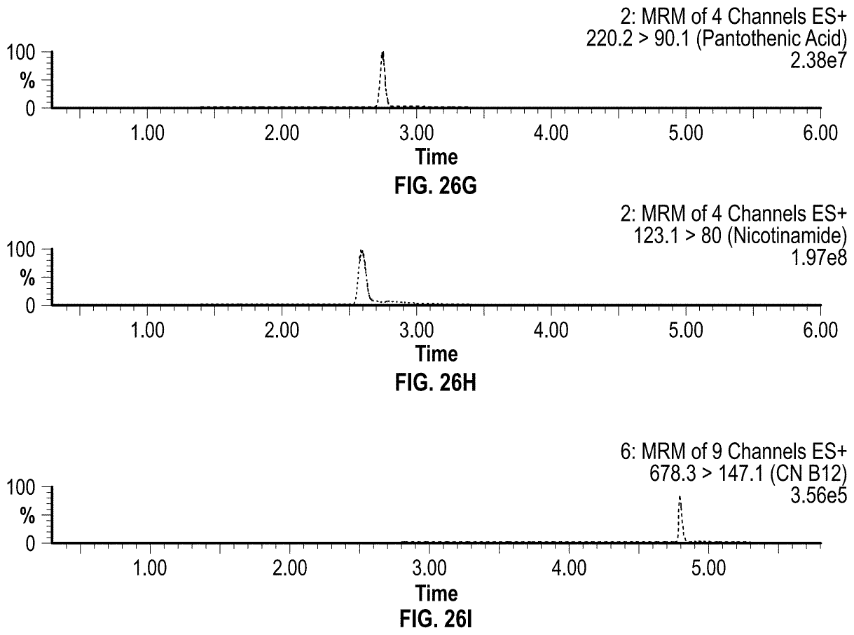

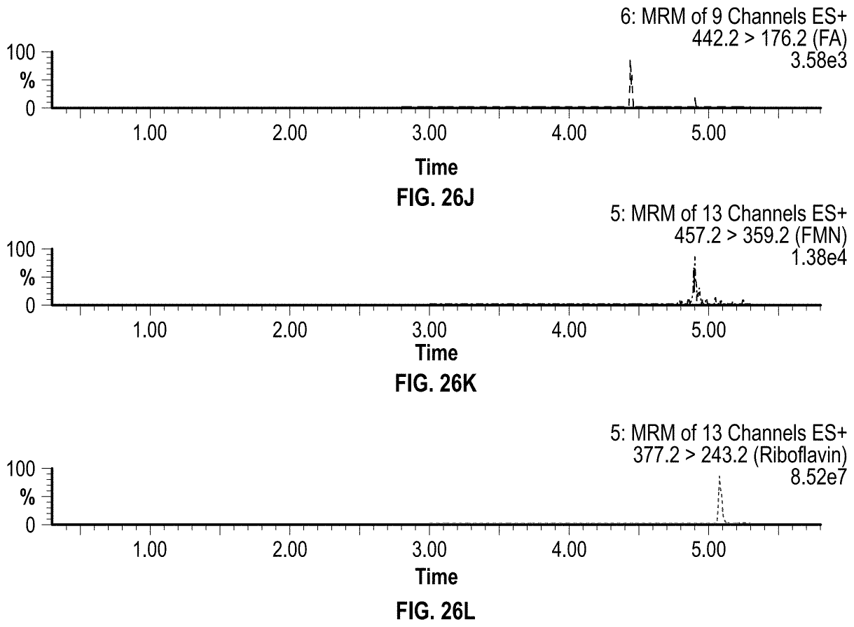

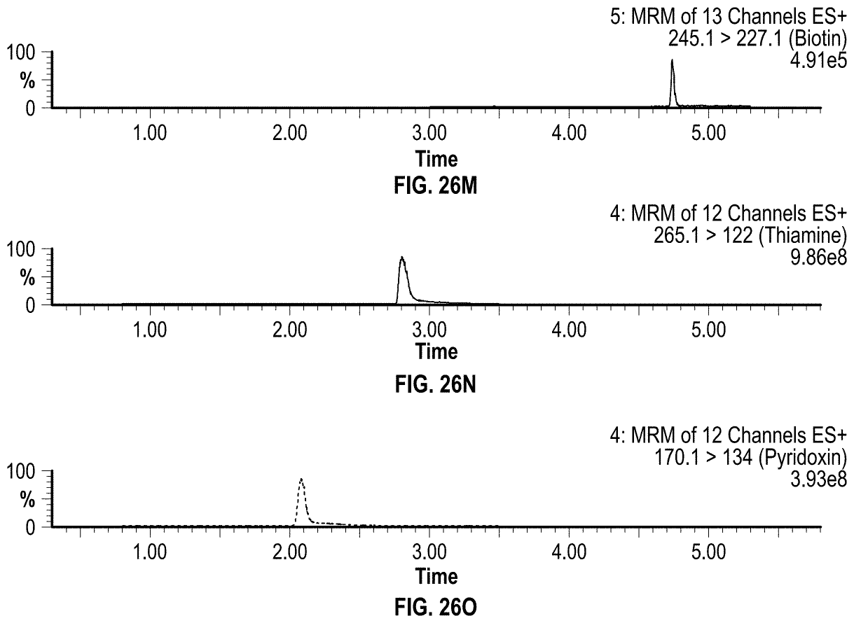

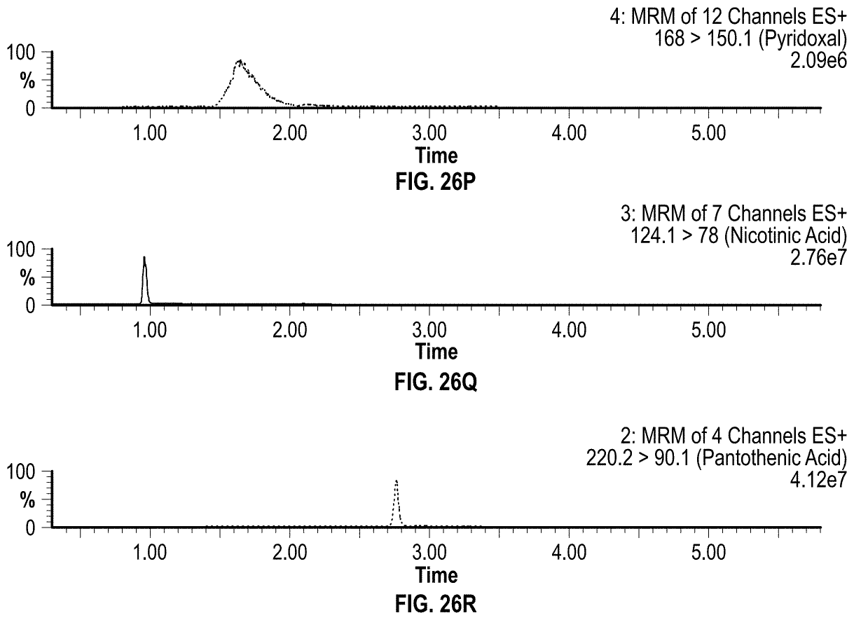

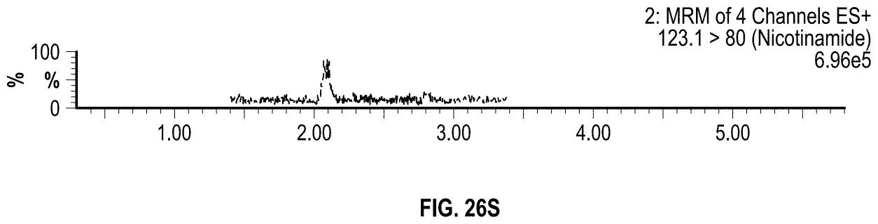

FIG. 26A, FIG. 26B, FIG. 26C, FIG. 26D, FIG. 26E, FIG. 26F, FIG. 26G and FIG. 26H displays a typical chromatogram of selected B vitamins in an energy drink sample. FIG. 26I, FIG. 26J, FIG. 26K, FIG. 26L, FIG. 26M, FIG. 26N, FIG. 26O, FIG. 26P, FIG. 26Q, FIG. 26R and FIG. 26S displays a typical chromatogram of selected B vitamins in a dietary supplement sample. FIG. 26A-FIG. 26H display a typical chromatogram of CN B12, FMN, riboflavin, pyridoxal, nicotinic acid, pantothenic acid, nicotinamide, respectively, in accordance with the present disclosure. FIG. 26I-FIG. 26S display a typical chromatogram of CN B12, FA (folic acid), FMN, riboflavin, biotin, thiamine, pyridoxine, pyridoxal, nicotinic acid, pantothenic acid, and nicotinamide, respectively, in accordance with the present disclosure. As compared to the typical examples of FIG. 25A-FIG. 25P, the present disclosure provides chromatograms with higher response, better peak shapes, and no carry-over. The analysis of B vitamins receives the benefit of higher sensitivity, higher accuracy and precision, and enhanced robustness for the analysis of B vitamins.

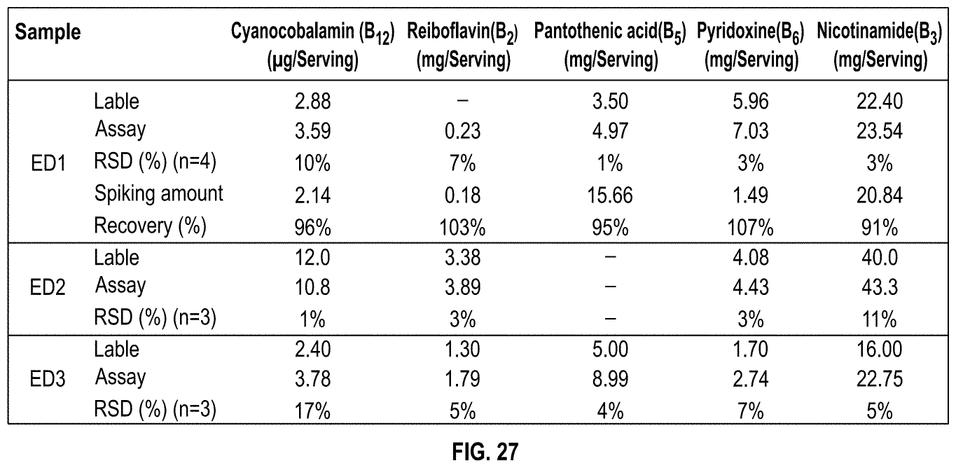

FIG. 27 displays the table of energy drink sample analysis with recovery and repeatability.

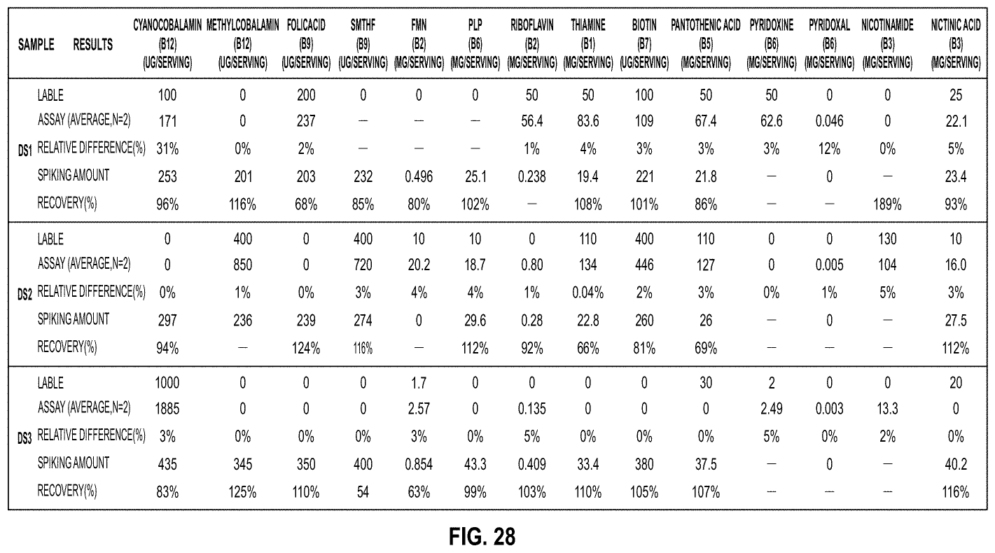

FIG. 28 displays the table of dietary supplement sample analysis with recovery and repeatability. For FIG. 27 and FIG. 28, the results were obtained on a LBS configuration system.

The use of LBS in LC system and column helps to increase the peak area (intensity), reduce peak tailing and peak width for B vitamins, which result in a better detection sensitivity. In addition, the use of LBS in LC system and column helps to reduce the potential carry-over issue for B vitamins.

The present disclosure highlights the benefits of using the LBS configuration for the LC-MS/MS analysis of B-group vitamins. The benefits include sharper and more symmetric peak shapes, greater peak areas, increased peak area (intensity), reduced peak tailing and peak width for B vitamins, which result in a better detection sensitivity. In addition, the use of LBS in LC system and column helps to reduce the potential carry-over issue for B vitamins. These improvements help analysts to quantify B vitamins at lower concentrations, with higher accuracy and precision, and enhanced robustness.

The above aspects and features of the present technology provide numerous advantages over the prior art. In some embodiments, there are numerous benefits incorporating the coating through the column (and in some embodiments through the entire fluidic pathway from sample reservoir to the detector) to define a LBS (e.g., an organosilica coated surface). For example, the present disclosure shows the benefits of reducing secondary interactions, which includes positively impacting chromatographic performance in terms of band broadening, peak tailing, and/or recovery which can then help increase resolution, peak capacity, and/or quantitative accuracy of B vitamins and their vitamers.

1-21. (canceled)

22. A method of separating and analyzing a metal-sensitive sample, the method comprising:
injecting the metal-sensitive sample into a chromatographic system, wherein the chromatographic system comprises a metallic flow path with a low-bind surface coating;
flowing the metal-sensitive sample through the chromatographic system;
separating the metal-sensitive sample, wherein the metal-sensitive sample comprises one or more B vitamin and/or B vitamin vitamer; and
analyzing the separated sample by passing the separated metal-sensitive sample through a mass spectrometer.

23. The method of claim 22, wherein the sample comprises two or more of B vitamins and/or B vitamin vitamers, and wherein analyzing the separated sample comprises simultaneously analyzing the two or more of B vitamins and/or B vitamin vitamers to determine the B vitamins and/or B vitamin vitamers present in the sample.

24. The method of claim 22, wherein prior to injecting the metal-sensitive sample into the chromatographic system, extracting the metal-sensitive sample from a sample matrix or sample matrices.

25. The method of claim 24, wherein extracting the metal-sensitive sample from a sample matrix or sample matrices comprises separate sample preparations for different B vitamins.

26. The method of claim 22, wherein flowing the metal-sensitive sample through the chromatographic system comprises using a single liquid chromatography method to determine the B vitamins and/or B vitamin vitamers.

27. The method of claim 26, wherein the single liquid chromatography method comprises using liquid chromatography-mass spectrometry (LC/MS).

28. The method of claim 27, wherein the liquid chromatography-mass spectrometry (LC/MS) is a tandem quadrupole mass spectrometer (LC-MS/MS).

29. The method of claim 22, wherein the B vitamins or B vitamin vitamers comprise at least one of riboflavin (B₂), flavin mononucleotide (FMN), biotin, nicotinic acid (B₃), nicotinamide (B₃), nicotinamide adenine dinucleotide (NAD), pyridoxine (B₆), pyridoxal (B₆), pyridoxal 5′-phosphate (PLP), thiamine (B₁), thiamine pyrophosphate (TPP), folic acid (B₉), pantothenic acid (B₅), or 5-methyl-tetrahydrofolate (5MTHF).

30. The method of claim 22, wherein the sample is a dietary supplement or an energy drink.

31. A method of separating and analyzing a metal-sensitive sample, the method comprising:
injecting the metal-sensitive sample into a chromatographic system, wherein the chromatographic system comprises a metallic flow path with a coating;
flowing the metal-sensitive sample through the chromatographic system;
separating the metal-sensitive sample, wherein the metal-sensitive sample comprises two or more B vitamins and/or B vitamin vitamers; and
simultaneously analyzing the two or more B vitamins and/or B vitamin vitamers of the separated sample.

32. The method of claim 31, wherein the coating is a low-bind surface coating.

33. The method of claim 31, wherein analyzing the separated sample comprises passing the separated metal-sensitive sample through a mass spectrometer.

34. The method of claim 31, wherein prior to injecting the metal-sensitive sample into the chromatographic system, extracting the metal-sensitive sample from a sample matrix or sample matrices.

35. The method of claim 34, wherein extracting the metal-sensitive sample from a sample matrix or sample matrices comprises separate sample preparations for different B vitamins and/or B vitamin vitamers.

36. The method of claim 31, wherein flowing the metal-sensitive sample through the chromatographic system comprises using a single liquid chromatography method to determine the B vitamins and/or B vitamin vitamers.

37. The method of claim 36, wherein the single liquid chromatography method comprises using liquid chromatography-mass spectrometry (LC/MS).

38. The method of claim 37, wherein the liquid chromatography-mass spectrometry (LC/MS) is a tandem quadrupole mass spectrometer (LC-MS/MS).

39. The method of claim 31, wherein the B vitamins or B vitamin vitamers comprise riboflavin (B₂), flavin mononucleotide (FMN), biotin, nicotinic acid (B₃), nicotinamide (B₃), nicotinamide adenine dinucleotide (NAD), pyridoxine (B₆), pyridoxal (B₆), pyridoxal 5′-phosphate (PLP), thiamine (B₁), thiamine pyrophosphate (TPP), folic acid (B₉), pantothenic acid (B₅), or 5-methyl-tetrahydrofolate (5MTHF).
